SEQUENCE LISTING (126 sequences)

SEQ ID NO: 1 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 2 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSS

SEQ ID NO: 3 - protein, 128 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 4 - protein, 128 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 5 - protein, 126 aa, Artificial Sequence
  Synthetic Construct
EVQLVESGGGLVQAGGSLRLSCAASGRTVSSTAVAWFRQAPGKEREFTAGIGGSVDITYYLDSVKGRFTISKDNTKNTVYLQMNSLKPEDTAVYYCAVRPGRPLITSRDANLYDYWGQGTQVTVSS

SEQ ID NO: 6 - protein, 126 aa, Artificial Sequence
  Synthetic Construct
EVQLLESGGGLVQPGGSLRLSCAASGRTVSSTAVAWFRQAPGKEREFVAGIGGSVDITYYADSVKGRFTISRDNSKNTLYLQMNSLRPEDTAVYYCAVRPGRPLITSRDANLYDYWGQGTLVTVSS

SEQ ID NO: 7 - protein, 126 aa, Artificial Sequence
  Synthetic Construct
EVQLLESGGGLVQPGGSLRLSCAASGRYIDSTAVAWFRQAPGKEREFVAGIGGGVDITYYADSVKGRFTISRDNSKNTLYLQMNSLRPEDTAVYYCAARPGRPLITSRVANLYPYWGQGTLVTVSS

SEQ ID NO: 8 - protein, 126 aa, Artificial Sequence
  Synthetic Construct
EVQLLESGGGLVQPGGSLRLSCAASYRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 9 - protein, 126 aa, Artificial Sequence
  Synthetic Construct
EVQLLESGGGLVQPGGSLRLSCAASGAYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 10 - protein, 126 aa, Artificial Sequence
  Synthetic Construct
EVQLLESGGGLVQPGGSLRLSCAASGRYIDETAVAWFRQAPGKEREFVAGIGGGVDETYYADSVKGRFTISRDNSKNTLYLQMNSLRPEDTAVYYCAARPGRPLITSKVADLYPYWGQGTLVTVSS

SEQ ID NO: 11 - protein, 126 aa, Artificial Sequence
  Synthetic Construct
EVQLLESGGGLVQPGGSLRLSCAASGRYIDETAVAWFRQAPGKEREFVAGIGGGVDQTYYADSVKGRFTISRDNSKNTLYLQMNSLRPEDTAVYYCAARPGRPLITSKVADLYPYWGQGTLVTVSS

SEQ ID NO: 12 - protein, 126 aa, Artificial Sequence
  Synthetic Construct
EVQLLESGGGLVQPGGSLRLSCAASGRYIDETAVAWFRQAPGKEREFVAGIGGGVDITAYADSVKGRFTISRDNSKNTLYLQMNSLRPEDTAVYYCAARPGRPLITSKVADLYPYWGQGTLVTVSS

SEQ ID NO: 13 - protein, 126 aa, Artificial Sequence
  Synthetic Construct
EVQLLESGGGLVQPGGSLRLSCAASGRYIDETAVAWFRQAPGKEREFVAGIGGGVDITEYADSVKGRFTISRDNSKNTLYLQMNSLRPEDTAVYYCAARPGRPLITSKVADLYPYWGQGTLVTVSS

SEQ ID NO: 14 - protein, 126 aa, Artificial Sequence
  Synthetic Construct
EVQLLESGGGLVQPGGSLRLSCAASGRYIDETAVAWFRQAPGKEREFVAGIGGGVDITQYADSVKGRFTISRDNSKNTLYLQMNSLRPEDTAVYYCAARPGRPLITSKVADLYPYWGQGTLVTVSS

SEQ ID NO: 15 - protein, 126 aa, Artificial Sequence
  Synthetic Construct
EVQLLESGGGLVQPGGSLRLSCAASGRYIDETAVAWFRQAPGKEREFVAGIGGGVDITSYADSVKGRFTISRDNSKNTLYLQMNSLRPEDTAVYYCAARPGRPLITSKVADLYPYWGQGTLVTVSS

SEQ ID NO: 16 - protein, 126 aa, Artificial Sequence
  Synthetic Construct
EVQLLESGGGLVQPGGSLRLSCAASGRYIDETAVAWFRQAPGKEREFVAGIGGGVDITTYADSVKGRFTISRDNSKNTLYLQMNSLRPEDTAVYYCAARPGRPLITSKVADLYPYWGQGTLVTVSS

SEQ ID NO: 17 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGKPLITSKVADLYPYWGQGTLVTVSS

SEQ ID NO: 18 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGQPLITSKVADLYPYWGQGTLVTVSS

SEQ ID NO: 19 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGSPLITSKVADLYPYWGQGTLVTVSS

SEQ ID NO: 20 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRELITSKVADLYPYWGQGTLVTVSS

SEQ ID NO: 21 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRQLITSKVADLYPYWGQGTLVTVSS

SEQ ID NO: 22 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRSLITSKVADLYPYWGQGTLVTVSS

SEQ ID NO: 23 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPEITSKVADLYPYWGQGTLVTVSS

SEQ ID NO: 24 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPGITSKVADLYPYWGQGTLVTVSS

SEQ ID NO: 25 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPQITSKVADLYPYWGQGTLVTVSS

SEQ ID NO: 26 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPTITSKVADLYPYWGQGTLVTVSS

SEQ ID NO: 27 - protein, 126 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITEKVADLYPYWGQGTLVTVSS

SEQ ID NO: 28 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCATRPGRPLITSKVADLYPYWGQGTLVTVSS

SEQ ID NO: 29 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARQGRPLITSKVADLYPYWGQGTLVTVSS

SEQ ID NO: 30 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCATRQGRPLITSKVADLYPYWGQGTLVTVSS

SEQ ID NO: 31 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSQVADLYPYWGQGTLVTVSS

SEQ ID NO: 32 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKQADLYPYWGQGTLVTVSS

SEQ ID NO: 33 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVAELYPYWGQGTLVTVSS

SEQ ID NO: 34 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVASLYPYWGQGTLVTVSS

SEQ ID NO: 35 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKQAELYPYWGQGTLVTVSS

SEQ ID NO: 36 - protein, 126 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKQASLYPYWGQGTLVTVSS

SEQ ID NO: 37 - protein, 127 aa, Artificial Sequence
  Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSC

SEQ ID NO: 38 - protein, 5 aa, Artificial Sequence
  Synthetic Construct
  REPEAT (1)..(5)
GGGGQ

SEQ ID NO: 39 - protein, 4 aa, Artificial Sequence
  Synthetic Construct
  REPEAT (1)..(4)
GGGQ

SEQ ID NO: 40 - protein, 5 aa, Artificial Sequence
  Synthetic Construct
  REPEAT (1)..(5)
GGGGS

SEQ ID NO: 41 - protein, 4 aa, Artificial Sequence
  Synthetic Construct
  REPEAT (1)..(4)
PGPQ

SEQ ID NO: 42 - protein, 4 aa, Artificial Sequence
  Synthetic Construct
  REPEAT (1)..(4)
PGPA

SEQ ID NO: 43 - protein, 10 aa, Artificial Sequence
  Synthetic Construct
  REPEAT (5)..(6)
GGGGAPGGGG

SEQ ID NO: 44 - protein, 3 aa, Artificial Sequence
  Synthetic Construct
  REPEAT (1)..(3)
GGE

SEQ ID NO: 45 - protein, 5 aa, Artificial Sequence
  Synthetic Construct
  REPEAT (1)..(5)
GGGGE

SEQ ID NO: 46 - protein, 3 aa, Artificial Sequence
  Synthetic Construct
  REPEAT (1)..(3)
GGK

SEQ ID NO: 47 - protein, 5 aa, Artificial Sequence
  Synthetic Construct
  REPEAT (1)..(5)
GGGGK

SEQ ID NO: 48 - protein, 10 aa, Artificial Sequence
  Synthetic Construct
  REPEAT (5)..(6)
GGGGEPGGGG

SEQ ID NO: 49 - protein, 10 aa, Artificial Sequence
  Synthetic Construct
  REPEAT (5)..(6)
GGGGKPGGGG

SEQ ID NO: 50 - protein, 4 aa, Artificial Sequence
  Synthetic Construct
  REPEAT (1)..(4)
PGPE

SEQ ID NO: 51 - protein, 4 aa, Artificial Sequence
  Synthetic Construct
  REPEAT (1)..(4)
PGPK

SEQ ID NO: 52 - protein, 25 aa, Artificial Sequence
  Synthetic Construct
GGGGQGGGGQGGGGQGGGGQGGGGQ

SEQ ID NO: 53 - protein, 32 aa, Artificial Sequence
  Synthetic Construct
PGPQPGPQPGPQPGPQPGPQPGPQPGPQPGPQ

SEQ ID NO: 54 - protein, 32 aa, Artificial Sequence
  Synthetic Construct
PGPAPGPAPGPAPGPAPGPAPGPAPGPAPGPA

SEQ ID NO: 55 - protein, 24 aa, Artificial Sequence
  Synthetic Construct
GGEGGEGGEGGEGGEGGEGGEGGE

SEQ ID NO: 56 - protein, 25 aa, Artificial Sequence
  Synthetic Construct
GGGGEGGGGEGGGGEGGGGEGGGGE

SEQ ID NO: 57 - protein, 24 aa, Artificial Sequence
  Synthetic Construct
GGKGGKGGKGGKGGKGGKGGKGGK

SEQ ID NO: 58 - protein, 25 aa, Artificial Sequence
  Synthetic Construct
GGGGKGGGGKGGGGKGGGGKGGGGK

SEQ ID NO: 59 - protein, 28 aa, Artificial Sequence
  Synthetic Construct
GGGGAPAPAPAPAPAPAPAPAPAPGGGG

SEQ ID NO: 60 - protein, 28 aa, Artificial Sequence
  Synthetic Construct
GGGGEPEPEPEPEPEPEPEPEPEPGGGG

SEQ ID NO: 61 - protein, 28 aa, Artificial Sequence
  Synthetic Construct
GGGGKPKPKPKPKPKPKPKPKPKPGGGG

SEQ ID NO: 62 - protein, 32 aa, Artificial Sequence
  Synthetic Construct
PGPEPGPEPGPEPGPEPGPEPGPEPGPEPGPE

SEQ ID NO: 63 - protein, 32 aa, Artificial Sequence
  Synthetic Construct
PGPKPGPKPGPKPGPKPGPKPGPKPGPKPGPK

SEQ ID NO: 64 - protein, 10 aa, Artificial Sequence
  Synthetic Construct
GGGSGGSGGG

SEQ ID NO: 65 - protein, 13 aa, Artificial Sequence
  Synthetic Construct
GGGSGGSGGSGGG

SEQ ID NO: 66 - protein, 20 aa, Mus musculus
METDTLLLWVLLLWVPGSTG

SEQ ID NO: 67 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 68 - protein, 42 aa, Homo sapiens
YAEGTFISDYSIAMDKIHQQDFVNWLLAQKGKKNDWKHNITQ

SEQ ID NO: 69 - protein, 445 aa, Homo sapiens
RAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 70 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 71 - protein, 37 aa, Homo sapiens
HDEFERHAEGTFTSDVSSYLEGQAAKEFIAWLVKGRG

SEQ ID NO: 72 - protein, 31 aa, Homo sapiens
HAEGTFTSDVSSYLEGQAAKEFIAWLVKGRG

SEQ ID NO: 73 - protein, 30 aa, Homo sapiens
HAEGTFTSDVSSYLEGQAAKEFIAWLVKGR

SEQ ID NO: 74 - protein, 440 aa, Homo sapiens
RPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 75 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 76 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 77 - protein, 394 aa, Homo sapiens
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 78 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 79 - protein, 21 aa, Homo sapiens
GIVEQCCTSICSLYQLENYCN

SEQ ID NO: 80 - protein, 30 aa, Homo sapiens
FVNQHLCGSHLVEALYLVCGERGFFYTPKT

SEQ ID NO: 81 - protein, 731 aa, Homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPS

SEQ ID NO: 82 - protein, 620 aa, Homo sapiens
SLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 83 - protein, 133 aa, Homo sapiens [sequence omitted]

SEQ ID NO: 84 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 85 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 86 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 87 - protein, 640 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 88 - protein, 222 aa, Homo sapiens
SGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQK

SEQ ID NO: 89 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 90 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 91 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 92 - protein, 24 aa, Homo sapiens
QLYSALANKCCHVGCTKRSLARFC

SEQ ID NO: 93 - protein, 29 aa, Homo sapiens
DSWMEEVIKLCGRELVRAQIAICGMSTWS

SEQ ID NO: 94 - protein, 757 aa, Homo sapiens
MTSGSVFFYILIFGKYFSHGGGQDVKCSLGYFPCGNITKCLPQLLHCNGVDDCGNQADEDNCGDNNGWSLQFDKYFASYYKMTSQYPFEAETPECLVGSVPVQCLCQGLELDCDETNLRAVPSVSSNVTAMSLQWNLIRKLPPDCFKNYHDLQKLYLQNNKITSISIYAFRGLNSLTKLYLSHNRITFLKPGVFEDLHRLEWLIIEDNHLSRISPPTFYGLNSLILLVLMNNVLTRLPDKPLCQHMPRLHWLDLEGNHIHNLRNLTFISCSNLTVLVMRKNKINHLNENTFAPLQKLDELDLGSNKIENLPPLIFKDLKELSQLNLSYNPIQKIQANQFDYLVKLKSLSLEGIEISNIQQRMFRPLMNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 95 - protein, 39 aa, Homo sapiens
SYSMEHFRWGKPVGKKRRPVKVYPNGAEDESAEAFPLEF

SEQ ID NO: 96 - protein, 297 aa, Homo sapiens
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 97 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 98 - protein, 320 aa, Homo sapiens
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGS

SEQ ID NO: 99 - protein, 274 aa, Artificial Sequence
  Synthetic Construct
HGEGTFTSDVSSYLEEQAAKEFIAWLVKGRGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 338 aa, Artificial Sequence
  Synthetic Construct
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 101 - protein, 336 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQMAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 102 - protein, 222 aa, Artificial Sequence
  Synthetic Construct
SHLVKCAEKEKTFCVNGGECFMVKDLSNPSRYLCKCPNEFTGDRCQNYVMASFYKHLGIEFMEAEELYQGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 103 - protein, 220 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQSHLVKCAEKEKTFCVNGGECFMVKDLSNPSRYLCKCPNEFTGDRCQNYVMASFYKHLGIEFMEAEELYQ

SEQ ID NO: 104 - protein, 260 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 105 - protein, 286 aa, Artificial Sequence
  Synthetic Construct
APQSSSTQQTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFAQSIISTLTGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 106 - protein, 284 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQAPQSSSTQQTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 107 - protein, 184 aa, Artificial Sequence
  Synthetic Construct
HGEGTFTSDVSSYLEEQAAKEFIAWLVKGGGGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 108 - protein, 382 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 109 - protein, 214 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 225 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 111 - protein, 367 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 112 - protein, 376 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 113 - protein, 214 aa, Artificial Sequence; identical to SEQ ID NO: 109
  Synthetic Construct

SEQ ID NO: 114 - protein, 225 aa, Artificial Sequence; identical to SEQ ID NO: 110
  Synthetic Construct

SEQ ID NO: 115 - protein, 365 aa, Artificial Sequence
  Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 183 aa, Artificial Sequence
  Synthetic Construct
HGEGTFTSDVSSYLEEQAAKEFIAWLVKGGGGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSC

SEQ ID NO: 117 - protein, 183 aa, Artificial Sequence; identical to SEQ ID NO: 116
  Synthetic Construct
  MOD_RES (183)..(183): Cys at position 183 is chemically modified on the sulfur group of the side chain, forming a thioether linkage with maleamide-(PEG)12-(ACTH in a C-termina to N-terminal orientation)

SEQ ID NO: 118 - protein, 127 aa, Artificial Sequence; identical to SEQ ID NO: 37
  Synthetic Construct
  MOD_RES (127)..(127): Cys at position 127 is chemically modified on the sulfur group of the side chain, forming a thioether linkage with maleamide-(PEG)12-(ACTH in a C-terminal to N-terminal orientation)

SEQ ID NO: 119 - protein, 25 aa, Artificial Sequence
  Synthetic Construct
  MOD_RES (25)..(25): Lys at position 25 is chemically modified by conjugation of the epsilon-amino group of the side chain with (PEG)12-maleamide
  MOD_RES (25)..(25): AMIDATION
SYSMEHFRWGKPVGKKRRPVKVYPK

SEQ ID NO: 120 - protein, 225 aa, Artificial Sequence; identical to SEQ ID NO: 110
  Synthetic Construct

SEQ ID NO: 121 - protein, 214 aa, Artificial Sequence; identical to SEQ ID NO: 109
  Synthetic Construct

SEQ ID NO: 122 - protein, 216 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 123 - protein, 136 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAE

SEQ ID NO: 124 - protein, 127 aa, Artificial Sequence
  Synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSC

SEQ ID NO: 125 - protein, 128 aa, Artificial Sequence
  Synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 126 - protein, 128 aa, Artificial Sequence
  Synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGTRPGRPLITSKVADLYPYWGQGTLVTVSSPP

(19) United States
(12) Patent Application Publication
(10) Pub. No.: US 2024/0316154 A1
(43) Pub. Date: Sep. 26, 2024

(54) HALF-LIFE EXTENDING MOIETIES AND METHODS OF USING THE SAME

(71) Applicant: Eli Lilly and Company, Indianapolis, IN (US)

(72) Inventors: Andrea Ferrante, La Mesa, CA (US); Josef George Heuer, Carmel, IN (US); Stacey Lynn Lee, San Diego, CA (US); Petra Verdino, San Diego, CA (US)

(21) Appl. No.: 18/259,500

(22) PCT Filed: Feb. 1, 2022
(86) PCT No.: PCT/US2022/014728

§ 371 Date: Jun. 27, 2023

Related U.S. Application Data
(60) Provisional application No. 63/144,696, filed on Feb. 2, 2021.

(51) Int. Cl. A61K 38/26; A61K 38/18; A61K 38/20; A61K 38/22; A61K 45/06; C07K 14/00

(52) U.S. Cl. CPC A61K 38/26; A61K 38/1883; A61K 38/2013; A61K 38/2221; A61K 45/06; C07K 14/00

(57) ABSTRACT

The disclosure relates generally to biology and medicine, and more particularly it relates to compounds acting as half-life (t½)-extending moieties for use with therapeutics, especially for improving t½ of biological-based therapeutics (i.e., biotherapeutics or biologics). The disclosure further relates to fusions and conjugates that include one or more of the compounds acting as t½-extending moieties, as well as pharmaceutical compositions including the same and their use in treating various conditions, diseases or disorders.

The disclosure relates generally to biology and medicine, and more particularly it relates to single domain antibodies known as variable domains of heavy chain-only antibodies (VHH) that are engineered/modified to act as half-life (t½)-extending moieties for use with therapeutic agents, especially for improving t½ of biological-based therapeutic agents (i.e., biotherapeutics or biologics). The disclosure further relates to fusions and conjugates that include one or more of the VHH-based t½-extending moieties and a therapeutic agent, as well as pharmaceutical compositions including the same and their use in treating various conditions, diseases or disorders.

Biotherapeutics are native or modified components of physiological pathways and tend to be highly selective, efficacious and safe. However, they come with some limitations. One limitation, and with few exceptions, is that biotherapeutics cannot be orally administered. Another limitation is that many biotherapeutics have a relatively short t½ when used in a clinical setting.

Several strategies exist for extending the t½ of biotherapeutics, which can improve their pharmacokinetic (PK) and/or pharmacodynamic (PD) profiles. Such strategies typically use bulking moieties or neonatal Fc receptor (FcRn)-mediated recycling. In this manner, antibodies (Abs) or fragments thereof (e.g., Fab, Fc, etc.); polymers such as polyethylene glycol (PEG), polysialic acid (PSA), hyaluronic acid (HA) and hydroxy-ethyl-starch (HES); fatty acids and other lipids; N- or O-glycosylation; and serum albumin or other plasma proteins (such as transferrin), can be covalently and/or non-covalently bound to a given biotherapeutic to extend its t½. See, e.g., Hamers-Casterman et al. (1993) Nature 363:446-448; Harmsen & Haard (2007) Appl. Microbiol. Biotechnol. 77:13-22; Kontermann (2016) Expert Opin. Biol. Ther. 16:903-915; Müller et al. (2012) MAbs 4:673-685; Podust et al. (2013) Protein Eng. Des. Sel. 26:743-753; Strohl (2015) BioDrugs 29:215-239; and Werle U Bernkop-Schnurch (2006) Amino Acids 30:351-367.

Despite the vast number of t½-extending strategies, there is a need for additional structures for extending or improving PK properties, such as the t½, of biotherapeutics.

To address this need, the disclosure first describes compounds that can be used as t½-extending moieties for biotherapeutics. In one instance, a compound is provided that includes an amino acid sequence of selected from any one of SEQ ID NOS: 1 to 37 and 124 to 126.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:1). Alternatively, the compound can have an amino acid sequence having at least about 90% to about 99% sequence similarity to SEQ ID NO:1.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSS (SEQ ID NO:2). Alternatively, the compound can have an amino acid sequence having at least about 90% to about 99% sequence similarity to SEQ ID NO:2.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSPP (SEQ ID NO:3). Alternatively, the compound can have an amino acid sequence having at least about 90% to about 99% sequence similarity to SEQ ID NO:3.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSPP (SEQ ID NO:4). Alternatively, the compound can have an amino acid sequence having at least about 90% to about 99% sequence similarity to SEQ ID NO:4.

In another instance, a compound is provided that includes an amino acid sequence of: EVOLVESGGGLVQAGGSLRLSCAASGRTVSSTAVAWFRQAPGKEREFTAGIGGS VDITYYLDSVKGRFTISKDNTKNTVYLQMNSLKPEDTAVYYCAVRPGRPLITSRD ANLYDYWGQGTQVTVSS (SEQ ID NO:5). Alternatively, the compound can have an amino acid sequence having at least about 90% to about 99% sequence similarity to SEQ ID NO:5.

In another instance, a compound is provided that includes an amino acid sequence of: EVQLLESGGGLVQPGGSLRLSCAASGRTVSSTAVAWFRQAPGKEREFV AGIGGSVDITYYADSVKGRFTISRDNSKNTLYLQMNSLRPEDTAVYYCAVRPGRP LITSRDANLYDYWGQGTLVTVSS (SEQ ID NO:6). Alternatively, the compound can have an amino acid sequence having at least about 90% to about 99% sequence similarity to SEQ ID NO:6.

In another instance, a compound is provided that includes an amino acid sequence of: EVQLLESGGGLVQPGGSLRLSCAASGRYIDSTAVAWFRQAPGKEREFVAGIGGG VDITYYADSVKGRFTISRDNSKNTLYLQMNSLRPEDTAVYYCAARPGRPLITSRV ANLYPYWGQGTLVTVSS (SEQ ID NO:7). Alternatively, the compound can have an amino acid sequence having at least about 90% to about 99% sequence similarity to SEQ ID NO:7.

In another instance, a compound is provided that includes an amino acid sequence of: EVQLLESGGGLVQPGGSLRLSCAASYRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:8). Alternatively, the compound can have an amino acid sequence having at least about 90% to about 99% sequence similarity to SEQ ID NO:8.

In another instance, a compound is provided that includes an amino acid sequence of: EVQLLESGGGLVQPGGSLRLSCAASGAYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:9). Alternatively, the compound can have an amino acid sequence having at least about 90% to about 99% sequence similarity to SEQ ID NO:9.

In another instance, a compound is provided that includes an amino acid sequence of: EVQLLESGGGLVQPGGSLRLSCAASGRYIDETAVAWFRQAPGKEREFVAGIGGG VDETYYADSVKGRFTISRDNSKNTLYLQMNSLRPEDTAVYYCAARPGRPLITSKV ADLYPYWGQGTLVTVSS (SEQ ID NO:10). Alternatively, the compound can have an amino acid sequence having at least about 90% to about 99% sequence similarity to SEQ ID NO:10.

In another instance, a compound is provided that includes an amino acid sequence of: EVQLLESGGGLVQPGGSLRLSCAASGRYIDETAVAWFRQAPGKEREFVAGIGGG VDQTYYADSVKGRFTISRDNSKNTLYLQMNSLRPEDTAVYYCAARPGRPLITSK VADLYPYWGQGTLVTVSS (SEQ ID NO:11). Alternatively, the compound can have an amino acid sequence having at least about 90% to about 99% sequence similarity to SEQ ID NO:11.

In another instance, a compound is provided that includes an amino acid sequence of: EVQLLESGGGLVQPGGSLRLSCAASGRYIDETAVAWFRQAPGKEREFVAGIGGG VDITAYADSVKGRFTISRDNSKNTLYLQMNSLRPEDTAVYYCAARPGRPLITSKV ADLYPYWGQGTLVTVSS (SEQ ID NO:12). Alternatively, the compound can have an amino acid sequence having at least about 90% to about 99% sequence similarity to SEQ ID NO:12.

In another instance, a compound is provided that includes an amino acid sequence of: EVQLLESGGGLVQPGGSLRLSCAASGRYIDETAVAWFRQAPGKEREFVAGIGGG VDITEYADSVKGRFTISRDNSKNTLYLQMNSLRPEDTAVYYCAARPGRPLITSKV ADLYPYWGQGTLVTVSS (SEQ ID NO:13). Alternatively, the compound can have an amino acid sequence having at least about 90% to about 99% sequence similarity to SEQ ID NO:13.

In another instance, a compound is provided that includes an amino acid sequence of: EVQLLESGGGLVQPGGSLRLSCAASGRYIDETAVAWFRQAPGKEREFVAGIGGG VDITQYADSVKGRFTISRDNSKNTLYLQMNSLRPEDTAVYYCAARPGRPLITSKV ADLYPYWGQGTLVTVSS (SEQ ID NO:14). Alternatively, the compound can have an amino acid sequence having at least about 90% to about 99% sequence similarity to SEQ ID NO: 14.

In another instance, a compound is provided that includes an amino acid sequence of: EVQLLESGGGLVQPGGSLRLSCAASGRYIDETAVAWFRQAPGKEREFVAGIGGG VDITSYADSVKGRFTISRDNSKNTLYLQMNSLRPEDTAVYYCAARPGRPLITSKV ADLYPYWGQGTLVTVSS (SEQ ID NO:15). Alternatively, the compound can have an amino acid sequence having at least about 90% to about 99% sequence similarity to SEQ ID NO:15.

In another instance, a compound is provided that includes an amino acid sequence of: EVQLLESGGGLVQPGGSLRLSCAASGRYIDETAVAWFRQAPGKEREFVAGIGGG VDITTYADSVKGRFTISRDNSKNTLYLQMNSLRPEDTAVYYCAARPGRPLITSKV ADLYPYWGQGTLVTVSS (SEQ ID NO:16). Alternatively, the compound can have an amino acid sequence having at least about 90% to about 99% sequence similarity to SEQ ID NO:16.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGKPLITSKV ADLYPYWGQGTLVTVSS (SEQ ID NO:17). Alternatively, the compound can have an amino acid sequence having at least about 90% to about 99% sequence similarity to SEQ ID NO:17.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGQPLITSKV ADLYPYWGQGTLVTVSS (SEQ ID NO:18). Alternatively, the compound can have an amino acid sequence having at least about 90% to about 99% sequence similarity to SEQ ID NO:18.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGSPLITSKV ADLYPYWGQGTLVTVSS (SEQ ID NO:19). Alternatively, the compound can have an amino acid sequence having at least about 90% to about 99% sequence similarity to SEQ ID NO:19.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRELITSKV ADLYPYWGQGTLVTVSS (SEQ ID NO:20). Alternatively, the compound can have an amino acid sequence having at least about 90% to about 99% sequence similarity to SEQ ID NO:20.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRQLITSKV ADLYPYWGQGTLVTVSS (SEQ ID NO:21). Alternatively, the compound can have an amino acid sequence having at least about 90% to about 99% sequence similarity to SEQ ID NO:21.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRSLITSKV ADLYPYWGQGTLVTVSS (SEQ ID NO:22). Alternatively, the compound can have an amino acid sequence having at least about 90% to about 99% sequence similarity to SEQ ID NO:22.

In another instance, a compound is provided that includes an amino acid sequence of:

EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPEITSKV ADLYPYWGQGTLVTVSS (SEQ ID NO:23). Alternatively, the compound can have an amino acid sequence having at least about 90% to about 99% sequence similarity to SEQ ID NO:23.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPGITSKV ADLYPYWGQGTLVTVSS (SEQ ID NO:24). Alternatively, the compound can have an amino acid sequence having at least about 90% to about 99% sequence similarity to SEQ ID NO:24.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPQITSKV ADLYPYWGQGTLVTVSS (SEQ ID NO:25). Alternatively, the compound can have an amino acid sequence having at least about 90% to about 99% sequence similarity to SEQ ID NO:25.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPTITSKV ADLYPYWGQGTLVTVSS (SEQ ID NO:26). Alternatively, the compound can have an amino acid sequence having at least about 90% to about 99% sequence similarity to SEQ ID NO:26.

In another instance, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITEKV ADLYPYWGQGTLVTVSS (SEQ ID NO:27). Alternatively, the compound can have an amino acid sequence having at least about 90% to about 99% sequence similarity to SEQ ID NO:27.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCATRPGRPLITSKV ADLYPYWGQGTLVTVSS (SEQ ID NO:28). Alternatively, the compound can have an amino acid sequence having at least about 90% to about 99% sequence similarity to SEQ ID NO:28.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARQGRPLITSKV ADLYPYWGQGTLVTVSS (SEQ ID NO:29). Alternatively, the compound can have an amino acid sequence having at least about 90% to about 99% sequence similarity to SEQ ID NO:29.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCATRQGRPLITSKV ADLYPYWGQGTLVTVSS (SEQ ID NO:30). Alternatively, the compound can have an amino acid sequence having at least about 90% to about 99% sequence similarity to SEQ ID NO:30.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSQV ADLYPYWGQGTLVTVSS (SEQ ID NO:31). Alternatively, the compound can have an amino acid sequence having at least about 90% to about 99% sequence similarity to SEQ ID NO:31.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKQ ADLYPYWGQGTLVTVSS (SEQ ID NO:32). Alternatively, the compound can have an amino acid sequence having at least about 90% to about 99% sequence similarity to SEQ ID NO:32.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV AELYPYWGQGTLVTVSS (SEQ ID NO:33). Alternatively, the compound can have an amino acid sequence having at least about 90% to about 99% sequence similarity to SEQ ID NO:33.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ASLYPYWGQGTLVTVSS (SEQ ID NO:34). Alternatively, the compound can have an amino acid sequence having at least about 90% to about 99% sequence similarity to SEQ ID NO:34.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKQ AELYPYWGQGTLVTVSS (SEQ ID NO:35). Alternatively, the compound can have an amino acid sequence having at least about 90% to about 99% sequence similarity to SEQ ID NO:35.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKQ ASLYPYWGQGTLVTVSS (SEQ ID NO:36). Alternatively, the compound can have an amino acid sequence having at least about 90% to about 99% sequence similarity to SEQ ID NO:36.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSC (SEQ ID NO:37). Alternatively, the compound can have an amino acid sequence having at least about 90% to about 99% sequence similarity to SEQ ID NO:37.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCGARPGRPLITSKV ADLYPYWGQGTLVTVSSC (SEQ ID NO:124). Alternatively, the compound can have an amino acid sequence having at least about 90% to about 99% sequence similarity to SEQ ID NO: 124.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCGARPGRPLITSKV ADLYPYWGQGTLVTVSSPP (SEQ ID NO:125). Alternatively, the compound can have an amino acid sequence having at least about 90% to about 99% sequence similarity to SEQ ID NO:125.

In another instance, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCGTRPGRPLITSKV ADLYPYWGQGTLVTVSSPP (SEQ ID NO:126). Alternatively, the compound can have an amino acid sequence having at least about 90% to about 99% sequence similarity to SEQ ID NO:126.

Second, the disclosure describes compounds that include at least one of a VHH-based t½-extending moiety as described herein and a biotherapeutic. In some instances, the compounds can include a structure from an amino-terminus (N-terminus) to a carboxy-terminus (C-terminus) of:
M-X₁,
X₁-M,
M-X₂,
X₂-M,
M-L₁-X₁,
M-L₂-X₁,
M-L₁-X₂,
M-L₂-X₂,
X₁-L₁-M,
X₁-L₂-M,
X₂-L₁-M,
X₂-L₂-M,
X₁-M-X₂,
X₂-M-X₁,
X₁-L₁-M-X₂,
X₂-L₁-M-X₁,
X₁-M-L₁-X₂,
X₂-M-L₁-X₁,
X₁-L₁-M-L₂-X₂,
X₂-L₁-M-L₂-X₁,
M-L₁-X₁-L₂-X₂,
M-L₁-X₂-L₂-X₁,
X₁-L₂-X₂-L₁-M,
X₂-L₂-X₁-L₁-M, or
M-L₁-X₁ combined with M-L₂-X₂ (i.e., non-covalently associated),
where M is a compound acting as a t½-extending moiety and having an amino acid sequence selected from SEQ ID NOS: 1 to 37 and 124 to 126 or having an amino acid sequence having at least about 90% to about 99% sequence similarity thereto, where L₁ (if present) is a first linker, where L₂ (if present) is a second linker, where X₁ is a biotherapeutic protein, peptide or oligomer, and where X₂ is a biotherapeutic protein, peptide or oligomer that can be the same as or distinct from X₁ (such as when the biotherapeutic is a homodimer, or when the biotherapeutic is a heterodimer X₁ can be one chain (a-chain) and X₂ can be another chain (β-chain) thereof). X₁ and X₂ can also be fully distinct from one another. In some instances, L₁ can have an amino acid sequence of (GGGGQ)ₙ (SEQ ID NO:38), (GGGQ)ₙ (SEQ ID NO:39), (GGGGS)ₙ (SEQ ID NO:40), (PGPQ)ₙ (SEQ ID NO:41), (PGPA)ₙ (SEQ ID NO:42), GGGG(AP)ₙGGGG (SEQ ID NO:43), (GGE)ₙ (SEQ ID NO:44), (GGGGE)ₙ (SEQ ID NO:45), (GGK)ₙ (SEQ ID NO:46), (GGGGK)ₙ (SEQ ID NO:47), GGGG(EP)ₙGGGG (SEQ ID NO:48), GGGG(KP)ₙGGGG, (SEQ ID NO:49), (PGPE)ₙ (SEQ ID NO:50) or (PGPK)ₙ (SEQ ID NO:51), where n can be from 1 to 15, especially from about 5 to about 10. In other instances, L₁ can have an amino acid sequence selected from SEQ ID NOS:52 to 63. In still other instances, L₁ can have one or more additions, deletions, insertions or substitutions such that L₁ has an amino acid sequence having at least about 90% to about 99% sequence similarity to any one of SEQ ID NOS:52 to 63.

In some instances, L₂ can have an amino acid sequence selected from SEQ ID NO:64 to 65. In other instances, L₂ can have one or more additions, deletions, insertions or substitutions such that L₂ has an amino acid sequence having at least about 90% to about 99% sequence similarity to any one of SEQ ID NOS:64 to 65.

In still other instances, L₁ or L₂ can be a polymer such as a polyethylene glycol (PEG), especially (PEG)ₙ, where n can be from 1 to 20.

In some instances, X₁, X₂ or X₁/X₂ is a peptide or protein (and even an oligomer, e.g., a homodimer or heterodimer that may or may not be covalently linked). Examples of such peptides or proteins include, but are not limited to, an antibody (Ab), an antibody fragment (e.g., Fab, scFv, Fab-Fab, VH, VL or VHH of different specificity), a ciliary neurotrophic factor (CNTF), growth/differentiation factor 15 (GDF15), an incretin (INC), an interleukin (IL), a neuregulin (NRG), or a hormone. In certain instances, the INC can be insulin (INS), glucose-dependent insulinotropic peptide (GIP), glucagon-like peptide-1 (GLP-1), GIP/GLP-1, or even an INC having triple receptor activity (i.e., glucagon-GIP-GLP-1 receptor activity). In certain instances, the IL is interleukin-2 (IL-2). In certain instances, the NRG is neuregulin 1 (NRG1) or neuregulin 4 (NRG4). In certain instances, the hormone is adrenocorticotropic hormone (ACTH) or relaxin-2 (RLN-2). In certain instances, the Fab binds to GITR; and in some instances the Fab binds to GITR and is a GITR antagonist.

In particular instances, the compounds can have an amino acid sequence of any one of SEQ ID NO: 100 to 118. Alternatively, the compounds can have at least about 90% to about 99% sequence similarity to an amino acid sequence of any one of SEQ ID NOS: 100 to 118.

Third, the disclosure describes pharmaceutical compositions that include at least one compound herein and a pharmaceutically acceptable carrier.

Fourth, the disclosure describes methods of using the compounds and pharmaceutical compositions for medicaments and for extending the t½ of biotherapeutics.

Fifth, the disclosure describes uses of the compounds herein in the manufacture of medicaments and in extending the t½ of biotherapeutics.

An advantage of the t½-extending moieties and compounds including the same is that they can be chemically or recombinantly synthesized as a single-chain polypeptide (i.e., monomeric) and thus do not require endoproteolytic processing for biological activity. It is contemplated, however, that in some instances, the compounds acting as t½-extending moieties can be fused not only to single-chain peptides and proteins but also to peptides with more than one chain, for example, two-chain peptides, multi-chain peptides and proteins as well. On the compounds acting as t½-extending moieties, one can chemically conjugate not only to the N- and C-terminus but also to any surface-exposed amino acid of the t½-extending moieties (with the proviso that such conjugation does not entirely abrogate albumin binding).

An advantage of the compounds acting as t½-extending moieties and compounds including the same is that the t½-extending moieties provide an extended duration of action in mammals such as humans and can have a t½ of about 20 days to about 30 days, thereby allowing for at least weekly or biweekly administration when compared to native peptides and proteins, which can improve compliance and can improve quality of life, especially in cases of chronic diseases requiring life-long therapy.

An advantage of the compounds acting as t½-extending moieties herein is that they have tunable pharmacokinetics achieved by changing albumin affinity of the t½-extending moieties.

An advantage of the compounds acting as t½-extending moieties herein is that they may enable recombinant expression in standard manufacturing organisms such as yeast, mammalian or prokaryotes.

Moreover, and an advantage of the compounds acting as t½-extending moieties herein is that they have similar binding not only to human serum albumin but also to monkey, mouse, rat, dog and pig serum albumin, which allows for pharmacodynamic, pharmacokinetic and toxicology studies to more readily translate from these species to humans. As such, the t½-extending moieties herein can be used not only for treating humans but also for treating animals.

### DETAILED DESCRIPTION

Unless defined otherwise, all technical and scientific terms used herein have the same meaning as commonly understood by one of skill in the art to which the disclosure pertains. Although any methods and materials similar to or equivalent to those described herein can be used in the practice or testing of the analogs, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

### Definitions

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of receptors, “activity,” “activate,” “activating” and the like means a capacity of a compound, such as a fusion herein, to bind to and induce a response at the receptor(s), as measured using assays known in the art, such as the in vitro assays described below.

As used herein, “adrenocorticotropic hormone” or “ACTH” means an ACTH obtained or derived from any species, such as a mammalian species, especially a human. ACTH includes both native ACTH (i.e., full-length) and variations thereof (i.e., additions, deletions, insertions and/or substitutions of native ACTH). One sequence for ACTH is set forth in SEQ ID NO:95 (UniProt/SwissProt Database Accession No. P01189). In humans, ACTH binds to the ACTH receptor (ACTHR, also known as the melanocortin receptor 2 or MC2R), and one sequence for ACTHR is set forth in SEQ ID NO:96 (UniProt/SwissProt Database Accession No. Q01718). ACTHR is a G protein-coupled receptor located on the external cell plasma membrane and is coupled to Gas and upregulates levels of cAMP by activating adenylyl cyclase.

As used herein, “amino acid” means a molecule that, from a chemical standpoint, is characterized by a presence of one or more amine groups and one or more carboxylic acid groups, and may contain other functional groups. As is known in the art, there is a set of twenty amino acids that are designated as standard amino acids and that can be used as building blocks for most of the peptides/proteins produced by any living being. The amino acid sequences in the disclosure contain the standard single letter or three letter codes for the twenty naturally occurring amino acids.

As used herein, “analog” means a compound, such as a synthetic peptide or polypeptide, that activates a target receptor and that elicits at least one in vivo or in vitro effect elicited by a native agonist for that receptor.

As used herein, “biotherapeutic” and the like means an amino acid- or nucleic acid-based compounds such as antibodies, coagulation factors, clotting factors, cytokines, enzymes, growth factors, hormones, and fragments thereof, having at least one therapeutic activity/applicability, as well as therapeutic DNA and/or RNA molecules.

As used herein, “CNTF” or “ciliary neutrotrophic factor” means a CNTF obtained or derived from any species, such as a mammalian species, especially a human. CNTF includes both native CNTF (i.e., full-length) and variations thereof (i.e., additions, deletions, insertions and/or substitutions of native CNTF). One sequence for CNTF is set forth in SEQ ID NO:97 (UniProt/SwissProt Database Accession No. P26441). In humans, CNTF binds to the CNTFα-receptor (CNTFRα), and one sequence for CNTFRα is set forth in SEQ ID NO:98 (UniProt/SwissProt Database Accession No. P26992). CNTFRα also uses two signal-transducing transmembrane subunits, LIFRβ and gp130, which together activate the Jak-STAT signaling pathway. See, Stahl et al. (1994) Science 263:92-95 and Stahl & Yancopoulos (1994) J. Neurobiol. 25:1454-1466.

As used herein, “conservative substitution” means a variant of a reference peptide or polypeptide that is identical to the reference molecule, except for having one or more conservative amino acid substitutions in its amino acid sequence. In general, a conservatively modified variant includes an amino acid sequence that is at least about 70%, 75%, 80%, 85%, 90%, 91%, 92%, 93%, 94%, 95%, 96%, 97%, 98% or 99% identical to a reference amino acid sequence. More specifically, a conservative substitution refers to substitution of an amino acid with an amino acid having similar characteristics (e.g., charge, side-chain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “effective amount” means an amount or dose of one or more of the compounds herein, or a pharmaceutically acceptable salt thereof that, upon single or multiple dose administration to an individual in need thereof, provides a desired effect in such an individual under diagnosis or treatment (i.e., may produce a clinically measurable difference in a condition of the individual such as, for example, increased angiogenesis, increased vascular compliance, increased cardiac blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular compound administered, the mode of administration, the bioavailability characteristics of the preparation administered, the dose regimen selected, the use of concomitant medication, and other relevant circumstances.

As used herein, “extended duration of action” means that binding affinity and activity for a fusion including at least one compound herein and a biotherapeutic herein continues for a period of time greater than a native biotherapeutic, allowing for dosing at least as infrequently as once daily or even thrice-weekly, twice-weekly or once-weekly. The time action profile may be measured using known pharmacokinetic test methods such as those utilized in the Examples below.

As used herein, “glucocorticoid-induced TNFR-related protein” or “GITR”, also known as tumor necrosis factor receptor superfamily member 18 (TNFRSF18), means a GITR obtained or derived from any species, such as a mammalian species, especially a human. GITR includes both native GITR (i.e., full-length) and variations thereof (i.e., additions, deletions, insertions and/or substitutions of native GITR). One sequence for human GITR full-length (but without the signal peptide) is set forth in SEQ ID NO:122 (see also, UniProt/SwissProt Database Accession No. Q9Y5U5). One sequence for human GITR ECD (but without the signal peptide) is set forth in SEQ ID NO:123.

As used herein, “glucose-dependent insulinotropic peptide(s),” “gastric inhibitory peptide(s)” or “GIP(s)” means a GIP obtained or derived from any species, such as a mammalian species, especially a human. GIP includes both native GIP (i.e., full-length) and variations thereof (i.e., additions, deletions, insertions and/or substitutions of native GIP). GIP is processed from a precursor, proGIP. One sequence for proGIP is set forth in SEQ ID NO:67 (see also, UniProt/SwissProt Database Accession No. P09681) and one sequence for GIP is set forth in SEQ ID NO:68. An alternative GIP is GIP 1-30 (see, Hansen et al. (2016) Br. J. Pharmacol. 173:826-838). In humans, there is one GIP receptor (GIPR; SEQ ID NO:69; see also, UniProt/SwissProt Database Accession No. P48546), which acts as G protein-coupled receptor. See, Yaqub et al. (2010) Mol. Pharmacol. 77:547-558.

As used herein, “glucagon-like peptide-1” or “GLP-1” means a GLP-1 obtained or derived from any species, such as a mammalian species, especially a human. GLP-1 includes both native GLP-1 (i.e., full-length) and variations thereof (i.e., additions, deletions, insertions and/or substitutions of native GLP-1). GLP-1 is processed from a precursor, proglucagon (proGCG). One sequence for proGCG is set forth in SEQ ID NO:70 (see also, UniProt/SwissProt Database Accession No. P01275), and one sequence for GLP-1 is set forth in SEQ ID NO:71. Two active, physiological variants are known, which are set forth in SEQ ID NOS:72 and 73. In humans, there is one GLP-1 receptor (GLP-IR; SEQ ID NO:74; see also, UniProt/SwissProt Database Accession No. P43220), which acts as G protein-coupled receptor. See, Dillon et al. (1993) Endocrinol. 133:1907-1910; Graziano et al. (1993) Biochem. Biophys. Res. Commun. 196:141-146; and Thorens et al. (1993) Diabetes 42:1678-1682.

As used herein, “growth/differentiation factor 15” or “GDF15” means a GDF15 obtained or derived from any species, such as a mammalian species, especially a human. GDF15 includes both native GDF15 (i.e., full-length) and variations thereof (i.e., additions, deletions, insertions and/or substitutions of native GDF15). GDF15 is a homodimeric peptide that is processed from a precursor, proGDF15. One sequence for the precursor is set forth in SEQ ID NO: 75, and one sequence for GDF15 is set forth in SEQ ID NO: 76 (see also, UniProt/SwissProt Database Accession No. Q99988). In humans, there is one GDF15 receptor (GFRAL; SEQ ID NO:77; see also, UniProt/SwissProt Database Accession No. Q6UXV0), which acts as a RET-receptor tyrosine kinase. See, Emmerson et al. (2017) Nat Med. 23:1215-1219; Yang et al. (2017) Nat. Med. 23:1158-1166; and Baek & Eling (2019) Pharmacol. Ther. 198:46-58.

As used herein, “half-life” or “t½” means a time it takes for one-half of a quantity of a compound, such as a fusion described herein, to be removed from a fluid or other physiological space such as serum or plasma of an individual by biological processes. Alternatively, t½ also can mean a time it takes for a quantity of such a fusion to lose one-half of its pharmacological, physiological or radiological activity.

As used herein, “half-maximal effective concentration” or “EC₅₀” means a concentration of compound that results in 50% activation/stimulation of an assay endpoint, such as a dose-response curve (e.g., CNTF: Jak, STAT, Ras, PI3K/Akt and MAPK/ERK; NRG: PI3K/Akt, Jak, STAT, Ras and PLCy; GDF15: PI3K/AKT and MAPK/ERK and Smad; IL-2: JAK-STAT, PI3K/Akt and MAPK/ERK; GLP1: CAMP, PI3K, MAPK/ERK, PKC8; TNF: TRAF, MKK, IKK and NFKB; ACTH: CAMP and PKA).

As used herein, “in combination with” means administering at least one of the fusions herein either simultaneously, sequentially or in a single combined formulation with one or more additional therapeutic agents.

As used herein, “individual in need thereof” means a mammal, such as a human, with a condition, disease, disorder or symptom requiring treatment or therapy, including for example, those listed herein. In particular, the preferred individual to be treated is a human.

As used herein, “incretin(s)” or “INC(s)” means a peptide secreted from enteroendocrine cells that can increase insulin secretion following feeding. INC can be an incretin obtained or derived from any species, such as a mammalian species, especially a human. In humans, INCs include INS, GIP and GLP-1, which are discussed above.

As used herein, “insulin” or “INS” means an insulin obtained or derived from any species, such as a mammalian species, especially a human, where the native form is a heterodimeric peptide having two peptide chains (e.g., an A chain and a B chain) connected via two disulfide bonds, and with the A chain further having a single intramolecular disulfide bond. In humans, INS processing begins with preproinsulin (see, UniProt/SwissProt Database Accession No. P01308), which is processed to proinsulin (includes A chain, B chain and C peptide; native INS has a structure of B-C-A; see, SEQ ID NO:78; see also, UniProt/Swiss Prot Database Accession No. P01308). Proinsulin undergoes further processing in which the C peptide is cleaved to arrive at INS (see, SEQ ID NO: 79 for A chain of native, human INS and SEQ ID NO:80 for B chain of native, human INS; see also, UniProt/SwissProt Database Accession No. P01308).

As used herein, “interleukin(s)” or “IL(s)” means an interleukin obtained or derived from any species, such as a mammalian species, especially a human. IL includes both native IL (i.e., full-length) and variations thereof (i.e., additions, deletions, insertions and/or substitutions of native IL). In humans, there are a number of native IL isoforms; however, of interest herein is IL-2. IL-2 is a cytokine that can transduce signals via three different signaling pathways, which include JAK-STAT, PI3K/Akt/mTOR and MAPK/ERK pathways. One sequence for human IL-2 is set forth in SEQ ID NO:83 (see also, UniProt/SwissProt Database Accession No. P60568). In humans, there is one IL-2 receptor that includes α-, β- and γ-subunits (IL-2R; SEQ ID NOS:84 to 86; see also, UniProt/SwissProt Database Accession No. P01589, P14784 and P31785). See, Liao et al. (2011) Curr. Opin. Immunol. 23:598-604; and Malek & Castro (2010) Immunity 33:153-165.

As used herein, “long-acting” means that binding affinity and activity of a composition herein continues for a period of time greater than native peptide or protein, allowing for dosing at least as infrequently as once daily or even thrice-weekly, twice-weekly, once-weekly or monthly. The time action profile of the compounds herein may be measured using known pharmacokinetic test methods such as those described in the Examples below.

As used herein, “neuregulin(s)” or “NRG(s)” means a neuregulin obtained or derived from any species, such as a mammalian species, especially a human. NRG includes both native NRG (i.e., full-length) and variations thereof (i.e., additions, deletions, insertions and/or substitutions of native NRG). In humans, there are a number of native NRG family members; however, of interest herein is NRG1. Like all NRGs, NRG1 is processed from a larger precursor. One sequence for the precursor is set forth in SEQ ID NO:87, and one sequence for NRG1 is set forth in SEQ ID NO:88 (see also, UniProt/SwissProt Database Accession Nos. Q02297). In humans, there are two NRG1 receptors, ErbB3 (SEQ ID NO:89; see also, UniProt/SwissProt Database Accession No. P21860) and ErbB4 (SEQ ID NO:90; see also, UniProt/SwissProt Database Accession No. Q15303). See, Mei & Xiong (2008).

As used herein, “non-standard amino acid” means an amino acid that may occur naturally in cells but does not participate in peptide synthesis. Non-standard amino acids can be constituents of a peptide and oftentimes are generated by modification of standard amino acids in the peptide (i.e., via post-translational modification). Non-standard amino acids can include D-amino acids, which have an opposite absolute chirality of the standard amino acids above.

As used herein, “oligomer” means a molecule having a few similar or identical repeating units that could be derived from copies of a smaller molecule, its monomer. These monomers can be joined by bonds that are either strong or weak, covalent or non-covalent (e.g., intramolecular).

As used herein, “patient,” “subject” and “individual,” are used interchangeably herein, and mean a mammal, especially a human. In certain instances, the individual is further characterized with a condition, disease, disorder or symptom that would benefit from administering a compound or composition herein.

As used herein, “pharmaceutically acceptable buffer” means any of the standard pharmaceutical buffers known to one of skill in the art.

As used herein, “relaxin-2” or “RLN-2” means a relaxin-2 obtained or derived from any species, such as a mammalian species, especially a human, where the native form is a heterodimeric peptide having two peptide chains (e.g., an A chain and a B chain) connected via two disulfide bonds, and with the A chain further having a single intramolecular disulfide bond. In humans, RLN-2 processing begins with preprorelaxin (see, UniProt/SwissProt Database Accession No. P04090), which is processed to prorelaxin (includes A chain, B chain and C peptide; native RLN has a structure of B-C-A; see, SEQ ID NO:91; see also, UniProt/SwissProt Database Accession No. P04090). Prorelaxin undergoes further processing in which the C peptide is cleaved to arrive at RLN-2 (see, SEQ ID NO:92 for the A chain of RLN-2 and SEQ ID NO:93 for the B chain of RNL-2; see also, UniProt/SwissProt Database Accession No. P04090).

As used herein, “sequence similarity” means a quantitative property of two or more nucleic acid sequences or amino acid sequences of biological compounds such as, for example, a correspondence over an entire length or a comparison window of the two or more sequences. Sequence similarity can be measured by (1) percent identity or (2) percent similarity. Percent identity measures a percentage of residues identical between two biological compounds divided by the length of the shortest sequence, whereas percent similarity measures identities and, in addition, includes sequence gaps and residue similarity in the evaluation. Methods of and algorithms for determining sequence similarity are well known in the art and thus need not be exhaustively described herein. A specified percentage of identical nucleotide or amino acid positions is at least about 75%, 80%, 85%, 86%, 87%, 88%, 89%, 90%, 91%, 92%, 93%, 94%, 95%, 96%, 97%, 98%, 99% or higher.

As used herein, “treating” or “to treat” means managing and caring for an individual having a condition, disease, disorder or symptom for which administration of a compound herein is indicated for the purpose of attenuating, restraining, reversing, slowing or stopping progression or severity of the condition, disease, disorder or symptom. Treating includes administering a compound herein or composition containing a compound herein to the individual to prevent the onset of symptoms or complications, alleviating the symptoms or complications, or eliminating the condition, disease, disorder or symptom. Treating includes administering a compound herein or composition containing a compound herein to the individual to result in such as, for example, increased angiogenesis, increased vascular compliance, increased cardiac blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung. The individual to be treated is a mammal, especially a human.

As used herein, “VHH” or “VHH moiety” means a form of single-domain antibody, especially an antibody fragment comprising a single, monomeric variable region of a heavy chain only antibody (HcAb), which may have a size of about 15 kDa. It has been found herein that engineered/modified VHH-based compounds can be used as a pharmacokinetic enhancer to extend the duration of action of and/or to improve the t½ of biotherapeutics. The VHH-based compounds bind serum albumin; however, the VHH-based compounds can be used to bind IgG (including Fc domain), neonatal Fc receptor (FcRn) or other long-lasting serum proteins. The VHH-based compound therefore can be used to improve the t½ of a compound such as a peptide or protein or even other molecules such as, for example, small molecules.

Certain abbreviations are defined as follows: “ACR” refers to urine albumin/urine creatinine ratio; “AUC” refers to area under the curve; “CAMP” refers to cyclic adenosine monophosphate; “CMV” refers to cytomegalovirus; “DNA” refers to deoxyribonucleic acid; “ECD” refers to extracellular domain; “EDC” refers to 1-ethyl-3-(3-dimethylaminopropyl) carbodiimide hydrochloride; “ETA” refers to ethanolamine; “GS” refers to glutamine synthetase; “HC” refers to heavy chain; “HIC” refers to hydrophobic interaction chromatography; “hr” refers to hour or hours; “IV” refers to intravenous; “kDa” refers to kilodaltons; “LC” refers to light chain; “LC-MS” refers to liquid chromatography-mass spectrometry; “min” refers to minute or minutes; “MS” refers to mass spectrometry; “MSX” refers to methionine sulfoximine; “NHS” refers to N-hydroxysuccinimide; “OtBu” refers to O-tert-butyl; “PEI” refers to polyethylenimine; “RP-HPLC” refers to reversed-phase high performance liquid chromatography; “sec” refers to second or seconds; “NaOAc” refers to sodium acetate; “rcf” means relative centrifugal force; “RT” means room temperature; “RU” means resonance units; “SQ” refers to subcutaneous; “SEC” refers to size-exclusion chromatography; “SEM” refers to standard error of the mean; “SPR” means surface plasmon resonance; “TFA” refers to trifluoroacetic acid; and “Trt” refers to trityl.

### VHH-Based Compounds Acting as Half-Life Extenders and Fusion or Conjugated Compounds Including the Same

Briefly, the compounds herein can include an amino acid sequence from the N-terminus to the C-terminus having one of the following structures:
M-X₁,
X₁-M,
M-X₂,
X₂-M,
M-L₁-X₁,
M-L₂-X₁,
M-L₁-X₂,
M-L₂-X₂,
X₁-L₁-M,
X₁-L₂-M,
X₂-L₁-M,
X₂-L₂-M,
X₁-M-X₂,
X₂-M-X₁,
X₁-L₁-M-X₂,
X₂-L₁-M-X₁,
X₁-M-L₁-X₂,
X₂-M-L₁-X₁,
X₁-L₁-M-L₂-X₂,
X₂-L₁-M-L₂-X₁,
M-L₁-X₁-L₂-X₂,
M-L₁-X₂-L₂-X₁,
X₁-L₂-X₂-L₁-M,
X₂-L₂-X₁-L₁-M, or
M-L₁-X₁ combined with M-L₂-X₂ (i.e., non-covalently associated),
where M is a VHH-based compound acting as a t½-extending moiety and having an amino acid sequence selected from SEQ ID NOS:1 to 37 and 124 to 126 or having an amino acid sequence having at least about 90% to about 99% sequence similarity thereto, where L₁ (if present) is a first linker, where L₂ (if present) is a second linker, where X₁ (if present) is a biotherapeutic protein, peptide or oligomer, and where X₂ (if present) likewise is a biotherapeutic protein, peptide, or oligomer that can be the same as or distinct from X₁ (such as when the biotherapeutic is a homodimer, or when the biotherapeutic is a heterodimer X₁ can be one chain (a-chain) and X₂ can be another chain (β-chain) thereof). X₁ and X₂ can also be fully distinct from one another.

In some instances, L₁ can have an amino acid sequence of (GGGGQ)ₙ (SEQ ID NO:38), (GGGQ)ₙ (SEQ ID NO:39), (GGGGS)ₙ (SEQ ID NO:40), (PGPQ)ₙ (SEQ ID NO:40), (PGPA)ₙ (SEQ ID NO:42), GGGG(AP)ₙGGGG (SEQ ID NO:43), (GGE)ₙ (SEQ ID NO:44), (GGGGE)ₙ (SEQ ID NO:45), (GGK)ₙ (SEQ ID NO:46), (GGGGK)ₙ (SEQ ID NO:47), GGGG(EP)ₙGGGG (SEQ ID NO:48), GGGG(KP)ₙGGGG (SEQ ID NO:49), (PGPE)ₙ (SEQ ID NO:50), or (PGPK)ₙ (SEQ ID NO:51), where n can be from 1 to 15, especially from about 5 to about 10. In other instances, L₁ can have an amino acid sequence selected from any one of SEQ ID NOS:52 to 63. In still other instances, L₁ can have one or more additions, deletions, insertions or substitutions such that L₁ has an amino acid sequence having at least about 90% to about 99% sequence similarity to any one of SEQ ID NOS:52 to 63.

In some instances, L₂ can have an amino acid sequence selected from any one of SEQ ID NOS:64 to 65. In other instances, L₂ can have one or more additions, deletions, insertions or substitutions such that L₂ has an amino acid sequence having at least about 90% to about 99% sequence similarity to any one of SEQ ID NOS:64 to 65.

In still other instances, L₁ or L₂ can be a polymer such as a polyethylene glycol (PEG), especially maleimide-(PEG)₁₂.

Taken together, exemplary fusions are as follows:

Compound 1, which is a VHH-CNTF fusion that includes CNTF, a (G₄Q)₅ linker (italicized) and a VHH-based compound acting as a t½-extending moiety (underlined), has the following amino acid sequence:

(SEQ ID NO: 100)
MAFTEHSPLTPHRRDLASRSIWLARKIRSDLTALTESYVKHQGLNKN
INLDSADGMPVASTDRWSELTEAERLQENLQAYRTFHVLLARLLEDQ
QVHFTPTEGDFHQAIHTLLLQVAAFAYQIEELMILLEYKIPRNEADG
MPINVGDGGLFEKKLWGLKVLQELSQWTVRSIHDLRFISSHQTGGGG
GQGGGGQGGGGQGGGGQGGGGQEVQLLESGGGLVQPGGSLRLSCAAS
GRYIDETAVAWFRQAPGKEREFVAGIGGGVDITYYADSVKGRFTISR
DNSKNTLYLQMNSLRPEDTAVYYCAARPGRPLITSKVADLYPYWGQG
TLVTVSSPP.

Compound 2, which is a VHH-CNTF fusion that includes a VHH-based compound acting as a t½-extending moiety (underlined), a (G₄Q)₅ linker (italicized) and CNTF, has the following amino acid sequence:

(SEQ ID NO: 101)
EVQLLESGGGLVQPGGSLRLSCAASGRYIDETAVAWFRQAPGKEREF
VAGIGGGVDITYYADSVKGRFTISRDNSKNTLYLQMNSLRPEDTAVY
YCAARPGRPLITSKVADLYPYWGQGTLVTVSSGGGGQGGGGQGGGGQ
GGGGQGGGGQMAFTEHSPLTPHRRDLASRSIWLARKIRSDLTALTES
YVKHQGLNKNINLDSADGMPVASTDRWSELTEAERLQENLQAYRTFH
VLLARLLEDQQVHFTPTEGDFHQAIHTLLLQVAAFAYQIEELMILLE
YKIPRNEADGMPINVGDGGLFEKKLWGLKVLQELSQWTVRSIHDLRF
ISSHQTG.

Compound 3, which is a VHH-NRG1 fusion that includes NRG1, a (G₄Q)₅ linker (italicized) and a VHH-based compound acting as a t½-extending moiety (underlined), has the following amino acid sequence:

(SEQ ID NO: 102)
SHLVKCAEKEKTFCVNGGECFMVKDLSNPSRYLCKCPNEFTGDRCQN
YVMASFYKHLGIEFMEAEELYQGGGGQGGGGQGGGGQGGGGQGGGGQ
EVQLLESGGGLVQPGGSLRLSCAASGRYIDETAVAWFRQAPGKEREF
VAGIGGGVDITYYADSVKGRFTISRDNSKNTLYLQMNSLRPEDTAVY
YCAARPGRPLITSKVADLYPYWGQGTLVTVSSPP.

Compound 4, which is a VHH-NRG1 fusion that includes a VHH-based compound acting as a t½-extending moiety (underlined), a (G₄Q)₅ linker (italicized) and NRG1, has the following amino acid sequence:

(SEQ ID NO: 103)
EVQLLESGGGLVQPGGSLRLSCAASGRYIDETAVAWFRQAPGKEREF
VAGIGGGVDITYYADSVKGRFTISRDNSKNTLYLQMNSLRPEDTAVY
YCAARPGRPLITSKVADLYPYWGQGTLVTVSSGGGGQGGGGQGGGGQ
GGGGQGGGGQSHLVKCAEKEKTFCVNGGECFMVKDLSNPSRYLCKCP
NEFTGDRCQNYVMASFYKHLGIEFMEAEELYQ.

Compound 5, which is a VHH-GDF15 fusion that includes a VHH-based compound acting as a t½-extending moiety (underlined), a (G₄Q)₅ linker (italicized) and GDF15, has the following amino acid sequence:

(SEQ ID NO: 104)
EVQLLESGGGLVQPGGSLRLSCAASGRYIDETAVAWFRQAPGKEREFVA
GIGGGVDITYYADSVKGRFTISRDNSKNTLYLQMNSLRPEDTAVYYCAA
RPGRPLITSKVADLYPYWGQGTLVTVSSGGGGQGGGGQGGGGQGGGGQG
GGGQGDHCPLGPGRCCRLHTVRASLEDLGWADWVLSPREVQVTMCIGAC
PSQFRAANMHAQIKTSLHRLKPDTVPAPCCVPASYNPMVLIQKTDTGVS
LQTYDDLLAKDCHCI.

Compound 6, which is a VHH-IL-2 fusion that includes IL-2, a (G₄Q)₅ linker (italicized) and a VHH-based compound acting as a t½-extending moiety (underlined), has the following amino acid sequence:

(SEQ ID NO: 105)
APQSSSTQQTQLQLEHLLLDLQMILNGINNYKNPKLTRMLTFKFYMPKK
ATELKHLQCLEEELKPLEEVLNLAQSKNFHLRPRDLISRINVIVLELKG
SETTFMCEYADETATIVEFLNRWITFAQSIISTLTGGGGQGGGGQGGGG
QGGGGQGGGGQEVQLLESGGGLVQPGGSLRLSCAASGRYIDETAVAWFR
QAPGKEREFVAGIGGGVDITYYADSVKGRFTISRDNSKNTLYLQMNSLR
PEDTAVYYCAARPGRPLITSKVADLYPYWGQGTLVTVSSPP.

Compound 7, which is a VHH-IL-2 fusion that includes a VHH-based compound acting as a t½-extending moiety (underlined), a (G₄Q)₅ linker (italicized) and IL-2, has the following amino acid sequence:

(SEQ ID NO: 106)
EVQLLESGGGLVQPGGSLRLSCAASGRYIDETAVAWFRQAPGKEREFVA
GIGGGVDITYYADSVKGRFTISRDNSKNTLYLQMNSLRPEDTAVYYCAA
RPGRPLITSKVADLYPYWGQGTLVTVSSGGGGQGGGGQGGGGQGGGGQG
GGGQAPQSSSTQQTQLQLEHLLLDLQMILNGINNYKNPKLTRMLTFKFY
MPKKATELKHLQCLEEELKPLEEVLNLAQSKNFHLRPRDLISRINVIVL
ELKGSETTFMCEYADETATIVEFLNRWITFAQSIISTLT.

Compound 8, which is a VHH-GLP-1 fusion that includes GLP-1, a (G₄Q)₅ linker (italicized) and a VHH-based compound acting as a t½-extending moiety (underlined), has the following amino acid sequence:

(SEQ ID NO: 107)
HGEGTFTSDVSSYLEEQAAKEFIAWLVKGGGGGGGQGGGGQGGGGQGGG
GQGGGGQEVQLLESGGGLVQPGGSLRLSCAASGRYIDETAVAWFRQAPG
KEREFVAGIGGGVDITYYADSVKGRFTISRDNSKNTLYLQMNSLRPEDT
AVYYCAARPGRPLITSKVADLYPYWGQGTLVTVSSPP.

Compound 9, which is a VHH-Fab fusion that includes a HC, a (G₄Q)₅ linker (italicized) and a VHH-based compound acting as a t½-extending moiety (underlined), has the following amino acid sequence:

(SEQ ID NO: 108)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTGGGGQGGGGQGGGGQG
GGGQGGGGQEVQLLESGGGLVQPGGSLRLSCAASGRYIDETAVAWFRQA
PGKEREFVAGIGGGVDITYYADSVKGRFTISRDNSKNTLYLQMNSLRPE
DTAVYYCAARPGRPLITSKVADLYPYWGQGTLVTVSSPP.

Compound 9 further includes a LC having the following amino acid sequence:

(SEQ ID NO: 109)
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Compound 10, which is a VHH-Fab fusion that includes a LC, a (G₄Q)₅ linker (italicized) and a VHH-based compound acting as a t½-extending moiety (underlined), has the following amino acid sequence:

(SEQ ID NO: 111)
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECGGGGQGGGGQGGGGQGGGGQGGGGQEVQLLE
SGGGLVQPGGSLRLSCAASGRYIDETAVAWFRQAPGKEREFVAGIGGGV
DITYYADSVKGRFTISRDNSKNTLYLQMNSLRPEDTAVYYCAARPGRPL
ITSKVADLYPYWGQGTLVTVSSPP.

Compound 10 further includes a HC having the following amino acid sequence:

(SEQ ID NO: 110)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCD.

Compound 11, which is a VHH-Fab fusion that includes a VHH-based compound acting as a t½-extending moiety (underlined), a (G₄Q)₅ linker (italicized) and a HC, has the following amino acid sequence:

(SEQ ID NO: 112)
EVQLLESGGGLVQPGGSLRLSCAASGRYIDETAVAWFRQAPGKEREFVA
GIGGGVDITYYADSVKGRFTISRDNSKNTLYLQMNSLRPEDTAVYYCAA
RPGRPLITSKVADLYPYWGQGTLVTVSSGGGGQGGGGQGGGGQGGGGQG
GGGQEVQLVESGGGLVQPGRSLRLSCAASGFTFDDYAMHWVRQAPGKGL
EWVSAITWNSGHIDYADSVEGRFTISRDNAKNSLYLQMNSLRAEDTAVY
YCAKVSYLSTASSLDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCD.

Compound 11 further includes a LC having the following amino acid sequence:

(SEQ ID NO: 113)
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Compound 12, which is a VHH-Fab fusion that includes a VHH-based compound acting as a t½-extending moiety (underlined), a (G₄Q)₅ linker (italicized) and a LC, has the following amino acid sequence:

(SEQ ID NO: 115)
EVQLLESGGGLVQPGGSLRLSCAASGRYIDETAVAWFRQAPGKEREFVA
GIGGGVDITYYADSVKGRFTISRDNSKNTLYLQMNSLRPEDTAVYYCAA
RPGRPLITSKVADLYPYWGQGTLVTVSSGGGGQGGGGGGGGQGGGGQGG
GGQDIQMTQSPSSLSASVGDRVTITCRASQGIRNYLAWYQQKPGKAPKL
LIYAASTLQSGVPSRFSGSGSGTDFTLTISSLQPEDVATYYCQRYNRAP
YTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC.

Compound 12 further includes a HC having the following amino acid sequence:

(SEQ ID NO: 114)
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCD.

Compound 13, which is a VHH-GLP-1 fusion that includes GLP-1, a (G₄Q)₅ linker (italicized), a VHH-based compound acting as a t½-extending moiety (underlined) and a C-terminal Cys, has the following amino acid sequence: HGEGTFTSDVSSYLEEQAAKEFIAWLVKGGGGGGGQGGGGQGGGGQGGGGQG

(SEQ ID NO: 116)
GGGQEVQLLESGGGLVQPGGSLRLSCAASGRYIDETAVAWFRQAPGKGR
EFVAGIGGGVDITYYADSVKGRFTISRDNSKNTLYLQMNSLRPEDTAVY
YCAARPGRPLITSKVADLYPYWGQGTLVTVSSC.

Compound 14, which is a VHH-GLP-1/ACTH fusion conjugate that includes GLP-1, a (G₄Q)₅ linker (italicized), a VHH-based compound acting as a t½-extending moiety (underlined), a C-terminal Cys and ACTH connected in a C-terminal to N-terminal orientation via a maleimide(PEG)₁₂ linker, has the following amino acid sequence:

(SEQ ID NO: 117)
HGEGTFTSDVSSYLEEQAAKEFIAWLVKGGGGGGGQGGGGQGGGGQGGG
GQGGGGQEVQLLESGGGLVQPGGSLRLSCAASGRYIDETAVAWFRQAPG
KGREFVAGIGGGVDITYYADSVKGRFTISRDNSKNTLYLQMNSLRPEDT
AVYYCAARPGRPLITSKVADLYPYWGQGTLVTVSSC-maleimide-
(PEG)₁₂-ACTH (in a C-terminal to N-terminal
orientation).

Compound 15, which is a VHH-ACTH fusion conjugate that includes a VHH-based compound acting as a t½-extending moiety (underlined), a C-terminal Cys and ACTH connected in a C-terminal to N-terminal orientation via a maleimide(PEG)₁₂ linker, has the following amino acid sequence:

(SEQ ID NO: 118)
EVQLLESGGGLVQPGGSLRLSCAASGRYIDETAVAWFRQAPGKGREFVA
GIGGGVDITYYADSVKGRFTISRDNSKNTLYLQMNSLRPEDTAVYYCAA
RPGRPLITSKVADLYPYWGQGTLVTVSSC-maleimide-(PEG)₁₂-
(ACTH in a C-terminal to N-terminal orientation).

### Pharmaceutical Compositions and Kits

The compounds (i.e., VHH-based fusions or VHH-based conjugates such as, for example, Compounds 1 to 15 above) herein can be formulated as pharmaceutical compositions, which can be administered by parenteral routes (e.g., intravenous, intraperitoneal, intramuscular, subcutaneous or transdermal). Such pharmaceutical compositions and techniques for preparing the same are well known in the art. See, e.g., Remington, “The Science and Practice of Pharmacy” (D. B. Troy ed., 21^{st} Ed., Lippincott, Williams & Wilkins, 2006). In particular instances, the compositions are administered SQ or IV. Alternatively, however, the compositions can be formulated in forms for other pharmaceutically acceptable routes such as, for example, tablets or other solids for oral administration; time release capsules, and any other form currently used, including creams, lotions, inhalants and the like.

As noted above, and to improve their in vivo compatibility and effectiveness, the VHH-based fusions or VHH-based conjugates herein may be reacted with any number of inorganic and organic acids/bases to form pharmaceutically acceptable acid/base addition salts. Pharmaceutically acceptable salts and common techniques for preparing them are well known in the art (see, e.g., Stahl et al., “Handbook of Pharmaceutical Salts: Properties, Selection and Use” (2nd Revised Ed. Wiley-VCH, 2011)). Pharmaceutically acceptable salts for use herein include sodium, trifluoroacetate, hydrochloride and acetate salts.

The compounds herein may be administered by a physician or self-administered using an injection. It is understood the gauge size and amount of injection volume can be readily determined by one of skill in the art. However, the amount of injection volume can be ≤about 2 mL or even ≤about 1 mL, and the needle gauge can be ≥about 27 G or even ≥about 29 G.

The disclosure also provides and therefore encompasses novel intermediates and methods useful for synthesizing the compounds herein, or a pharmaceutically acceptable salt thereof. The intermediates and compounds can be prepared by a variety of techniques that are well known in the art. For example, a method using recombinant synthesis is illustrated in the Examples below. The specific steps for each of the techniques described may be combined in different ways to prepare the compounds. The reagents and starting materials are readily available to one of skill in the art.

The compounds herein are generally effective over a wide dosage range. Exemplary doses of the compounds or of pharmaceutical compositions including the same can be milligram (mg) or microgram (μg), nanogram (ng), or picogram (pg) amounts per kilogram (kg) of an individual. In this manner, a daily dose can be from about 1 μg to about 100 mg.

Here, the effective amount of the compound in a pharmaceutical composition can be a dose of about 0.25 mg to about 5.0 mg. One of skill in the art, however, understands that in some instances the effective amount (i.e., dose/dosage) may be below the lower limit of the aforesaid range and be more than adequate, while in other cases the effective amount may be a larger dose and may be employed with acceptable side effects.

In addition to a compound herein, the pharmaceutical composition also can include at least one additional therapeutic agent such as, for example, a therapeutic agent typically used as the standard of care in of a particular condition, disease and disorder (e.g., a cardiovascular, neurological, immunological, metabolic, oncological, psychological, pulmonary and/or renal condition, disease or disorder).

In this manner, a pharmaceutical composition can include an effective amount of one or more compounds herein, a pharmaceutically acceptable carrier and optionally at least one additional therapeutic agent. For example, the pharmaceutical composition can include an effective amount of a compound of SEQ ID NO:100 and a pharmaceutically acceptable carrier, an effective amount of a compound of SEQ ID NO:101 and a pharmaceutically acceptable carrier, an effective amount of a compound of SEQ ID NO:102 and a pharmaceutically acceptable carrier, an effective amount of a compound of SEQ ID NO:103 and a pharmaceutically acceptable carrier, an effective amount of a compound of SEQ ID NO: 104 and a pharmaceutically acceptable carrier, an effective amount of a compound of SEQ ID NO:105 and a pharmaceutically acceptable carrier, an effective amount of a compound of SEQ ID NO: 106 and a pharmaceutically acceptable carrier, an effective amount of a compound of SEQ ID NO:107 and a pharmaceutically acceptable carrier, an effective amount of a compound of SEQ ID NOS: 108 and 109 and a pharmaceutically acceptable carrier, an effective amount of a compound of SEQ ID NOS:110 and 111 and a pharmaceutically acceptable carrier, an effective amount of a compound of SEQ ID NOS: 112 and 113 and a pharmaceutically acceptable carrier, an effective amount of a compound of SEQ ID NOS:114 and 115 and a pharmaceutically acceptable carrier, an effective amount of a compound of SEQ ID NO:116 and a pharmaceutically acceptable carrier, an effective amount of a compound of SEQ ID NO:117 and a pharmaceutically acceptable carrier, and an effective amount of a compound of SEQ ID NO: 118 and a pharmaceutically acceptable carrier.

Alternatively, the compounds herein can be provided as part of a kit. In some instances, the kit includes a device for administering at least one compound (and optionally at least one additional therapeutic agent) to an individual. In certain instances, the kit includes a syringe and needle for administering the at least one compound (and optionally at least one additional therapeutic agent). In particular instances, the compound (and optionally at least one additional therapeutic agent) is pre-formulated in aqueous solution within the syringe.

### Methods of Making and Using VHH-Based Compounds Acting as Half-Life Extenders or Fusions and Conjugates Thereof

The compounds herein can be made via any number of standard recombinant DNA methods or standard chemical peptide synthesis methods known in the art. With regard to recombinant DNA methods, one can use standard recombinant techniques to construct a polynucleotide having a nucleic acid sequence that encodes an amino acid sequence for a compound (i.e., fusion peptide or fusion protein or fusion conjugate), incorporate that polynucleotide into recombinant expression vectors, and introduce the vectors into host cells, such as bacteria, yeast and mammalian cells, to produce the compound. See, e.g., Green & Sambrook, “Molecular Cloning: A Laboratory Manual” (Cold Spring Harbor Laboratory Press, 4^{th} ed. 2012).

With regard to recombinant DNA methods, the compounds herein can be prepared by producing a protein or precursor protein molecule using recombinant DNA techniques. DNA, including cDNA and synthetic DNA, may be double-stranded or single-stranded, and the coding sequences therein encoding a compound herein may vary as a result of the redundancy or degeneracy of the genetic code. Briefly, the DNA sequences encoding the compounds herein are introduced into a host cell to produce the compound or precursor thereof. The host cells can be bacterial cells such as K12 or B strains of Escherichia coli, fungal cells such as yeast cells, or mammalian cells such as Chinese hamster ovary (CHO) cells.

An appropriate host cell is transiently or stably transfected or transformed with an expression system, such as expression vectors, for producing a compound herein or a precursor thereof. Expression vectors typically are replicable in the host organisms either as episomes or as an integral part of the host chromosomal DNA. Commonly, expression vectors will contain selection markers such as, for example, tetracycline, neomycin, G₄₁₈ and dihydrofolate reductase, to permit selection of those cells transformed with the desired DNA sequences.

The specific biosynthetic or synthetic steps for each of the steps described herein may be used, not used or combined in different ways to prepare the compounds herein.

With regard to chemical peptide synthesis methods, one can use standard manual or automated solid-phase synthesis procedures. For example, automated peptide synthesizers are commercially available from, for example, Applied Biosystems (Foster City, CA) and Protein Technologies Inc. (Tucson, AZ). Reagents for solid-phase synthesis are readily available from commercial sources. Solid-phase synthesizers can be used according to the manufacturer's instructions for blocking interfering groups, protecting amino acids during reaction, coupling, deprotecting and capping of unreacted amino acids.

One use for the compounds herein is in treating cardiovascular, neurological, immunological, metabolic, oncological, psychological, pulmonary and/or renal condition, disease or disorder.

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Such methods therefore can include selecting an individual having, for example, a neurological condition, disease or disorder, or who is predisposed to the same. Alternatively, the methods can include selecting an individual having a metabolic condition, disease or disorder, or who is predisposed to the same. Alternatively, the methods can include selecting an individual having a cardiovascular condition, disease or disorder, or who is predisposed to the same. Alternatively, the methods can include selecting an individual having an oncology condition, disease or disorder, or who is predisposed to the same. Alternatively, the methods can include selecting an individual having a psychological condition, disease or disorder, or who is predisposed to the same. Alternatively, the methods can include selecting an individual having a pulmonary condition, disease or disorder, or who is predisposed to the same. Alternatively, the methods can include selecting an individual having a renal condition, disease or disorder, or who is predisposed to the same. Alternatively, the methods can include selecting an individual having an autoimmune condition, disease or disorder, or who is predisposed to the same.

The methods also can include administering to the individual an effective amount of at least one compound herein, which may be in the form of a pharmaceutical composition as also described herein. In some instances, the compound/pharmaceutical composition can include an additional therapeutic agent.

The concentration/dose/dosage of the compound and optional additional therapeutic agent are discussed elsewhere herein.

With regard to a route of administration, the compound or pharmaceutical composition including the same can be administered in accord with known methods such as, for example, orally; by injection (i.e., intra-arterially, intravenously, intraperitoneally, intracerebrally, intracerebroventricularly, intramuscularly, intraocularly, intraportally or intralesionally); by sustained release systems, or by implantation devices. In certain instances, the compound or pharmaceutical composition including the same can be administered SQ by bolus injection or continuously.

With regard to a dosing frequency, the compound or pharmaceutical composition including the same can be administered daily, every other day, three times a week, two times a week, one time a week (i.e., weekly), biweekly (i.e., every other week), or monthly. In certain instances, the compound or pharmaceutical composition including the same is administered SQ every other day, SQ three times a week, SQ two times a week, SQ one time a week, SQ every other week or SQ monthly. In particular instances, the compound or pharmaceutical composition including the same is administered SQ one time a week (QW).

Alternatively, and if administered IV, the compound or pharmaceutical composition including the same is administered IV every other day, IV three times a week, IV two times a week, IV one time a week, IV every other week or IV monthly. In particular instances, the compound or pharmaceutical composition including the same is administered IV one time a week (IW).

With regard to those instances in which the compound or pharmaceutical composition including the same is administered in combination with an effective amount of at least one additional therapeutic agent, the additional therapeutic agent can be administered simultaneously, separately or sequentially with the compound or pharmaceutical composition including the same.

Moreover, the additional therapeutic agent can be administered with a frequency same as the compound or pharmaceutical composition including the same (i.e., every other day, twice a week, or even weekly). Alternatively, the additional therapeutic agent can be administered with a frequency distinct from the compound or pharmaceutical composition including the same. In other instances, the additional therapeutic agent can be administered SQ. In other instances, the additional therapeutic agent can be administered IV. In still other instances, the additional therapeutic agent can be administered orally.

It is further contemplated that the methods may be combined with diet and exercise and/or may be combined with additional therapeutic agents other than those discussed above.

### EXAMPLES

The following non-limiting examples are offered for purposes of illustration, not limitation.

### Polypeptide Expression, Purification and Conjugation

### Example 1: Recombinant Expression and Purification of VHH-Based Fusion 1

Example 1 is a CNTF-VHH fusion having an amino acid sequence of:

(SEQ ID NO: 100)
MAFTEHSPLTPHRRDLASRSIWLARKIRSDLTALTESYVKHQGLN
KNINLDSADGMPVASTDRWSELTEAERLQENLQAYRTFHVLLARL
LEDQQVHFTPTEGDFHQAIHTLLLQVAAFAYQIEELMILLEYKIP
RNEADGMPINVGDGGLFEKKLWGLKVLQELSQWTVRSIHDLRFIS
SHQTGGGGGQGGGGQGGGGQGGGGQGGGGQEVQLLESGGGLVQPG
GSLRLSCAASGRYIDETAVAWFRQAPGKEREFVAGIGGGVDITYY
ADSVKGRFTISRDNSKNTLYLQMNSLRPEDTAVYYCAARPGRPLI
TSKVADLYPYWGQGTLVTVSSPP.

Here, the VHH fusion of SEQ ID NO:100 is generated in a mammalian cell expression system using CHOK1 cell derivatives. A cDNA sequence encoding SEQ ID NO:100 is sub-cloned into GS-containing expression plasmid backbone (pEE12.4-based plasmids). The cDNA sequence is fused in frame with the coding sequence of a signal peptide sequence, METDTLLLWVLLLWVPGSTG (SEQ ID NO:66) to enhance secretion of the VHH fusion analog into the tissue culture medium. The expression is driven by the viral CMV promoter.

For generating the VHH fusion via transient transfection, CHOK1 cells are transfected with the recombinant expression plasmid using a PEI-based method. Briefly, the appropriate volume of CHOK1 suspension cells at a density of 4×10⁶ cells/mL is transferred in shake flasks, and both PEI and recombinant plasmid DNA are added to the cells. Cells are incubated in a suspension culture at 32° C. for 6 days. At the end of the incubation period, cells are removed by low-speed centrifugation and the VHH fusion is purified from the conditioned medium.

Alternatively, and for generating the VHH fusion via stable transfections, CHOK1 cells are stably transfected using electroporation and an appropriate amount of recombinant expression plasmid, and the transfected cells are maintained in suspension culture at an adequate cell density. Selection of the transfected cells is accomplished by growth in 25 μM MSX-containing serum-free medium and incubated at about 37° C. and about 6% CO₂.

The VHH fusion secreted into the media from the CHO cells is purified by Protein A affinity chromatography followed by ion exchange, hydrophobic interaction, or size-exclusion chromatography. Specifically, the VHH fusion from harvested media is captured onto Mab Select Protein A resin (GE Healthcare). The resin then is briefly washed with a running buffer, such as a phosphate-buffered saline (PBS; pH 7.4) or a running buffer containing Tris, to remove non-specifically bound material. The VHH fusion is eluted from the resin with a low pH solution, such as 10 mM citric acid pH 3. Fractions containing the VHH fusion are pooled and may be held at a low pH to inactivate potential viruses. The pH may be neutralized by adding a base such as 1M Tris pH 8.0. The VHH fusion may be further purified by ion exchange chromatography using resins such as Poros 50 HS (ThermoFisher). The VHH fusion is eluted from the ion exchange column using a 0 to 500 mM NaCl gradient in 20 mM NaOAc, pH 5.0 over 15 column volumes.

The VHH fusion may be further purified by hydrophobic interaction chromatography by using a Capto Phenyl ImpRes HIC Column (GE Healthcare). The purification is performed by adjusting the column charge solution to around 0.5 M sodium sulfate and eluting using a 10 Column Volume (CV) gradient going from 0.5 M to 0 M sodium sulfate in a 20 mM Tris pH 8 solution. After HIC, the VHH fusion may be even further purified by SEC by loading the concentrated Capto Phenyl ImpRes pool on a Superdex200 or Superdex75 (GE Healthcare) with isocratic elution in PBS pH 7.4 or in 20 mM histidine, 50 mM NaCl pH6.0.

Purified VHH fusion may be passed through a viral retention filter such as Planova 20N (Asahi Kasei Medical) followed by concentration/diafiltration into 20 mM histidine, 20 mM NaCl pH 6 using tangential flow ultrafiltration on a regenerated cellulose membrane (Millipore).

The VHH fusion therefore is prepared in this manner or in a similar manner that would be readily determined by one of skill in the art.

### Example 2: Recombinant Expression and Purification of VHH-Based Fusion 2

Example 2 is a VHH-CNTF fusion having an amino acid sequence of:

(SEQ ID NO: 101)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQMAFTEHSPLTPHRRDLASRSIWLARKIRS
DLTALTESYVKHQGLNKNINLDSADGMPVASTDRWSELTEAERLQ
ENLQAYRTFHVLLARLLEDQQVHFTPTEGDFHQAIHTLLLQVAAF
AYQIEELMILLEYKIPRNEADGMPINVGDGGLFEKKLWGLKVLQE
LSQWTVRSIHDLRFISSHQTG.

Here, the VHH fusion of SEQ ID NO:101 is generated essentially as described for Example 1 except that a cDNA sequence encoding SEQ ID NO:101 is used in the expression plasmid.

### Example 3: Recombinant Expression and Purification of VHH-Based Fusion 3

Example 3 is a NRG1-VHH fusion having an amino acid sequence of:

(SEQ ID NO: 102)
SHLVKCAEKEKTFCVNGGECFMVKDLSNPSRYLCKCPNEFTGDRC
QNYVMASFYKHLGIEFMEAEELYQGGGGQGGGGQGGGGQGGGGQG
GGGQEVQLLESGGGLVQPGGSLRLSCAASGRYIDETAVAWFRQAP
GKEREFVAGIGGGVDITYYADSVKGRFTISRDNSKNTLYLQMNSL
RPEDTAVYYCAARPGRPLITSKVADLYPYWGQGTLVTVSSPP.

Here, the VHH fusion of SEQ ID NO:102 is generated essentially as described for Example 1 except that a cDNA sequence encoding SEQ ID NO: 102 is used in the expression plasmid.

### Example 4: Recombinant Expression and Purification of VHH-Based Fusion 4

Example 4 is a VHH-NRG1 fusion having an amino acid sequence of:

(SEQ ID NO: 103)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSHLVKCAEKEKTFCVNGGECFMVKDLSNP
SRYLCKCPNEFTGDRCQNYVMASFYKHLGIEFMEAEELYQ.

Here, the VHH fusion of SEQ ID NO:103 is generated essentially as described for Example 1 except that a cDNA sequence encoding SEQ ID NO:103 is used in the expression plasmid.

### Example 5: Recombinant Expression and Purification of VHH-Based Fusion 5

Example 5 is a VHH-GDF15 fusion having an amino acid sequence of:

(SEQ ID NO: 104)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQGDHCPLGPGRCCRLHTVRASLEDLGWADW
VLSPREVQVTMCIGACPSQFRAANMHAQIKTSLHRLKPDTVPAPC
CVPASYNPMVLIQKTDTGVSLQTYDDLLAKDCHCI.

Here, the VHH fusion of SEQ ID NO:104 is generated essentially as described for Example 1 except that a cDNA sequence encoding SEQ ID NO:104 is used in the expression plasmid.

### Example 6: Recombinant Expression and Purification of VHH-Based Fusion 6

Example 6 is an IL-2-VHH fusion having an amino acid sequence of:

(SEQ ID NO: 105)
APQSSSTQQTQLQLEHLLLDLQMILNGINNYKNPKLTRMLTFKFY
MPKKATELKHLQCLEEELKPLEEVLNLAQSKNFHLRPRDLISRIN
VIVLELKGSETTFMCEYADETATIVEFLNRWITFAQSIISTLTGG
GGQGGGGQGGGGQGGGGQGGGGQEVQLLESGGGLVQPGGSLRLSC
AASGRYIDETAVAWFRQAPGKEREFVAGIGGGVDITYYADSVKGR
FTISRDNSKNTLYLQMNSLRPEDTAVYYCAARPGRPLITSKVADL
YPYWGQGTLVTVSSPP.

Here, the VHH fusion of SEQ ID NO:105 is generated essentially as described for Example 1 except that a cDNA sequence encoding SEQ ID NO:105 is used in the expression plasmid.

### Example 7: Recombinant Expression and Purification of VHH-Based Fusion 7

Example 7 is an VHH-IL-2 fusion analog having an amino acid sequence of:

(SEQ ID NO: 106)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQAPQSSSTQQTQLQLEHLLLDLQMILNGIN
NYKNPKLTRMLTFKFYMPKKATELKHLQCLEEELKPLEEVLNLAQ
SKNFHLRPRDLISRINVIVLELKGSETTFMCEYADETATIVEFLN
RWITFAQSIISTLT.

Here, the VHH fusion of SEQ ID NO:106 is generated essentially as described for Example 1 except that a cDNA sequence encoding SEQ ID NO:106 is used in the expression plasmid.

### Example 8: Recombinant Expression and Purification of VHH-Based Fusion 8

Example 8 is a GLP-1-VHH fusion having an amino acid sequence of:

(SEQ ID NO: 107)
HGEGTFTSDVSSYLEEQAAKEFIAWLVKGGGGGGGQGGGGQGGGG
QGGGGQGGGGQEVQLLESGGGLVQPGGSLRLSCAASGRYIDETAV
AWFRQAPGKEREFVAGIGGGVDITYYADSVKGRFTISRDNSKNTL
YLQMNSLRPEDTAVYYCAARPGRPLITSKVADLYPYWGQGTLVTV
SSPP.

Here, the VHH fusion of SEQ ID NO:107 is generated essentially as described for Example 1 except that a cDNA sequence encoding SEQ ID NO:107 is used in the expression plasmid.

### Example 9: Recombinant Expression and Purification of VHH-Based Fusion 9

Example 9 is a Fab-VHH fusion comprising a HC-VHH and a LC, with the HC-VHH amino acid sequence of:

(SEQ ID NO: 108)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTGGGGQGGGGQGGGGQGGGGQGGGGQEVQLLESGGGLVQPGG
SLRLSCAASGRYIDETAVAWFRQAPGKEREFVAGIGGGVDITYYA
DSVKGRFTISRDNSKNTLYLQMNSLRPEDTAVYYCAARPGRPLIT
SKVADLYPYWGQGTLVTVSSPP.
and
the LC amino acid sequence of:
(SEQ ID NO: 109)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

Here, the VHH fusion of SEQ ID NOS:108 and 109 is generated essentially as described for Example 1 except that cDNA sequence encoding SEQ ID NOS: 108 and 109 are cloned into two expression plasmids and then a 1:1 mix of both plasmids is used for the transfection.

### Example 10: Recombinant Expression and Purification of VHH-Based Fusion 10

Example 10 is a Fab-VHH fusion comprising a HC and a LC-VHH, with the HC amino acid sequence of:

(SEQ ID NO: 110)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD.
and
the LC-VHH amino acid sequence of:
(SEQ ID NO: 111)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGQGGGGQG
GGGQGGGGQGGGGQEVQLLESGGGLVQPGGSLRLSCAASGRYIDE
TAVAWFRQAPGKEREFVAGIGGGVDITYYADSVKGRFTISRDNSK
NTLYLQMNSLRPEDTAVYYCAARPGRPLITSKVADLYPYWGQGTL
VTVSSPP.

Here, the VHH fusion analog of SEQ ID NOS:110 and 111 is generated essentially as described for Example 1 except that cDNA sequence encoding SEQ ID NOS:110 and 111 are cloned into two expression plasmids and then a 1:1 mix of both plasmids is used for the transfection.

### Example 11: Recombinant Expression and Purification of VHH-Based Fusion 11

Example 11 is a Fab-VHH fusion comprising a VHH-HC and a LC, with the VHH-HC amino acid sequence of:

(SEQ ID NO: 112)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQEVQLVESGGGLVQPGRSLRLSCAASGFTF
DDYAMHWVRQAPGKGLEWVSAITWNSGHIDYADSVEGRFTISRDN
AKNSLYLQMNSLRAEDTAVYYCAKVSYLSTASSLDYWGQGTLVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCD.
and
the LC amino acid sequence of:
(SEQ ID NO: 113)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

Here, the VHH fusion of SEQ ID NOS:112 and 113 is generated essentially as described for Example 1 except that cDNA sequence encoding SEQ ID NOS:112 and 113 are cloned into two expression plasmids and then a 1:1 mix of both plasmids is used for the transfection.

### Example 12: Recombinant Expression and Purification of VHH-Based Fusion 12

Example 12 is a VHH-Fab comprising a HC and a VHH-LC, with the HC amino acid sequence of:

(SEQ ID NO: 114)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD.
and
the VHH-LC amino acid sequence of:
(SEQ ID NO: 115)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQDIQMTQSPSSLSASVGDRVTITCRASQGI
RNYLAWYQQKPGKAPKLLIYAASTLQSGVPSRFSGSGSGTDFTLT
ISSLQPEDVATYYCQRYNRAPYTFGQGTKVEIKRTVAAPSVFIFP
PSDEQLKSGTASVVCLLNNFYPREAKVQWKVDNALQSGNSQESVT
EQDSKDSTYSLSSTLTLSKADYEKHKVYACEVTHQGLSSPVTKSF
NRGEC

Here, the VHH fusion analog of SEQ ID NOS:114 and 115 is generated essentially as described for Example 1 except that cDNA sequence encoding SEQ ID NOS:114 and 115 are cloned into two expression plasmids and then a 1:1 mix of both plasmids is used for the transfection.

### Example 13: Recombinant Expression and Purification of Control Fab

Example 13 is a Fab comprising a HC and a LC, with the HC amino acid sequence of:

(SEQ ID NO: 120)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD.
and
the LC amino acid sequence of:
(SEQ ID NO: 121)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

Here, the Fab of SEQ ID NOS: 120 and 121 is generated essentially as described for Example 1 except that cDNA sequence encoding SEQ ID NOS: 120 and 121 are cloned into two expression plasmids and then a 1:1 mix of both plasmids is used for the transfection.

### Example 14: Recombinant Expression and Purification of VHH-Based Fusion 13

Example 14 is a GLP-1-VHH fusion having an amino acid sequence of:

(SEQ ID NO: 116)
HGEGTFTSDVSSYLEEQAAKEFIAWLVKGGGGGGGQGGGGQGGGG
QGGGGQGGGGQEVQLLESGGGLVQPGGSLRLSCAASGRYIDETAV
AWFRQAPGKGREFVAGIGGGVDITYYADSVKGRFTISRDNSKNTL
YLQMNSLRPEDTAVYYCAARPGRPLITSKVADLYPYWGQGTLVTV
SSC.

Here, the VHH fusion of SEQ ID NO:116 is generated essentially as described for Example 1 except that a cDNA sequence encoding SEQ ID NO:116 is used in the expression plasmid.

### Example 15: Production of VHH-Based Conjugate 1

Example 15 is a GLP-1/ACTH-VHH conjugate having a chemical structure of: HGEGTFTSDVSSYLEEQAAKEFIAWLVKGGGGGGGQGGGGQGGGGQGGGGQG GGGQEVQLLESGGGLVQPGGSLRLSCAASGRYIDETAVAWFRQAPGKGREFVAG IGGGVDITYYADSVKGRFTISRDNSKNTLYLQMNSLRPEDTAVYYCAARPGRPLI TSKVADLYPYWGQGTLVTVSSC-maleimide-(PEG)₁₂-ACTH (in a C-terminal to N-terminal orientation; SEQ ID NO:117). The VHH conjugate of SEQ ID NO:117 is generated as follows. 1 mg of the VHH fusion of Example 14 (SEQ ID NO: 116) is prepared at 1 mg/mL (˜50 μM) by diluting a 10 mg/mg stock solution 10-fold with PBS pH 7.2. Four equivalents of tris(2-carboxyethyl)phosphine hydrochloride (TCEP) in PBS, pH 7.2, are added and incubated for 4 hours at RT. Successful reduction to homogenous monomers is confirmed via mass spectrometry.

The reaction mixture is desalted using a 2 mL 7K MW Cutoff ZEBA desalting column. Column storage buffer is removed by spinning at 1000 rcf for 1 min. The column is washed by adding 2 mL of PBS pH 7.2 and spinning at 1000 rcf for 1 min. The reaction mixture is loaded onto a ZEBA column and desalted by spinning for 1 min at 1000 rcf into a clean 15 ml conical vial. The desalted sample is concentrated to ˜700 μM (˜100 μL) using a 3K MW cutoff Amicon Ultra spin column.

ACTH-(PEG)₁₂-maleimide-NH₂ (SEQ ID NO:119) is prepared at 20 mg/mL (5.2 mM) in PBS pH 7.2. 20 μL ACTH-(PEG)₁₂-maleimide-NH₂ corresponding to 2 stoichiometric equivalents is added to the desalted and concentrated solution of VHH fusion of Example 14 (SEQ ID NO:116). A white precipitate forms immediately. The reaction is allowed to continue for 10 min., and the solution is solubilized by lowering the pH to 5.5 by slowing adding 0.1 M HCl until solution is clear. Successful conjugation is confirmed via mass spectrometry.

The desired product is purified on an AKTA purification system using a 5 mL MabSelect Protein A resin (GE Healthcare) column with a loading buffer of PBS, pH 7.2, and an elution buffer of 20 mM citrate, pH 3. The sample is loaded onto the column, and loading buffer phase is held until a peak for unreacted ACTH-(PEG)₁₂-maleimide-NH₂ passes through the column. When the signal returns to baseline, the buffer is switched to 20 mM citrate, pH 3, to elute the desired product from the column. 1 mL fractions are collected upon elution, pooled and the pH is adjusted to 7 using tris HCl, pH 8.

To open the maleimide ring, the pH is adjusted to 8 by adding 0.1 M NaOH and left overnight at RT. To drive partial ring opening to completion, the pH is adjusted to 8.5 and incubated for another 24 hr at RT. Completion of ring opening is confirmed by mass spectrometry, and a final pH adjustment back to about 7.2 is performed by adding 0.1 N HCl. The VHH conjugate of Example 15 is then stored at 4° C.

### Example 16: Production of VHH-Based Conjugate 2

Example 16 is an ACTH-VHH conjugate having a chemical structure of: EVQLLESGGGLVQPGGSLRLSCAASGRYIDETAVAWFRQAPGKGREFV AGIGGGVDITYYADSVKGRFTISRDNSKNTLYLQMNSLRPEDTAVYYCAARPGR PLITSKVADLYPYWGQGTLVTVSSC-maleimide-(PEG)₁₂-(ACTH) (in a C-terminal to N-terminal orientation; SEQ ID NO:118).

The VHH conjugate of SEQ ID NO: 118 is generated as follows. 1 mg of a control VHH (SEQ ID NO:37) is prepared at 1 mg/mL (about 70 μM) by diluting a 10 mg/mg stock solution 10-fold with PBS, pH 7.2. Four equivalents of TCEP in PBS, pH 7.2, are added and incubated for 4 hr at RT. Successful reduction to homogenous monomers is confirmed via mass spectrometry.

The reaction mixture is desalted using a 2 mL 7K MW Cutoff ZEBA desalting column. Column storage buffer is removed by spinning at 1000 rcf for 1 min. The column is washed by adding 2 mL of PBS, pH 7.2, and spinning at 1000 rcf for 1 min. The reaction mixture is loaded onto the ZEBA column and desalted by spinning for 1 min at 1000 rcf into a clean 15 ml conical vial. The desalted sample is concentrated to about 700 μM (˜100 μL) using a 3K MW cutoff Amicon Ultra spin column.

ACTH-(PEG)₁₂-maleimide-NH₂ (SEQ ID NO:119) is prepared and conjugated to the VHH as described in Example 15.

### In Vitro Function—Albumin Binding

### Example 17: Albumin-Binding Studies Via SPR of VHH Moieties

In vitro binding of various VHH moieties to human, cynomolgus monkey, mouse, rat, pig, dog and cow serum albumin (SA) is determined by SPR. In particular, the binding of the VHH moieties herein to the SA of these species is summarized below in Table 1. Binding of the VHH moieties of SEQ ID NOS:3 and 8 to 28 to various SAs is carried out on Biacore 8K instrument.

Immobilization of SA to a Series S Sensor Chip CM5 surface is performed according to the manufacturer's instructions (amine coupling kit BR-1000-50). Briefly, carboxyl groups on the sensor chip surfaces (flow cell 1 and 2) are activated by injecting 70 μL of a mixture containing 75 mg/mL 1-ethyl-3-(3-dimethylaminopropyl) carbodiimide hydrochloride (EDC) and 11.5 mg/mL N-hydroxysuccinimide (NHS) at 10 μL/min. Human, cynomolgus monkey, mouse, rat, pig, dog and cow SA are diluted in 10 mM sodium acetate, pH 4.0, (BR-1003-49) at 1, 1, 3, 1, 1, 1 and 1 μg/mL and then injected over the activated chip surfaces (flow cell 2, channel 1 to 8) at 10 μL/min for 90 sec. Human SA is obtained from Sigma Aldrich (St. Louis, MO; Cat. No. A8763). Cynomolgus monkey SA is obtained from Athens R&T (Athens, GA; Cat. No. 16-16-011202-CM). Mouse SA is obtained from Sigma Aldrich (Cat. No. A3559). Rat SA is obtained from Sigma Aldrich (Cat. No. A4538). Pig SA is obtained from Sigma Aldrich (Cat. No. A4414). Dog SA is obtained from Molecular Innovations (Novi, MI; Cat. No. DSA-1213 NC0739153). Cow SA is obtained from Sigma Aldrich (Cat. No. A7030). The various SAs are covalently immobilized through free amines onto a carboxymethyl dextran-coated sensor chip CM5 targeting a surface density of average approximately 77 (58-98) RU. Excess reactive groups on the surfaces (flow cell 1 and 2) are deactivated by injecting 70 μL of 1 M ethanolamine hydrochloride-NaOH, pH 8.5, at 10 μL/min.

VHH moieties are diluted in HBS-EP+buffer (10 mM HEPES, pH 7.6, 150 mM NaCl, 3 mM EDTA, and 0.05% Polysorbate 20) at concentrations of 300 nM. 150 μL of sample is individually injected sequentially across the immobilized SAs surface and is dissociated for 600 sec at a flow rate of 50 L/min at 25 C. The surface is regenerated by injecting 10 mM glycine-HCl, pH 1.5, (BR-1003-54) at 50 μL/min for 100 sec. The resulting sensorgrams are analyzed using Biacore 8K Insight Evaluation Software (version 2.0.15.12933) to calculate the dissociation rate (kd).

TABLE 1
Binding of Exemplary VHH Moieties to Human, Cynomolgus
Monkey, Mouse, Rat, Pig, Dog, and Cow SA at 25° C.

| | VHH Moiety | | | | | | |
|---|---|---|---|---|---|---|---|
| | Human | Cyno | Mouse | Rat | Pig | Dog | Cow |
| | SA | SA | SA | SA | SA | SA | SA |
| SEQ | kd | kd | kd | kd | kd | kd | kd |
| ID NO: | (1/s) | (1/s) | (1/s) | (1/s) | (1/s) | (1/s) | (1/s) |
| 3 | 8.0E−04 | 2.0E−03 | 4.3E−03 | 3.5E−03 | 5.2E−03 | 2.2E−03 | 1.5E−02 |
| 3 | 7.8E−04 | 1.9E−03 | 4.3E−03 | 3.4E−03 | 5.2E−03 | 2.3E−03 | 1.4E−02 |
| 8 | 1.0E−03 | 3.0E−03 | 3.2E−03 | 7.5E−03 | 9.2E−03 | 2.2E−03 | 1.5E−02 |
| 9 | 1.2E−03 | 3.2E−03 | 1.3E−02 | 1.1E−02 | 1.2E−02 | 1.1E−02 | very |
| | | | | | | | weak |
| 10 | weak | very | very | very | very | very | no |
| | | weak | weak | weak | weak | weak | binding |
| 11 | 7.4E−03 | very | no | no | no | no | no |
| | | weak | binding | binding | binding | binding | binding |
| 12 | 1.1E−02 | very | no | no | no | no | no |
| | | weak | binding | binding | binding | binding | binding |
| 13 | very | no | no | no | no | no | no |
| | weak | binding | binding | binding | binding | binding | binding |
| 14 | 1.3E−02 | no | no | no | no | no | no |
| | | binding | binding | binding | binding | binding | binding |
| 15 | 5.8E−03 | very | no | no | no | no | no |
| | | weak | binding | binding | binding | binding | binding |
| 16 | 3.9E−03 | 1.5E−02 | no | no | no | very | no |
| | | | binding | binding | binding | weak | binding |
| 17 | 1.5E−03 | 2.2E−03 | 1.0E−02 | 8.1E−03 | 1.3E−02 | 8.2E−03 | 1.5E−02 |
| 3 | 7.7E−04 | 1.7E−03 | 4.2E−03 | 3.1E−03 | 5.1E−03 | 2.0E−03 | 1.5E−02 |
| 18 | 7.6E−03 | 7.2E−03 | very | 1.4E−02 | very | 1.5E−02 | no |
| | | | weak | | weak | | binding |
| 19 | 4.5E−03 | 7.3E−03 | 1.5E−02 | 1.5E−02 | 1.4E−02 | 1.4E−02 | no |
| | | | | | | | binding |
| 20 | 9.2E−03 | no | no | no | no | very | no |
| | | binding | binding | binding | binding | weak | binding |
| 21 | 2.5E−03 | 1.4E−02 | 1.3E−02 | 1.1E−02 | 1.0E−02 | 8.1E−03 | very |
| | | | | | | | weak |
| 22 | 2.8E−03 | 1.4E−02 | 1.0E−02 | 1.0E−02 | 1.1E−02 | 7.4E−03 | very |
| | | | | | | | weak |
| 23 | no | no | no | no | no | no | no |
| | binding | binding | binding | binding | binding | binding | binding |
| 24 | no | no | no | no | no | no | no |
| | binding | binding | binding | binding | binding | binding | binding |
| 25 | very | no | very | very | very | very | no |
| | weak | binding | weak | weak | weak | weak | binding |
| 26 | very | no | no | no | no | no | no |
| | weak | binding | binding | binding | binding | binding | binding |
| 27 | 1.4E−02 | no | no | no | no | no | no |
| | | binding | binding | binding | binding | binding | binding |
| 3 | 8.6E−04 | 1.5E−03 | 4.2E−03 | 3.0E−03 | 5.0E−03 | 1.9E−03 | 1.5E−02 |
| 4 | 2.1E−04 | 5.3E−04 | 4.3E−03 | 3.3E−03 | 5.7E−03 | 1.7E−03 | n/d |
| 125 | 9.4E−05 | 6.0E−04 | 5.6E−03 | 3.8E−03 | 6.5E−03 | 2.1E−03 | n/d |
| 126 | 1.4E−04 | 7.2E−04 | 5.4E−03 | 5.3E−03 | 5.8E−03 | 2.1E−03 | n/d |

### Example 18: VHH-Based Fusions Albumin-Binding Studies Via SPR

In vitro binding of VHH-based fusions to human, cynomolgus monkey, mouse, rat, pig, dog, cow and rabbit SA is determined by SPR at 25° C. In particular, the affinity of the VHH-fusions of Examples 1 to 9 to SA of these species is summarized below in Tables 2 to 10.

Binding of the VHH-based fusions of Examples 1 to 9 to various SAs is carried out on a Biacore 8K instrument. The immobilization of various SA orthologs to a Series S Sensor Chip CM5 (BR-1006-68) surface are performed according to manufacturer's instructions (amine coupling kit BR-1000-50). Briefly, carboxyl groups on the sensor chip surfaces (flow cell 1 and 2) are activated by injecting 70 μl of mixture containing 75 mg/ml 1-Ethyl-3-(3-dimethylaminopropyl) carbodiimide hydrochloride (EDC), and 11.5 mg/ml N-Hydroxysuccinimide (NHS), at 10 μl/min. Human, cynomolgus monkey, rat, mouse, dog, pig, cow, and rabbit SA are diluted in 10 mM sodium acetate pH 4.0 (BR-1003-49) at 1 and 0.8 μg/ml, 1 and 0.8 μg/ml, 1.5 and 0.8 μg/ml, 4 and 2.5 μg/ml, 1 and 0.8 μg/ml, 1 and 1 μg/ml, 1 and 1 μg/ml, and 1 and 1.5 μg/ml, and then are injected over the activated chip surfaces (flow cell 2, channel 1 to 8) at 10 μl/min for 100 seconds. Human SA is obtained from Sigma Aldrich (Cat. No. A8763). Cynomolgus monkey SA is obtained from Athens R&T (Cat. No. 16-16-011202-CM). Mouse SA is obtained from Sigma Aldrich (Cat. No. A3139). Rat SA is obtained from Sigma Aldrich (Cat. No. A4538). Pig SA is obtained from Sigma Aldrich (Cat. No. A4414). Dog SA is obtained from Molecular Innovations (Novi, MI; Cat. No. DSA-1213 NC0739153). Cow SA is obtained from Sigma Aldrich (Cat. No. A7030). Rabbit SA is obtained from Fitzgerald Industries International (Acton, MA; Cat. No. 30R-3303). The SAs are covalently immobilized through free amines onto a carboxymethyl dextran-coated sensor chip CM5 at surface densities of 25-78 resonance units (RU) for human, cynomolgus monkey, rat, mouse, dog, pig, and cow SA as well as 118-372 resonance units (RU) for rabbit SA. Excess reactive groups on the surfaces (flow cell 1 and 2) are deactivated by injecting 70 μl of 1 M Ethanolamine hydrochloride-NaOH pH 8.5.

VHH-fusions are diluted in HBS-EP+buffer (10 mM HEPES pH 7.6, 150 mM NaCl, 3 mM EDTA, 0.05% Polysorbate 20) at concentrations of 1000, 333.3, 111.1, 37.04, 12.35, 4.12, 1.37, 0.457, 0.152, 0.051 and 0.017 nM. 180 μl of sample are individually injected sequentially across the immobilized SAs on the chip's surface and dissociated for 600 sec at 60 μl/min flow rate at 25° C. The surface is regenerated by injecting 10 mM glycine-HCl pH 1.5 (BR-1003-54) at 60 μl/min for 100 sec. The resulting sensorgrams are analyzed using Biacore 8K Insight Evaluation Software (version 3.0.11.15423) 1:1 binding kinetics model fitting to calculate the binding kinetic parameters: association rate (ka), dissociation rate (kd), and equilibrium dissociation constant (K_{D}).

TABLE 2
Binding Kinetics of VHH-Based Fusion of Example
1 to Human, Cynomolgus Monkey, Mouse, Rat,
Pig, Dog, Cow and Rabbit SA at 25° C.

| Binding to Immobilized SAs | ka (1/Ms) | kd (1/s) | K_{D} (M) |
|---|---|---|---|
| Human SA | 7.6E+04 | 2.4E−04 | 3.2E−09 |
| Cyno SA | 6.8E+04 | 8.6E−04 | 1.3E−08 |
| Mouse SA | 8.1E+04 | 8.2E−03 | 1.0E−07 |
| Rat SA | 7.5E+04 | 6.3E−03 | 8.3E−08 |
| Pig SA | 5.8E+04 | 1.1E−02 | 1.9E−07 |
| Dog SA | 7.9E+04 | 3.2E−03 | 4.0E−08 |
| Cow SA | 9.1E+04 | 7.4E−02 | 8.2E−07 |

| | |
|---|---|
| Rabbit SA | No binding |

K_{D} is determined as 3.2, 13, 100, 83, 190, 40 and 820 nM for human, cynomolgus monkey, mouse, rat, pig, dog and cow SA binding, respectively, with the VHH-based fusion of Example 1.

TABLE 3
Binding Kinetics of VHH-Based Fusion of Example
2 to Human, Cynomolgus Monkey, Mouse, Rat,
Pig, Dog, Cow and Rabbit SA at 25° C.

| Binding to Immobilized SAs | ka (1/Ms) | kd (1/s) | K_{D} (M) |
|---|---|---|---|
| Human SA | 4.3E+05 | 2.5E−04 | 5.8E−10 |
| Cyno SA | 5.0E+05 | 6.0E−04 | 1.2E−09 |
| Mouse SA | 4.8E+05 | 4.2E−03 | 8.8E−09 |
| Rat SA | 4.9E+05 | 3.0E−03 | 6.1E−09 |
| Pig SA | 3.0E+05 | 5.7E−03 | 1.9E−08 |
| Dog SA | 4.6E+05 | 1.7E−03 | 3.7E−09 |
| Cow SA | 4.6E+05 | 5.2E−02 | 1.1E−07 |

| | |
|---|---|
| Rabbit SA | No binding |

K_{D} is determined as 0.58, 1.2, 8.8, 6.1, 19, 3.7 and 110 nM for human, cynomolgus monkey, mouse, rat, pig, dog and cow SA binding, respectively, with the VHH-based fusion of Example 2.

TABLE 4
Binding Kinetics of VHH-Based Fusion of Example
3 to Human, Cynomolgus Monkey, Mouse, Rat,
Pig, Dog, Cow and Rabbit SA at 25° C.

| Binding to Immobilized SAs | ka (1/Ms) | kd (1/s) | K_{D} (M) |
|---|---|---|---|
| Human SA | 1.2E+05 | 2.3E−04 | 1.9E−09 |
| Cyno SA | 1.4E+05 | 8.7E−04 | 6.1E−09 |
| Mouse SA | 1.4E+05 | 8.8E−03 | 6.2E−08 |
| Rat SA | 1.2E+05 | 6.0E−03 | 5.2E−08 |
| Pig SA | 9.3E+04 | 1.2E−02 | 1.3E−07 |
| Dog SA | 1.7E+05 | 3.2E−03 | 1.9E−08 |
| Cow SA | 1.6E+05 | 7.2E−02 | 4.7E−07 |

| | |
|---|---|
| Rabbit SA | No binding |

K_{D} is determined as 1.9, 6.1, 62, 52, 130, 19 and 470 nM for human, cynomolgus monkey, mouse, rat, pig, dog and cow SA binding, respectively, with the VHH-based fusion of Example 3.

TABLE 5
Binding Kinetics of VHH-Based Fusion of Example
4 to Human, Cynomolgus Monkey, Mouse, Rat,
Pig, Dog, Cow and Rabbit SA at 25° C.

| Binding to Immobilized SAs | ka (1/Ms) | kd (1/s) | K_{D} (M) |
|---|---|---|---|
| Human SA | 1.0E+06 | 1.9E−04 | 1.9E−10 |
| Cyno SA | 6.8E+05 | 6.3E−04 | 9.4E−10 |
| Mouse SA | 6.6E+05 | 4.2E−03 | 6.4E−09 |
| Rat SA | 9.7E+05 | 3.0E−03 | 3.1E−09 |
| Pig SA | 4.0E+05 | 6.0E−03 | 1.5E−08 |
| Dog SA | 7.8E+05 | 1.7E−03 | 2.2E−09 |
| Cow SA | 5.7E+05 | 5.8E−02 | 1.0E−07 |

| | |
|---|---|
| Rabbit SA | No binding |

K_{D} is determined as 0.19, 0.94, 6.4, 3.1, 15, 2.2 and 100 nM for human, cynomolgus monkey, mouse, rat, pig, dog and cow SA binding, respectively, with the VHH-based fusion of Example 4.

TABLE 6
Binding Kinetics of VHH-Based Fusion of Example
5 to Human, Cynomolgus Monkey, Mouse, Rat,
Pig, Dog, Cow and Rabbit SA at 25° C.

| Binding to Immobilized SAs | ka (1/Ms) | kd (1/s) | K_{D} (M) |
|---|---|---|---|
| Human SA | 6.7E+05 | 1.4E−04 | 2.0E−10 |
| Cyno SA | 7.4E+05 | 3.4E−04 | 4.5E−10 |
| Mouse SA | 1.4E+06 | 1.2E−03 | 8.6E−10 |
| Rat SA | 1.0E+06 | 7.7E−04 | 7.4E−10 |
| Pig SA | 8.5E+05 | 2.8E−03 | 3.3E−09 |
| Dog SA | 1.1E+06 | 6.8E−04 | 6.0E−10 |
| Cow SA | *not | *not | 2.8E−08 |
| | applicable | applicable | |

| | |
|---|---|
| Rabbit SA | No binding |

K_{D} is determined as 0.2, 0.45, 0.86, 0.74, 3.3, 0.6 and 28 nM for human, cynomolgus monkey, mouse, rat, pig, dog and cow SA binding, respectively, with the VHH-based fusion of Example 5.

TABLE 7
Binding Kinetics of VHH-Based Fusion of Example
6 to Human, Cynomolgus Monkey, Mouse, Rat,
Pig, Dog, Cow and Rabbit SA at 25° C.

| Binding to | | | |
|---|---|---|---|
| Immobilized SAs | ka (1/Ms) | kd (1/s) | K_{D} (M) |
| Human SA | 1.8E+05 | 2.6E−04 | 1.5E−09 |
| Cyno SA | 1.8E+05 | 8.8E−04 | 5.0E−09 |
| Mouse SA | 1.7E+05 | 8.7E−03 | 5.0E−08 |
| Rat SA | 1.9E+05 | 6.6E−03 | 3.4E−08 |
| Pig SA | 1.2E+05 | 1.2E−02 | 1.0E−07 |
| Dog SA | 1.7E+05 | 3.4E−03 | 1.9E−08 |
| Cow SA | 1.9E+05 | 7.1E−02 | 3.8E−07 |

| | |
|---|---|
| Rabbit SA | No binding |

K_{D} is determined as 1.5, 5.0, 50, 34, 100, 19 and 380 nM for human, cynomolgus monkey, mouse, rat, pig, dog and cow SA binding, respectively, with the VHH-based fusion of Example 6.

TABLE 8
Binding Kinetics of VHH-Based Fusion of Example
7 to Human, Cynomolgus Monkey, Mouse, Rat,
Pig, Dog, Cow and Rabbit SA at 25° C.

| Binding to | | | |
|---|---|---|---|
| Immobilized SAs | ka (1/Ms) | kd (1/s) | K_{D} (M) |
| Human SA | 9.1E+05 | 2.1E−04 | 2.3E−10 |
| Cyno SA | 1.4E+06 | 5.6E−04 | 3.9E−10 |
| Mouse SA | 9.7E+05 | 3.8E−03 | 3.9E−09 |
| Rat SA | 9.7E+05 | 2.9E−03 | 3.0E−09 |
| Pig SA | 4.5E+05 | 5.8E−03 | 1.3E−08 |
| Dog SA | 9.2E+05 | 1.6E−03 | 1.8E−09 |
| Cow SA | 6.7E+05 | 5.2E−02 | 7.8E−08 |

| | |
|---|---|
| Rabbit SA | No binding |

K_{D} is determined as 0.23, 0.39, 3.9, 3.0, 13, 1.8 and 78 nM for human, cynomolgus monkey, mouse, rat, pig, dog and cow SA binding, respectively, with the VHH-based fusion of Example 7.

TABLE 9
Binding Kinetics of VHH-Based Fusion of Example
8 to Human, Cynomolgus Monkey, Mouse, Rat,
Pig, Dog, Cow and Rabbit SA at 25° C.

| Binding to | | | |
|---|---|---|---|
| Immobilized SAs | ka (1/Ms) | kd (1/s) | K_{D} (M) |
| Human SA | 7.2E+05 | 1.1E−04 | 1.5E−10 |
| Cyno SA | 9.1E+05 | 8.7E−04 | 9.6E−10 |
| Mouse SA | 5.6E+05 | 1.0E−02 | 1.8E−08 |
| Rat SA | 6.6E+05 | 7.1E−03 | 1.1E−08 |
| Pig SA | 4.9E+05 | 1.3E−02 | 2.7E−08 |
| Dog SA | 7.1E+05 | 3.8E−03 | 5.4E−09 |
| Cow SA | 8.0E+05 | 9.3E−02 | 1.2E−07 |

| | |
|---|---|
| Rabbit SA | No binding |

K_{D} is determined as 0.15, 0.96, 18, 11, 27, 5.4 and 120 nM for human, cynomolgus monkey, mouse, rat, pig, dog and cow SA binding, respectively, with the VHH-based fusion of Example 8.

TABLE 10
Binding Kinetics of VHH-Based Fusion of Example
9 to Human, Cynomolgus Monkey, Mouse, Rat,
Pig, Dog, Cow and Rabbit SA at 25° C.

| Binding to | | | |
|---|---|---|---|
| Immobilized SAs | ka (1/Ms) | kd (1/s) | K_{D} (M) |
| Human SA | 2.1E+05 | 2.8E−04 | 1.3E−09 |
| Cyno SA | 2.2E+05 | 9.4E−04 | 4.3E−09 |
| Mouse SA | 2.3E+05 | 1.0E−02 | 4.3E−08 |
| Rat SA | 2.1E+05 | 7.1E−03 | 3.3E−08 |
| Pig SA | 1.5E+05 | 1.3E−02 | 8.3E−08 |
| Dog SA | 2.4E+05 | 3.7E−03 | 1.5E−08 |
| Cow SA | 2.5E+05 | 8.5E−02 | 3.4E−07 |

| | |
|---|---|
| Rabbit SA | No binding |

K_{D} is determined as 1.3, 4.3, 43, 33, 83, 15, and 340 nM for human, cynomolgus monkey, mouse, rat, pig, dog and cow SA binding, respectively, with the VHH-based fusion of Example 9.

### In Vitro Function-Specific Activity of Payloads

### Example 19: In Vitro Potency of VHH-Based Fusions of Examples 1 and 2

Human CNTFR pSTAT3 assay: IMR-32 cells (ATCC, Cat #CCL-127), which endogenously express human CNTFR, LIFR and gp130 are cultured at 37° C., 5% CO₂, 90% humidity in RPMI-1640 (ATCC, Cat #30-2001) media supplemented with final 10% FBS, 1 mM sodium pyruvate and 1× Antibiotic-Antimycotic (Thermo Fisher Scientific, 15240062). On Day −1 (the day before the pSTAT3 assay), cells are washed once with 1×PBS, lifted from flasks with cell dissociation buffer (Gibco, 13151) and resuspended in above mentioned culture media. Cells are plated in 96-well poly D lysine coated plates (Corning; Cat #354640) at 150,000 cells/0.1 mL/well. Cells are cultured at 37° C., 5% CO₂, 90% humidity overnight. On Day 1 (the day of the pSTAT3 assay), medium is aspirated off and is replaced with 100 μL EMEM (ATCC, Cat #30-2003). Cells are serum starved at 37° C., 5% CO₂, 90% humidity for 4 hr, then 50 μL of 3x serially diluted human CNTF (R&D Systems; Cat #257-NT-010), Example 1 or Example 2 in 0.3% IgG free BSA (Jackson ImmunoResearch Laboratories, Inc.; Cat #001-000-162) are added. EMEM is added for a final 1× concentration. Cells are incubated with these serially diluted proteins for an additional 10 min at 37° C., 5% CO₂, 90% humidity. After the incubation period is complete, contents are removed from the plate. Cells are lysed, and pSTAT3 is detected using an AlphaLISA Surefire Ultra p-STAT3 (Tyr705) Assay Kit (Perkin Elmer; ALSU-PST3) and its two-plate assay protocol for adherent cells. Plates are read on an Envision 2102 instrument (Perkin Elmer).

Statistical analysis of data: Data is imported from an Envision 2102 instrument reader into Excel® (Microsoft). % of maximum (1 nM) human CNTF stimulated pSTAT3 signal is calculated for each concentration of protein tested. EC₅₀ values are generated from these calculations by a variable slope-four parameter dose response curve analysis using GraphPad Prism® software (GraphPad Software, LLC; La Jolla, CA; version 8.4.3). The results of the assay are presented below in Table 11.

TABLE 11
In Vitro Potency of VHH-Based
Fusions to Human CNTF Receptor.

| | hCNTFR EC₅₀ | | |
|---|---|---|---|
| Compound | pM GeoMean | SEM | N |
| human CNTF | 3.50 | 0.45 | 2 |
| Example 1 | 7.09 | 0.35 | 2 |
| Example 2 | 8.39 | 0.65 | 2 |

### Example 20: In Vitro Potency of VHH-Based Fusions of Examples 3 and 4

A cell-based dimerization bioassay from DiscoverX/eurofins (PathHunter eXpress ErbB4/ErbB4 Dimerization Assay Cat #93-0961E3) is performed to test potency of the VHH fusions of Examples 3 and 4 at the human Eb4 receptor (NCBI Ref. Seq. No. NP_001036064.1). The assay detects ligand induced dimerization of two subunits of a receptor-dimer pair and is designed to assess potency. Testing is performed according to the protocol provided by the manufacturer. Briefly, cells are thawed and are plated in the provided 96-well plate at 100 μL per well. After 24 hr incubation at 37° C. in 5% CO₂, 10 μL 11× of Example 3, Example 4 and a control agonist, rhNRG-1 (eurofins cat #92-1091), are added in an 11-point serial dilution curve (1:3) to appropriate wells in duplicate. The plate is incubated at 37° C. for 6 hr. Following the 6-hour incubation, 110 μL detection reagents are added to each well. The plate is incubated at RT in the dark for an additional hr. The chemiluminescent signal is read on plate reader using a 0.5 sec integration time (SpectraMax i3x plate reader by Molecular Devices). Statistical analysis of raw data is performed with GraphPad PRISM version 9.0. The average background reading is subtracted from all values, raw data are transformed and a non-linear regression analysis (log(agonist) vs. response—variable slope (four parameters)) is run to obtain EC₅₀ values and to determine goodness of fit (n=2 for all data).

TABLE 12
Potency of VHH-Based Fusions of Examples 3 and
4 in the DiscoverX PathHunter ErbB4 Bioassay.

| | rhNRG-1 | Example 3 | Example 4 |
|---|---|---|---|
| EC₅₀ (ng/mL) | 16.51 | 33.55 | 112.2 |
| R² | 0.9884 | 0.9863 | 0.9986 |

### Example 21: In Vitro Potency of VHH-Based Fusion of Example 5

Generating HEK293 human GFRAL- and human RET-expressing cell line: HEK293 cells (ATCC) are cultured in DMEM with 10% FBS and 25 mM HEPES, 1x antibiotics and split 1:16 every 3-4 days with TrypLE™ Express (Gibco). Cells are transfected with plasmid DNA of human GFRAL (GDNF Receptor Alpha Like; NCBI Reference Sequence No. NP_997293.2), human RET (Proto-oncogene tyrosine-protein kinase receptor RET; NCBI Reference Sequence No. NP_066124.1), and Fugene 6 (Promega) according to the manufacturer's instructions. Transfected cells are selected with geneticin (Gibco, 1 mg/ml) and puromycin (Gibco, 0.1 mg/ml) for 3-4 weeks. Clonal lines are obtained by limited dilution cloning into 96-well plates and are confirmed with a GDF15 response by AlphaLISA SureFire Ultra p-ERK1/2 (Thr202/Tyr204) Assay Kit (PerkinElmer). Clones are expanded, harvested, resuspended in freezing media, aliquoted into cryovials, and kept in liquid nitrogen for long-term storage. The top responder is selected with the best GDF15 response (signal to background ratio), clonal line #7.

Human GFRAL and RET receptor AlphaLISA SureFire Ultra p-ERK1/2 (Thr202/Tyr204) assay: HEK293 cell lines expressing the human GFRAL and the human RET are cultured with selection medium (DMEM with 10% FBS with 25 mM HEPES, 1x antibiotics, 1 μg/mL puromycin, 1 mg/mL Geneticin). On Day −3 (the day of cell plating), cells are washed once with PBS, lifted from flasks with TrypLE™ Express, and resuspended in plating medium (DMEM with 25 mM HEPES, 1× antibiotics, 10% FBS). Cells are plated in a 96-well plate (Corning Cat #356461) at 20,000 cells/0.1 mL/well. Cells are cultured at 37° C. 5% CO₂ for 72 hr. On Day 1 (the day of assay), medium is removed and replaced with 50 μL serum-free medium (DMEM with 25 mM HEPES, 1× antibiotics). Plates are incubated at 37° C. for 4 hr, then 50 μL of 2× ligand is added (GDF15, final 1×). Plates are incubated for an additional 10 min at 37° C. After the incubation period is complete, medium is removed from plates by decanting and blotting on white utility wipes. Then, 50 μL of 1× AlphaLISA SureFire Ultra Lysis Buffer is added to each well, and the plates are incubated on a plate shaker at 350 rpm at RT for 10 min. Subsequently, according to the manufacturer's instructions (AlphaLISA SureFire Ultra p-ERK (Thr202/Tyr204) Assay Kit, PerkinElmer Cat #ALSU-PERK-A10K) for the 2-Plate/1-Incubation protocol for adherent cells, 10 μL cell lysate, 5 μL Acceptor Mix, and 5 μL Donor Mix are added to an OptiPlate™-384 plate (PerkinElmer cat #6007290). The plate is sealed, wrapped in foil, incubated for 1 min on a plate shaker at 250 rpm at RT, incubated at RT in the dark for 8 hr, and then read on an En Vision 2102 Multilabel Reader with En Vision Manager software (PerkinElmer).

Statistical analysis of data: Data is imported from the EnVision 2102 Multilabel Reader into GraphPad Prism® software (GraphPad Software, LLC; La Jolla, CA; version 8). EC₅₀ values are generated by a variable slope-four parameter dose response curve analysis.

TABLE 13
Stimulation of ERK1/2 Phosphorylation in HEK293 Cells Expressing
Human GFRAL & RET51 by VHH-Based Fusion of Example 5.

| | hGFRAL/RET | | |
|---|---|---|---|
| Compound | EC₅₀ (pM) | SEM | N |
| Human GDF15 | 2.85 | 1.08 | 2 |
| Example 5 | 101 | 19.5 | 2 |

### Example 22: In Vitro Potency of VHH-Based Fusions of Examples 6 and 7

A cell-based dimerization bioassay from DiscoverX, PathHunter IL-2 BioAssay Kit (eurofins/DiscoverX part #93-1003Y3-00091) is used to evaluate the potency of the VHH-based fusions of Examples 6 and 7. The assay detects IL-2 induced dimerization of two subunits of an IL-2 receptor-dimer pair and is designed to assess IL-2 potency. The assay is performed according to the manufacturer's protocol. Briefly, cells are thawed and plated in the provided 96-well plate at 80 μL per well. After 20 hr incubation at 37° C. in 5% CO₂, 20 μL of a 5× stock concentration of Examples 6 and 7 and the provided IL-2 reference standard is added in an 11-point serial dilution curve (1:3) to appropriate wells in duplicate. The plate is incubated at 37° C. for 6 hr. Following the 6-hr incubation with the Examples 6 and 7 and the reference standard, 10 μL detection reagent 1 are added to each well. Mixing is achieved on a plate shaker at 350 rpm for 1 min, and the plate is incubated at RT in the dark for 15 min. Then 40 μL of detection reagent 2 are added to each well and incubated at RT in the dark for 1 additional hr. Chemiluminescent signal is read on plate reader using a 1-sec integration time (SpectraMax i3× plate reader by Molecular Devices). Statistical analysis of raw data is performed with GraphPad PRISM version 8.4.3. Raw data are transformed and a non-linear regression analysis (log(agonist) vs. response—variable slope (four parameters)) is run to obtain EC₅₀ values and to determine goodness of fit. (n=2 for all data).

TABLE 14
Potency of VHH-Based Fusions of Examples 6 and
7 in the DiscoverX PathHunter IL-2 BioAssay.

| | EC₅₀ (nM) | R² | N |
|---|---|---|---|
| Human IL-2 | 0.13 | 0.9983 | 2 |
| Example 6 | 6.7 | 0.9978 | 2 |
| Example 7 | 0.49 | 0.9980 | 2 |

### Example 23: In Vitro Potency of VHH-Based Fusions of Examples 9 and 13

A L929 cell-based cytotoxicity assay is used to assess in vitro neutralization potency of Examples 9 and 13 of soluble human TNFα, soluble cyno TNFα, or membrane-bound human TNFα.

L929 cells (ATCC) endogenously expressing the human TNFα receptor are cultured in DMEM High Glucose media with 10% heat-inactivated FBS, 2 mM L-glutamine, 1× non-essential amino acids, 1× sodium pyruvate, 1× antibiotics and split 1:20 every 3-4 days with TrypLE™ Express (Gibco).

Soluble human TNFα protein is obtained from custom synthesis by Syngene (Bangalore, India). Soluble cyno TNFα protein is obtained from R&D Systems (Minneapolis, MN; Cat. #1070-RM/CF).

A stable, MT104 H2 CHO-membrane human TNFα-expressing cell line is generated at Eli Lilly and Company (Indianapolis, IN). The cells are cultured in Lilly media LM7300 with 8 mM L-glutamine and selection agent G418 (500 μg/mL) split 1:10 every 3-4 days.

L929 cytotoxicity assay: On Day 1, L929 cells are trypsinized with 5 mL TrypLE™ (Gibco #12605036), complete media is added (1:3 volume) and centrifuged at 1000 rpm for 5 min at RT. The supernatant is gently aspirated, and the cells are re-suspended in 15 mL of complete media (DMEM High Glucose Media with 10% heat-inactivated FBS, 2 mM L-glutamine, 1× non-essential amino acids, 1× sodium pyruvate, 1× antibiotics), and an aliquot of cells is counted using a Viacell counter. Cells are plated in 96-well Poly-D Lysine-coated plates (Corning #354496) at 10,000 cells/0.1 mL/well and cultured at 37° C. 5% CO₂ overnight. On Day 2, Examples 9 and 13 are titrated with fixed amounts of antigens: soluble human TNFα (200 pg/mL) or soluble cyno TNFα (750 pg/mL) or membrane human TNFα expressing cells (5000 cells/mL) in DMEM+FBS with Actinomycin D (6.25 μg/mL). Starting concentrations are 1.5 μg/mL, 3 μg/mL and 10 μg/mL for soluble human TNF, soluble cyno TNF and membrane human TNF neutralization, respectively and are titrated by 3-fold serial dilution 8 points down. Titrated compound-antigen complex is added to 96-well plates containing L929 cells after removing media, and the plates are incubated at 37° C. 5% CO₂ overnight. Stimulated negative control wells contain L929 cells+Actinomycin D+TNFα antigen, whereas unstimulated control wells include only L929 cells+Actinomycin D.

On Day 3, media is removed from 96-well plates, and 120 μL of Cell Titer AQueous ONE substrate solution (1:6 dilution in DMEM media+FBS) is added to each well on all the plates. Plates are read at an OD 490 nm after 2 hr on a Spectra max plate reader using Softmax 4.7 software.

Data Analysis: OD readings versus concentration is graphed in Excel® (Microsoft; Redmond, WA); EC₅₀ calculation and statistical analysis are done using Eli Lilly statistical software tools (Global Stats Discovery Team, Lilly).

TABLE 15
In Vitro Potency of VHH-Based Fusions of Examples
9 and 13 Neutralizing Soluble Human TNFα.

| | EC₅₀ | | | | |
|---|---|---|---|---|---|
| | Geo Mean | | | Log | |
| Compound | (μg/mL) | Error | N | Mean | Sd |
| Example 9 | 0.0562 | 0.0115 | 3 | −1.2506 | 0.0153 |
| Example 13 | 0.0333 | 0.0004 | 3 | −1.4781 | 0.0089 |
| Adalimumab | 0.0392 | 0.0009 | 3 | −1.407 | 0.01678 |

TABLE 16
In Vitro Potency of VHH-Based Fusions of Examples
9 and 13 Neutralizing Soluble Cyno TNFα.

| | EC₅₀ | | | | |
|---|---|---|---|---|---|
| | Geo Mean | | | Log | |
| Compound | (μg/mL) | Error | N | Mean | Sd |
| Example 9 | 0.1748 | 0.0040 | 3 | −0.7574 | 0.0170 |
| Example 13 | 0.1025 | 0.0045 | 3 | −0.9893 | 0.0333 |
| Adalimumab | 0.0944 | 0.0021 | 3 | −0.01633 | 0.0163 |

TABLE 17
In Vitro Potency of VHH-Based Fusions of Examples
9 and 13 Neutralizing Membrane Human TNFα.

| | EC₅₀ | | | | |
|---|---|---|---|---|---|
| | Geo Mean | | | Log | |
| Compound | (μg/mL) | Error | N | Mean | Sd |
| Example 9 | 0.2943 | 0.0040 | 3 | −0.5312 | 0.0102 |
| Example 13 | 0.2005 | 0.0196 | 3 | −0.6979 | 0.0736 |
| Adalimumab | 0.2165 | 0.0068 | 3 | −0.6645 | 0.0238 |

TABLE 18
In Vitro Potency of VHH-Based Fusions of Examples 9 and
13 Neutralizing Various Forms of Human and Cyno TNFα.

EC₅₀ (nM)-Geo Mean

| | Soluble | Soluble | Membrane |
|---|---|---|---|
| | Human TNFα | Cyno TNFα | Human TNFα |
| Compound | Inhibition | Inhibition | Inhibition |
| Example 9 | 0.886 | 2.7565 | 4.641 |
| Example 13 | 0.703 | 2.1668 | 4.238 |
| Adalimumab | 0.2613 | 0.6292 | 1.443 |

### Example 24: In Vitro Activity of Multi-Specific VHH-Based Fusion and Conjugate of Examples 15 and 16

Cell-based cyclic AMP assay kits purchased from DiscoverX (eurofins) are used to evaluate activity of the VHH-based conjugates of Examples 15 and 16 at GLP1R and MC3R. For the CAMP, Hunter eXpress GLP1R CHO-K1 GPCR assay (cat #95-0062E2) is used, and an internal GLP-1 standard (SEQ ID NO:99) is used as a positive control. For the CAMP Hunter eXpress MC3R CHO-K1 GPCR assay (cat #95-0045E2) is used, and α-MSH is used a positive control. Assays are performed according to the GPCR assay protocol provided by the manufacturer. Briefly, cells are thawed and are plated in a 96-well plate at 100 μL per well. After a 24-hr incubation at 37° C. in 5% CO₂, 15 μL 3× agonist (VHH conjugate/fusion and controls) and cAMP standard are added to appropriate wells in duplicate. The plate is incubated at 37° C. for 30 minutes. cAMP working detection solution is made up and kept at RT protected from light. Following a 30-min incubation with a VHH fusion or conjugate, 15 μL CAMP antibody reagent is added to each well. Immediately after adding antibody reagent, 60 μL of a cAMP working detection solution is added, which is made up during the incubation period. The plate is incubated at RT in the dark for 1 hr, then 60 μL of CAMP solution A are added to each well, and the plate is incubated at RT in the dark for 3-18 hr. Chemiluminescent signal is read on a plate reader (SpectraMax i3× plate reader by Molecular Devices). Statistical analysis of raw data is performed with GraphPad PRISM version 8. Raw data is transformed, and a non-linear regression analysis is run (log(agonist) vs. response-variable slope (4 parameters)) to obtain EC₅₀ values and to determine goodness of fit (n=2 for all data).

TABLE 19
In Vitro Potency of VHH-Based Conjugates
of Examples 15 and 16 at MC3R.

| | | | cAMP |
|---|---|---|---|
| | Example 15 | Example 16 | standard |
| EC₅₀ | 54.2 | 46.17 | 36.25 |
| R squared | 0.9941 | 0.9888 | 0.9932 |

TABLE 20
In Vitro Potency of VHH-Based Conjugate
of Example 15 at GLP1R.

| | | GLP-1 | |
|---|---|---|---|
| | | positive | cAMP |
| | Example 15 | control | standard |
| EC₅₀ | 1.807 | 0.5044 | 39.89 |
| R squared | 0.9925 | 0.9832 | 0.9971 |

### In Vivo Function—Long-Acting Specific PD for Each Payload

### Example 25: In Vivo Activity (Body Weight Loss in Healthy Mice) of VHH-Based Fusions of Examples 1 and 2

The study is performed with 48 male DIO C57BL6 mice sourced from Taconic. Mice are 15 weeks of age at study start. Animals are single housed in micro-isolator cages with ad-libitum access to automated house water (2-5 ppm chlorine) and high-fat chow (TD95217). Mice are randomized into 8 groups (n=6) based on body weight using the block randomized allocation tool developed by Lilly statisticians for in vivo group randomization. To reduce potential stress-related study-effects, all animals are acclimated to daily handling for at least 3 days prior to the start of the study. Body weights and food weights are recorded for 2 days prior to study start, then daily throughout the study. Treatments are administered subcutaneously in the interscapular region over the course of the study (QD dosing schedule) using a 0.5 mL 28G insulin syringe (BD cat #329461). Native CNTF (R&D systems, recombinant human CNTF cat #257-NT/CF, lot #GL402101A), Example 1, and Example 2 are diluted to appropriate concentrations in sterile PBS pH 7.2 within 30 minutes of administration. Native CNTF is dosed at 0.25 mpk. Example 1 and Example 2 are dosed at either 0.25 mpk or 0.1 mpk. Dilutions are calculated for each treatment daily based on average group body weights from the previous day. 200 μL of PBS are administer to animals in the vehicle control group. Body weight changes are analyzed using an internal statistical tool with a rigorous model-based approach developed by Lilly statisticians for in vivo PD studies. Animals are removed from study regardless of dosing date upon reaching 20% body weight loss.

TABLE 21
Effect of Varied Doses of VHH-based Fusions of Examples 1
and 2 on Body Weight in Healthy Mice at Various Timepoints.

| | | | % change in body weight by study day | | | | | | |
|---|---|---|---|---|---|---|---|---|---|
| Compound | Dose | N | 0 | 1 | 2 | 3 | 4 | 5 | 6 |
| Vehicle | n/a | 6 | 102.47 | 102.11 | 101.01 | 99.98 | 99.50 | 98.61 | 99.32 |
| Example 1 | 0.1 mpk | 6 | 101.09 | 100.51 | 97.61 | 95.38 | 94.24 | 92.73 | 91.44 |
| Example 1 | 0.25 mpk | 6 | 100.81 | 97.18 | 91.21 | 85.31 | 81.18 | 79.19 | 82.31 |
| Example 2 | 0.1 mpk | 6 | 102.13 | 100.70 | 98.04 | 95.39 | 94.87 | 93.02 | 91.91 |
| Example 2 | 0.25 mpk | 6 | 102.77 | 99.81 | 95.46 | 90.42 | 85.29 | 82.23 | 82.91 |
| rhCNTF | 0.25 mpk | 6 | 100.51 | 98.95 | 96.96 | 94.76 | 94.03 | 91.74 | 90.62 |

TABLE 22
Statistical Analysis of Effect of Varied Doses of VHH-based Fusions of
Examples 1 and 2 on Body Weight in Healthy Mice at Various Timepoints.

| | | | Study day | | | | | | |
|---|---|---|---|---|---|---|---|---|---|
| Compound | Dose | | 0 | 1 | 2 | 3 | 4 | 5 | 6 |
| Example | 0.1 | effect | 0.580 | 1.566 | 0.650 | 0.626 | 0.208 | 0.989 | 0.823 |
| 1 | mpk | size | | | | | | | |
| | | p-val | 0.997 | 0.751 | 0.995 | 0.996 | 1.000 | 0.958 | 0.983 |
| Example | 0.25 | effect | 0.292 | −1.764 | −5.750 | −9.442 | −12.853 | −12.552 | −8.309 |
| 1 | mpk | size | | | | | | | |
| | | p-val | 1.000 | 0.650 | 0.00026 | <0.0001 | <0.0001 | <0.0001 | <0.0001 |
| Example | 0.1 | effect | 1.617 | 1.754 | 1.077 | 0.638 | 0.837 | 1.281 | 1.289 |
| 2 | mpk | size | | | | | | | |
| | | p-val | 0.726 | 0.655 | 0.939 | 0.995 | 0.981 | 0.876 | 0.873 |
| Example | 0.25 | effect | 2.252 | 0.867 | −1.501 | −4.332 | −8.743 | −9.516 | −7.715 |
| 2 | mpk | size | | | | | | | |
| | | p-val | 0.403 | 0.977 | 0.783 | 0.012 | <0.0001 | <0.0001 | <0.0001 |

P-values are in comparison to the native CNTF 0.25 mpk treatment group

As shown in Table 21 and 22, all treatment groups demonstrate significant body weight loss when compared to the vehicle group by study day 3. Example 1 and Example 2 dosing at 0.1 mpk both show body weight loss equivalent to rhCNTF dosed at 0.25 mpk. Animals receiving Example 1 at 0.25 mpk display significantly more body weight loss by study day 2 when compared to rhCNTF at 0.25 mpk. Animals receiving Example 1 at 0.25 mpk display significantly more body weight loss by study day 3 when compared to rhCNTF at 0.25 mpk. Animals in the groups receiving 0.25 mpk dosage of both Example 1 and 2 are not dosed on days 5 or 6 due to clinical signs of dehydration and/or achievement of 20% BWL.

### Example 26: In Vivo Activity (Blood Chemistry Analysis of Healthy Mice) of VHH-Based Fusions of Examples 3 and 4

The study is performed with 48 female C57BL6 mice sourced from Envigo at 6 weeks of age. Animals are pair housed in micro-isolator cages with ad-libitum access to automated house water (2-5 ppm chlorine) and standard chow (Teklad 2014 rodent maintenance diet). Mice are randomized into 6 groups (n=8) based on body weight using the block randomized allocation tool developed by Lilly statisticians for in vivo group randomization. To reduce potential stress-related study-effects, all animals are acclimated to daily handling for at least 3 days prior to study start. Body weights and food weights are recorded for 2 days prior to study start, then monitored and recorded daily throughout the study. Animals are 8 weeks of age at the time of the first dose. Treatments are administered subcutaneously in the interscapular region every other day for 7 days for a total of 3 doses (Q2D dosing schedule) using a 0.5 mL 28G insulin syringe (BD cat #329461). Native NRG-1 (R&D systems, recombinant human NRG1-beta 1 EGF Domain, CF, cat #396-HB/CF, batch #ACD182101A), Example 3 and Example 4 are diluted to appropriate concentrations in sterile PBS pH 7.2 within 30 minutes of administration. Native NRG-1 is dosed at 1 mpk. Example 3 and Example 4 are dosed at 1 and 0.3 mpk. Dilutions for each treatment are calculated daily based on average group body weights from the previous day. Animals in the vehicle control group receive 200 μL of PBS SC. Treatments are administered in the morning on study days 1, 3, & 5. All animals are anesthetized with isofluorane (4%) to a surgical plane and exsanguinated via retro-orbital bleed in accordance with animal care and use protocols on study day 7. Blood is collected into serum separator tubes (BD cat #365967) and kept at room temperature for up to 90 minutes. Blood is spun down for 10 minutes at 10,000 RPM. Serum is collected from each tube and aliquoted for Chem 18 analysis. Serum is analyzed on the Roche Cobas 8000 Modular chemistry analyzer. All reagents for the Chem 18 analysis are sourced from Roche except for the triglyceride test which uses Fujifilm WAKO reagent. JMP software oneway analysis of CI by group using Dunnett's Method is used for statistical analysis of blood chemistry values.

TABLE 23
Statistical Analysis of Effect of Varied Doses of VHH-based Fusions
of Examples 3 and 4 on Blood Chemistry Values of Healthy Mice.

| | | | | | BUN | creatinine | cholesterol | ALP |
|---|---|---|---|---|---|---|---|---|
| Compound | Dose | | N | | mg/dL | mg/dL | mg/dL | IU/L |
| Vehicle | n/a | | 8 | mean | 17.51 | 0.1025 | 79.125 | 173 |
| | | | | p val | 1 | 1 | 1 | 1 |
| Example 3 | 1 | mpk | 8 | mean | 16.63 | 0.08 | 98.375 | 128.75 |
| | | | | p val | 0.99 | 0.0462 | 0.0004* | <0.0001* |
| Example 3 | 0.3 | mpk | 8 | mean | 17.22 | 0.08 | 85 | 156.625 |
| | | | | p val | 0.8 | 0.0462 | 0.5397 | 0.1347 |
| Example 4 | 1 | mpk | 8 | mean | 17.39 | 0.0675 | 173 | 81.25 |
| | | | | p val | 1 | 0.0008* | <0.0001* | <0.0001* |
| Example 4 | 0.3 | mpk | 8 | mean | 14.7 | 0.0787 | 99.75 | 138.25 |
| | | | | p val | 0.015* | 0.0324* | 0.0001* | 0.0002* |
| rhNRG-1 | 1 | mpk | 8 | mean | 14.55 | 0.0775 | 79.375 | 157.5 |
| (native | | | | p val | 0.01* | 0.0224* | 1 | 0.1695 |
| control) | | | | | | | | |

*Indicative of statistically significant difference from vehicle treatment group

As shown in Table 23, the VHH fusions of Examples 3 & 4 demonstrate significant effects on blood chemistry as compared to the vehicle control. A reduction in serum creatinine is observed across all treatment groups with both dose levels of Example 4 and the native rhNRG1 control producing statistically significant drops. A significant decrease in ALP from baseline occurs for animals dosed with the native rhNRG1 control and 1 mpk dosing groups for both Example 3 and 4. For both dose levels of Example 3, at the 1 mpk dose of Example 4, and in the native rhNRG1 control group cholesterol levels are increased. The native rhNRG1 control and the 0.3 mpk dose group of Example 4 also significantly decrease BUN.

### Example 27: In Vivo Activity (Body Weight Loss in Healthy Mice) of VHH-Based Fusion of Example 5

24 male DIO C57BL6 mice are sourced from Taconic at 12 weeks of age. Animals are single housed in micro-isolator cages with ad-libitum access to automated house water (2-5 ppm chlorine) and high-fat chow (TD95217). Randomize mice into 4 groups (n=6) based on body weight using the block randomized allocation tool developed by Lilly statisticians for in vivo group randomization. To reduce potential stress-related study-effects, all animals are acclimated to daily handling for at least 3 days prior to the start of the study. Body weights and food weights are recorded for 2 days prior to study start, then monitored and recorded daily throughout the study. 5 doses of each treatment are administered subcutaneously in the interscapular region over the course of the study (Q3D dosing schedule) using a 0.5 mL 28G insulin syringe (BD cat #329461). Native GDF-15 (R&D systems, recombinant, cat #957-GD/CF; batch EHF232101A) and VHH fusion protein of Example 5 are diluted to appropriate concentrations in sterile PBS pH 7.2 within 30 minutes of administration. Native GDF-15 is dosed at 0.1 mpk and VHH fusion protein of Example 5 is dosed at either 1 mpk or 0.1 mpk based on average group body weights from the previous day. Animals in the vehicle control group receive 200 μL of PBS. Body weight changes are analyzed using an internal statistical tool with a rigorous model-based approach developed by Lilly statisticians for in vivo PD studies.

TABLE 24
Effect of Varied Doses of VHH-based Fusion of Example
5 on Body Weight in Healthy Mice at Various Timepoints.

| | | | % change in body weight by study day | | | | |
|---|---|---|---|---|---|---|---|
| Compound | Dose N | | 0 | 3 | 6 | 9 | 12 |
| Vehicle | n/a | 6 | 100.3 | 101.9 | 100.2 | 102.3 | 104.4 |
| Example 5 | 0.1 | 6 | 100.9 | 95.8 | 90.7 | 89.2 | 86.2 |
| Example 5 | 1 | 6 | 101.9 | 95.6 | 91.3 | 87.7 | 85.5 |
| Native GDF-15 | 0.1 | 6 | 99.7 | 99.2 | 97.1 | 95.7 | 94.8 |

TABLE 25
Statistical Analysis of Effect of Varied Doses of VHH-based Fusion
of Example 5 on Body Weight in Healthy Mice at Various Timepoints.

| | | | Study Day | | | | |
|---|---|---|---|---|---|---|---|
| Compound | Dose | | 0 | 3 | 6 | 9 | 12 |
| Example 5 | 0.1 | Effect size | 0.583 | −6.02 | −9.48 | −13.08 | −18.15 |
| | | P-val | 0.971 | 6.88E−04 | <0.001 | <0.001 | <0.001 |
| Example 5 | 1 | Effect size | 1.657 | −6.247 | −8.862 | −14.644 | −18.825 |
| | | P-val | 0.616 | 4.53E−04 | <0.001 | <0.001 | <0.001 |
| Native | 0.1 | Effect size | −0.631 | −2.673 | −3.030 | −6.611 | −9.589 |
| GDF-15 | | P-val | 0.963 | 0.242 | 0.158 | 1.85E−04 | <0.001 |

P-values are in comparison to the vehicle treated control group

As shown in Tables 24 and 25, significant body weight loss occurs in both groups treated with VHH fusion protein of Example 5 at either 0.1 mpk or 1 mpk by day 3 compared to vehicle control. Body weight loss in the native GDF-15 treated group reaches significance by day 9. There is no difference in body weight loss at any time point between the 1 and 0.1 mpk dose groups of VHH fusion protein of Example 5. Animals receiving 1 mpk or 0.1 mpk VHH fusion protein of Example 5 lost significantly more body weight by day 6 on study as compared to those treated with native GDF15. This significance persists for the remainder of the study.

### Example 28: In Vivo Activity (Corticosterone Induction and Body Weight Loss in Healthy Mice) of VHH-Based Conjugate of Example 15

Healthy 6-week-old female C57BL6 mice are purchased from Envigo, group housed (3 mice per cage) in micro-isolator cages with house filtered water in bottles and fed a standard chow diet (2014 Teklad global rodent maintenance diet). Mice are allowed to acclimate after receipt for at least 72 hr. Mice are weighed 1 day prior to dosing. A single subcutaneous dose of the VHH fusion conjugate of Example 15 at 0.1, 0.3, 1 or 3 nmol/kg, or vehicle (DPBS) is administered on day 1; n=3 per group. A tail stick for collection of dried blood spots (DBS) samples for corticosterone level analysis is performed just prior to dosing (TO) and at 2, 6, 24, 30, 48 and 72 hr post dose. About 20-30 μL of blood from the tail of each animal is collected on Whatman DMPK cards (WB129243). DBS cards are provided to in-house LC-MS specialists for corticosterone level analysis. Animal body weight is also recorded on a daily basis throughout the study. All in vivo experimental procedures are conducted in compliance with IACUC standards and according to an approved animal use protocol (19-033). Data are reported in Tables 26 and 27 as mean corticosterone levels (ng/mL) or mean percent change from body weight recorded on day 0, respectively.

TABLE 26
Effect of Varied Doses of VHH-Based Conjugate of Example 15 on
Corticosterone Levels in Health Mice at Various Timepoints.

| | | | Hours Post Dose: | | | | | | |
|---|---|---|---|---|---|---|---|---|---|
| Dose | N | | 0 | 2 | 6 | 24 | 30 | 48 | 72 |
| 0.1 | 3 | Mean | 8.53 | 331.44 | 542.62 | 52.60 | 32.77 | 16.36 | 7.54 |
| nmol/kg | | SEM | 0.65 | 48.23 | 43.88 | 33.65 | 1.43 | 4.00 | 2.73 |
| | | P-val | 0.17 | 0.018 | 0.007 | 0.27 | 0.032 | 0.2 | 0.002 |
| 0.3 | 3 | Mean | 44.71 | 788.87 | 1278.7 | 132.09 | 53.99 | 5.86 | 18.96 |
| nmol/kg | | SEM | 11.96 | 125.20 | 120.20 | 96.47 | 11.99 | 0.18 | 1.83 |
| | | P-val | 0.49 | 0.006 | 0.0013 | 0.974 | 0.053 | 0.078 | 0.015 |
| 1 | 3 | Mean | 25.16 | 1027.8 | 1315.4 | 1003.8 | 275.68 | 8.02 | 8.28 |
| nmol/kg | | SEM | 15.44 | 52.55 | 109.56 | 297.4 | 151.60 | 3.08 | 0.16 |
| | | P-val | 0.3 | <.0001 | 0.0008 | 0.045 | 0.63 | 0.098 | 0.0006 |
| 3 | 3 | Mean | 52.85 | 1071.9 | 1315.5 | 1746.4 | 1748.0 | 260.69 | 10.24 |
| nmol/kg | | SEM | 19.44 | 63.01 | 15.20 | 82.73 | 92.80 | 187.38 | 2.95 |
| | | P-val | 0.62 | 0.0001 | <.0001 | <.0001 | 0.0001 | 0.302 | 0.005 |
| Vehicle | 3 | Mean | 77.11 | 126.99 | 321.41 | 135.83 | 192.73 | 38.37 | 30.81 |
| | | SEM | 41.11 | 21.57 | 2.44 | 55.38 | 49.68 | 13.82 | 2.30 |

P-values are calculated to determine significance using an unpaired T-test of treatment groups vs Vehicle by individual time point in GraphPad PRISM software.

As shown in Table 26, treatment with VHH-based conjugate of Example 15 leads to significant induction of corticosterone levels as compared to vehicle control at 2 and 6 hours post dose for all dose levels. For animals in the 3 mpk dosing groups, the increase in corticosterone levels remains significantly higher than the vehicle control group out to 30 hours post dose.

TABLE 27
Effect of Varied Doses of VHH-Based Conjugate of Example
15 on Body Weight in Healthy Mice 72 hr Post-Injection.

| Treatment | Dose | | % BW | | |
|---|---|---|---|---|---|
| Group | (nmol/kg) | N | Mean | SEM | P-value |
| Vehicle | n/a | 3 | 106.7 | 1.125 | 1 |
| Example 15 | 0.1 | 3 | 105.1 | 0.563 | 0.2846 |
| Example 15 | 0.3 | 3 | 102.1 | 2.183 | 0.1346 |
| Example 15 | 1 | 3 | 97.9 | 0.671 | 0.0026 |
| Example 15 | 3 | 3 | 100.1 | 1.385 | 0.0211 |

As shown in Table 27, treatment with VHH-based conjugate of Example 15 leads to prevention of body weight gain in the 1 nmol/kg and 3 nmol/kg treatment groups over the course of the study. The difference in percent body weight gain is significant for these two treatment groups by day 3 when compared to vehicle control. P-values are calculated using an unpaired T-test of treatment groups vs Vehicle in GraphPad PRISM software.

### In Vivo Function—PK

### Example 29: PK of VHH-Based Fusions of Example 4 in Male Sprague Dawley Rats

Male Sprague Dawley rats are administered a single intravenous (IV) or subcutaneous (SC) dose of 106.8 nmol/kg for Example 4, formulated in PBS buffer (pH 7.4) at a dose volume of 2.6 mL/kg. Blood is collected for PK characterization at 1, 6, 12, 24, 48, 96, 144, 168 and 240 hr post-dose for IV the route; and at 6, 12, 24, 48, 96, 144, 168 and 240 hr post-dose for the SC route.

Plasma concentrations of the VHH-based fusions of Example 4 are determined by a qualified LC/MS assay at Altascience Company (Laval, Quebec, Canada) using a Q/Exactive Plus mass spectrometer (Thermo Scientific, San Jose, CA). The VHH fusions of Example 4 and an internal standard are isolated from K3EDTA rat plasma via immunoprecipitation with an anti-VHH-antibody-biotin conjugate and streptavidin-coated magnetic beads. Following wash steps to remove interfering endogenous proteins, the isolated VHH fusions of Example 4 are reduced, alkylated and digested with trypsin, and the tryptic peptides are analyzed by LC/MS as a surrogate measure of the intact fusions. The plasma concentrations of the VHH fusions of Example 4 are used to calculate the PK parameters shown in Table 28.

TABLE 28
Mean Plasma Pharmacokinetic Parameters for VHH-Based Fusions of Example 4
Following Single IV or SC Dose Administered to Male Sprague Dawley Rats.

| | | | | C₀ (IV) | | CL (IV) | |
|---|---|---|---|---|---|---|---|
| | | Dose | Tₘₐₓ | Cₘₐₓ (SC) | AUC_{0-inf} | CL/F (SC) | t½ |
| Compound | Route | (nmol/kg) | (hr) | (nmol/L) | (hr*nmol/L) | (mL/hr/kg) | (hr) |
| Example 4 | IV | 106.8 | NA | 2310 | 4.40E4 | 2.45 | 30 |
| | | | | (461) | (5.27E3) | (0.274) | (1.3) |
| | SC | 106.8 | 24 | 127 | 6.83E3 | 16.2 | 38 |
| | | | (0) | (27) | (1.51E3) | (3.50) | (3.2) |

NOTE:
Abbreviations: t½ = half-life, Tₘₐₓ = time to maximum concentration, Cₘₐₓ = maximum observed plasma concentration, C₀ = plasma concentration extrapolated to time zero, AUC_{0-inf} = area under the curve from time 0 hours to infinity, CL = clearance, CL/F = clearance/bioavailability.
Standard deviation values are included in the table below the mean PK parameters, in parentheses.
(N = 3/group)

As shown in Table 28, the VHH fusion of Example 4 demonstrates an extended PK profile in Sprague Dawley rats relative to the non-fused proteins (data not shown).

### Example 30: PK of VHH-Based Fusions of Examples 5, 6 and 7 in Male Sprague Dawley Rats

Male Sprague Dawley rats are administered a single intravenous (IV) or subcutaneous (SQ) dose of 50 nmol/kg for Example 5, 25 nmol/kg for Example 6 or 7, formulated in PBS buffer (pH 7.4) at a dose volume of 4 mL/kg. Blood is collected for PK characterization at 1, 6, 12, 24, 48, 96, 144, 168 and 240 hr post-dose for IV routes; and at 6, 12, 24, 48, 96, 144, 168 and 240 hr post-dose for SQ routes.

Plasma concentrations of the VHH-based fusions of Examples 5-7 are determined by a qualified LC/MS assay at Covance Laboratories (Greenfield, IN) using a Q/Exactive Plus mass spectrometer (Thermo Scientific, San Jose, CA). The VHH fusions of Examples 5-7 and an internal standard are isolated from K3EDTA rat plasma via immunoprecipitation with an anti-VHH-antibody-biotin conjugate and streptavidin-coated magnetic beads. Following wash steps to remove interfering endogenous proteins, the isolated VHH fusions of Examples 5-7 are reduced, alkylated and digested with trypsin, and the tryptic peptides are analyzed by LC/MS as a surrogate measure of the intact fusions. The plasma concentrations of the VHH fusions of Examples 5-7 are used to calculate the PK parameters shown in Table 29.

TABLE 29
Mean Plasma Pharmacokinetic Parameters for VHH-Based Fusions of Examples 5, 6
and 7 Following Single IV or SQ Dose Administered to Male Sprague Dawley Rats.

| | | | | C₀ (IV) | | | |
|---|---|---|---|---|---|---|---|
| | | | | Cₘₐₓ | | CL (IV) | |
| | | Dose | Tₘₐₓ | (SQ) | AUC_{0-inf} | CL/F (SQ) | t½ |
| Compound | Route | (nmol/kg) | (hr) | (nmol/L) | (hr*nmol/L) | (mL/hr/kg) | (hr) |
| Example 5 | IV | 50 | NA | 1480 | 1.48E5* | 0.438* | 106* |
| | | | | (231) | (7.08E3) | (0.0290) | (12.9) |
| | SQ | 50 | 64 | 269 | 8.77E4* | 1.14* | 206* |
| | | | (28) | (56) | (1.89E4) | (0.160) | (102) |
| Example 6 | IV | 25 | NA | 383 | 8390 | 3.00 | 15.7 |
| | | | | (42.4) | (892) | (0.317) | (2.35) |
| | SQ | 25 | 32 | 57.8 | 3240 | 7.96 | 12.6 |
| | | | (14) | (12.0) | (746) | (1.62) | (0.968) |
| Example 7 | IV | 25 | NA | 309 | 5830 | 4.30 | 13.9 |
| | | | | (29.2) | (180) | (0.134) | (4.78) |
| | SQ | 25 | 24 | 31.6 | 1520 | 20.2 | 13.2 |
| | | | (0) | (16.9) | (755) | (11.6) | (3.19) |

COMMENTS:
*The study duration is too short to fully characterize the elimination phase of the PK profile.
The data are mean values (standard deviation in parentheses): Example 5 values are N = 3 (IV and SC from 1-240 hours); Example 6 IV values are N = 3 (1-168 hours) and N = 1 (240 hours), and SC values are N = 3 (6-144 hours); Example 7 IV values are N = 3 (1-96 hours), N = 2 (144 hours), and N = 1 (168, 240 hours); and SC values are N = 3 (6 to 96 hours), N = 2 (144 hours), and N = 1 (168 hours).
NOTE:
Abbreviations: t½ = half-life, Tₘₐₓ = time to maximum concentration, Cₘₐₓ = maximum observed plasma concentration, C₀ = plasma concentration extrapolated to time zero, AUC_{0-inf} = area under the curve from time 0 hours to infinity, CL = clearance, CL/F = clearance/bioavailability.

As shown in Table 29, the VHH fusions of Examples 5, 6 and 7 demonstrate an extended PK profile in Sprague Dawley rats relative to the non-fused proteins (data not shown).

### Example 31: PK of Fab-VHH Fusion of Example 9 and the Corresponding Non-Fused Fab (Example 13) in Male Sprague Dawley Rats

Male Sprague Dawley rats are administered a single intravenous (IV) or subcutaneous (SC) dose of 63.4 nmol/kg for Example 9, 78.9 nmol/kg for Example 13, formulated in PBS buffer (pH 7.4) at a dose volume of 4 mL/kg. Blood is collected for PK characterization at 1, 6, 12, 24, 48, 96, 144, 168 and 240 hr post-dose for IV routes; and at 6, 12, 24, 48, 96, 144, 168 and 240 hr post-dose for SC routes.

Plasma concentrations of the Fab-VHH fusion of Example 9 and the corresponding non-fused Fab of Example 13 are determined by a qualified LC/MS assay at Altascience Company (Laval, Quebec, Canada) using a Q/Exactive Plus mass spectrometer (Thermo Scientific, San Jose, CA). The Fab-VHH fusion of Examples 9 and the Fab of Example 13 and an internal standard are isolated from K3EDTA rat plasma via immunoprecipitation with an anti-human kappa light chain-biotin conjugate and streptavidin-coated magnetic beads, or an anti-VHH-antibody-biotin conjugate and streptavidin-coated magnetic beads. Following wash steps to remove interfering endogenous proteins, the isolated proteins of Examples 9 and 13 are reduced, alkylated and digested with trypsin, and the tryptic peptides are analyzed by LC/MS as a surrogate measure of the intact fusions. The plasma concentrations (Table 30) of Fab-VHH of Example 9 are used to calculate the PK parameters shown in Table 31. The plasma concentrations of the non-fused Fab of Example 13 are too low to calculate PK parameters.

TABLE 30
Mean Plasma Concentration (reported in nmol/L) for Fab-VHH Fusion of
Example 9 and the Corresponding Non-Fused Fab (Example 13) Following
Single IV or SC Dose Administered to Male Sprague Dawley Rats.

| | | Time (hour) | | | | | | | | |
|---|---|---|---|---|---|---|---|---|---|---|
| Compound | Route | 1 | 6 | 12 | 24 | 48 | 96 | 144 | 168 | 240 |
| Example 9 | IV | 1909.8 | 1378.2 | 999.7 | 797.8 | 515.3 | 257.4 | 133.1 | 96.9 | 34.6 |
| | | (35.4) | (93.2) | (19.7) | (40.3) | (44.0) | (10.6) | (17.4) | (14.2) | (5.0) |
| | SC | NA | 28.1 | 79.2 | 159.6 | 229.8 | 183.1 | 103.0 | 85.0 | 46.0 |
| | | | (7.1) | (26.5) | (30.0) | (16.2) | (16.4) | (13.4) | (8.3) | (20.9) |
| Example 13 | IV | 75.7 | 18.8 | 6.9 | 2.7 | BQL | BQL | BQL | BQL | BQL |
| | | (7.1) | (11.3) | (4.2) | (NC) | | | | | |
| | SC | NA | 12.1 | 5.6 | BQL | BQL | BQL | BQL | BQL | BQL |
| | | | (1.2) | (1.2) | | | | | | |

Standard deviation values are included in the table below the mean concentration values, in parentheses.
N = 3/group

As shown in Table 30, the Fab-VHH fusion of Example 9 demonstrates significantly higher mean plasma concentrations in Sprague Dawley rats relative to the corresponding non-fused Fab (Example 13).

TABLE 31
Mean Plasma Pharmacokinetic Parameters for Fab-VHH Fusion of Example 9 Following
Single IV or SC Dose Administered to Male Sprague Dawley Rats.

| | | | | C₀ (IV) | | CL (IV) | |
|---|---|---|---|---|---|---|---|
| | | Dose | Tₘₐₓ | Cₘₐₓ (SC) | AUC_{0-inf} | CL/F (SC) | t½ |
| Compound | Route | (nmol/kg) | (hr) | (nmol/L) | (hr*nmol/L) | (mL/hr/kg) | (hr) |
| Example 9 | IV | 78.9 | NA | 2039 | 8.17E4 | 0.966 | 49.4 |
| | | | | (23) | (3.16E3) | (0.037) | (1.7) |
| | SC | 78.9 | 48 | 230 | 3.56E4 | 2.259 | 73.8 |
| | | | (0) | (16) | (6.24E3) | (0.361) | (20.9) |

NOTE:
Abbreviations: t½ = half-life, Tₘₐₓ = time to maximum concentration, Cₘₐₓ = maximum observed plasma concentration, C₀ = plasma concentration extrapolated to time zero, AUC_{0-inf} = area under the curve from time 0 hours to infinity, CL = clearance, CL/F = clearance/bioavailability.
Standard deviation values are included in the table below the mean PK parameters, in parentheses.
N = 3/group

As shown in Table 31, the Fab-VHH fusion of Example 9 demonstrates an extended PK profile in Sprague Dawley rats while the mean plasma concentrations of the corresponding non-fused Fab (Example 13) are too low to even calculate PK parameters.

### Sequences

The following nucleic and/or amino acid sequences are referred to in the disclosure and are provided below for reference.

VHH moiety 1 (MC6.1C22)
SEQ ID NO: 1
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSS
VHH moiety 2 (MC6.1C80)
SEQ ID NO: 2
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSS
VHH moiety 3 (MC6.1C22.43)
SEQ ID NO: 3
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSPP
VHH moiety 4 (MC6.1C80.43)
SEQ ID NO: 4
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSPP
VHH moiety 5 (MC6)
SEQ ID NO: 5
EVQLVESGGGLVQAGGSLRLSCAASGRTVSSTAVAWFRQAPGKER
EFTAGIGGSVDITYYLDSVKGRFTISKDNTKNTVYLQMNSLKPED
TAVYYCAVRPGRPLITSRDANLYDYWGQGTQVTVSS
VHH moiety 6 (MC6.1)
SEQ ID NO: 6
EVQLLESGGGLVQPGGSLRLSCAASGRTVSSTAVAWFRQAPGKER
EFVAGIGGSVDITYYADSVKGRFTISRDNSKNTLYLQMNSLRPED
TAVYYCAVRPGRPLITSRDANLYDYWGQGTLVTVSS
VHH moiety 7 (MC6.1C6)
SEQ ID NO: 7
EVQLLESGGGLVQPGGSLRLSCAASGRYIDSTAVAWFRQAPGKER
EFVAGIGGGVDITYYADSVKGRFTISRDNSKNTLYLQMNSLRPED
TAVYYCAARPGRPLITSRVANLYPYWGQGTLVTVSS
VHH moiety 8 (MC6.1C22-G26Y)
SEQ ID NO: 8
EVQLLESGGGLVQPGGSLRLSCAASYRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSS
VHH moiety 9 (MC6.1C22-R27A)
SEQ ID NO: 9
EVQLLESGGGLVQPGGSLRLSCAASGAYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSS
VHH moiety 10 (MC6.1C22-157E)
SEQ ID NO: 10
EVQLLESGGGLVQPGGSLRLSCAASGRYIDETAVAWFRQAPGKER
EFVAGIGGGVDETYYADSVKGRFTISRDNSKNTLYLQMNSLRPED
TAVYYCAARPGRPLITSKVADLYPYWGQGTLVTVSS
VHH moiety 11 (MC6.1C22-157Q)
SEQ ID NO: 11
EVQLLESGGGLVQPGGSLRLSCAASGRYIDETAVAWFRQAPGKER
EFVAGIGGGVDQTYYADSVKGRFTISRDNSKNTLYLQMNSLRPED
TAVYYCAARPGRPLITSKVADLYPYWGQGTLVTVSS
VHH moiety 12 (MC6.1C22-Y59A)
SEQ ID NO: 12
EVQLLESGGGLVQPGGSLRLSCAASGRYIDETAVAWFRQAPGKER
EFVAGIGGGVDITAYADSVKGRFTISRDNSKNTLYLQMNSLRPED
TAVYYCAARPGRPLITSKVADLYPYWGQGTLVTVSS
VHH moiety 13 (MC6.1C22-Y59E)
SEQ ID NO: 13
EVQLLESGGGLVQPGGSLRLSCAASGRYIDETAVAWFRQAPGKER
EFVAGIGGGVDITEYADSVKGRFTISRDNSKNTLYLQMNSLRPED
TAVYYCAARPGRPLITSKVADLYPYWGQGTLVTVSS
VHH moiety 14 (MC6.1C22-Y59Q)
SEQ ID NO: 14
EVQLLESGGGLVQPGGSLRLSCAASGRYIDETAVAWFRQAPGKER
EFVAGIGGGVDITQYADSVKGRFTISRDNSKNTLYLQMNSLRPED
TAVYYCAARPGRPLITSKVADLYPYWGQGTLVTVSS
VHH moiety 15 (MC6.1C22-Y59S)
SEQ ID NO: 15
EVQLLESGGGLVQPGGSLRLSCAASGRYIDETAVAWFRQAPGKER
EFVAGIGGGVDITSYADSVKGRFTISRDNSKNTLYLQMNSLRPED
TAVYYCAARPGRPLITSKVADLYPYWGQGTLVTVSS
VHH moiety 16 (MC6.1C22-Y59T)
SEQ ID NO: 16
EVQLLESGGGLVQPGGSLRLSCAASGRYIDETAVAWFRQAPGKER
EFVAGIGGGVDITTYADSVKGRFTISRDNSKNTLYLQMNSLRPED
TAVYYCAARPGRPLITSKVADLYPYWGQGTLVTVSS
VHH moiety 17 (MC6.1C22-R102K)
SEQ ID NO: 17
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGKPLITSKVADLYPYWGQGTLVTVSS
VHH moiety 18 (MC6.1C22-R102Q)
SEQ ID NO: 18
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGQPLITSKVADLYPYWGQGTLVTVSS
VHH moiety 19 (MC6.1C22-R102S)
SEQ ID NO: 19
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGSPLITSKVADLYPYWGQGTLVTVSS
VHH moiety 20 (MC6.1C22-P103)
SEQ ID NO: 20
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRELITSKVADLYPYWGQGTLVTVSS
VHH moiety 21 (MC6.1C22-P103Q)
SEQ ID NO: 21
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRQLITSKVADLYPYWGQGTLVTVSS
VHH moiety 22 (MC6.1C22-P103S)
SEQ ID NO: 22
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRSLITSKVADLYPYWGQGTLVTVSS
VHH moiety 23 (MC6.1C22-L104E)
SEQ ID NO: 23
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPEITSKVADLYPYWGQGTLVTVSS
VHH moiety 24 (MC6.1C22-L104G)
SEQ ID NO: 24
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPGITSKVADLYPYWGQGTLVTVSS
VHH moiety 25 (MC6.1C22-L104Q)
SEQ ID NO: 25
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPQITSKVADLYPYWGQGTLVTVSS
VHH moiety 26 (MC6.1C22-L104T)
SEQ ID NO: 26
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPTITSKVADLYPYWGQGTLVTVSS
VHH moiety 27 (MC6.1C22-S107E)
SEQ ID NO: 27
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITEKVADLYPYWGQGTLVTVSS
VHH moiety 28 (MC6.1C80-A98T)
SEQ ID NO: 28
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCATRPGRPLITSKVADLYPYWGQGTLVTVSS
VHH moiety 29 (MC6.1C80-P100Q)
SEQ ID NO: 29
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARQGRPLITSKVADLYPYWGQGTLVTVSS
VHH moiety 30 (MC6.1C80-A98T, P100Q)
SEQ ID NO: 30
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCATRQGRPLITSKVADLYPYWGQGTLVTVSS
VHH moiety 31 (MC6.1C80-K108Q)
SEQ ID NO: 31
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSQVADLYPYWGQGTLVTVSS
VHH moiety 32 (MC6.1C80-V109Q)
SEQ ID NO: 32
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKQADLYPYWGQGTLVTVSS
VHH moiety 33 (MC6.1C80-D111E)
SEQ ID NO: 33
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVAELYPYWGQGTLVTVSS
VHH moiety 34 (MC6.1C80-D111S)
SEQ ID NO: 34
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVASLYPYWGQGTLVTVSS
VHH moiety 35 (MC6.1C80-V109Q, D111E)
SEQ ID NO: 35
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKQAELYPYWGQGTLVTVSS
VHH moiety 36 (MC6.1C80-V109Q, D111S)
SEQ ID NO: 36
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKQASLYPYWGQGTLVTVSS
VHH moiety 37 (MC6.1C80Cys)
SEQ ID NO: 37
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSC
Li (basic sequence of (GGGGQ)ₙ)
SEQ ID NO: 38
GGGGQ
L₁ (basic sequence of (GGGQ)ₙ)
SEQ ID NO: 39
GGGQ
L₁ (basic sequence of (GGGGS)ₙ)
SEQ ID NO: 40
GGGGS
Li (basic sequence of (PGPQ)ₙ)
SEQ ID NO: 41
PGPQ
L₁ (basic sequence of (PGPA)ₙ)
SEQ ID NO: 42
PGPA
Li (basic sequence of GGGG(AP)ₙGGGG)
SEQ ID NO: 43
GGGG-AP-GGGG
Li (basic sequence of (GGE)ₙ)
SEQ ID NO: 44
GGE
L₁ (basic sequence of (GGGGE)ₙ)
SEQ ID NO: 45
GGGGE
L₁ (basic sequence of (GGK)ₙ)
SEQ ID NO: 46
GGK
L₁ (basic sequence of (GGGGK)ₙ)
SEQ ID NO: 47
GGGGK
L₁ (basic sequence of GGGG(EP)ₙGGGG)
SEQ ID NO: 48
GGGG-EP-GGGG
Li (basic sequence of GGGG(KP) nGGGG)
SEQ ID NO: 49
GGGG-KP-GGGG
L₁ (basic sequence of (PGPE)ₙ)
SEQ ID NO: 50
PGPE
L₁ (basic sequence of (PGPK)ₙ)
SEQ ID NO: 51
PGPK
L₁ 1 (GGGGQ)₅
SEQ ID NO: 52
GGGGQGGGGQGGGGQGGGGQGGGGQ
L₁ 2 (PGPQ)₈
SEQ ID NO: 53
PGPQPGPQPGPQPGPQPGPQPGPQPGPQPGPQ
L₁ 3 (PGPA)₈
SEQ ID NO: 54
PGPAPGP APGPAPGPAPGP APGP APGPAPGPA
L₁ 4 ((GGE)₈)
SEQ ID NO: 55
GGEGGEGGEGGEGGEGGEGGEGGE
L₁ 5 ((GGGGE)₅)
SEQ ID NO: 56
GGGGEGGGGEGGGGEGGGGEGGGGE
L₁ 6 ((GGK)₈)
SEQ ID NO: 57
GGKGGKGGKGGKGGKGGKGGKGGK
L₁ 7 ((GGGGK)₅)
SEQ ID NO: 58
GGGGKGGGGKGGGGKGGGGKGGGGK
L₁ 8 ((GGGG(AP)₁₀GGGG))
SEQ ID NO: 59
GGGGAPAPAPAPAPAPAPAPAPAPGGGG
L₁ 9 ((GGGG(EP)₁₀GGGG))
SEQ ID NO: 60
GGGGEPEPEPEPEPEPEPEPEPEPGGGG
L₁ 10 ((GGGG(KP)₁₀GGGG))
SEQ ID NO: 61
GGGGKPKPKPKPKPKPKPKPKPKPGGGG
L₁ 11 ((PGPE)₈)
SEQ ID NO: 62
PGPEPGPEPGPEPGPEPGPEPGPEPGPEPGPE
L₁ 12 ((PGPK)₈)
SEQ ID NO: 63
PGPKPGPKPGPKPGPKPGPKPGPKPGPKPGPK
L₂ 1
SEQ ID NO: 64
GGGSGGSGGG
L₂ 2
SEQ ID NO: 65
GGGSGGSGGSGGG
signal peptide
SEQ ID NO: 66
METDTLLLWVLLLWVPGSTG
human proGIP
SEQ ID NO: 67
MVATKTFALLLLSLFLAVGLGEKKEGHFSALPSLPVGSHAKVSSP
QPRGPRYAEGTFISDYSIAMDKIHQQDFVNWLLAQKGKKNDWKHN
ITQREARALELASQANRKEEEAVEPQSSPAKNPSDEDLLRDLLIQ
ELLACLLDQTNLCRLRSR
human GIP
SEQ ID NO: 68
YAEGTFISDYSIAMDKIHQQDFVNWLLAQKGKKNDWKHNITQ
human GIP receptor
SEQ ID NO: 69
RAETGSKGQTAGELYQRWERYRRECQETLAAAEPPSGLACNGSFD
MYVCWDYAAPNATARASCPWYLPWHHHVAAGFVLRQCGSDGQWGL
WRDHTQCENPEKNEAFLDQRLILERLQVMYTVGYSLSLATLLLAL
LILSLFRRLHCTRNYIHINLFTSFMLRAAAILSRDRLLPRPGPYL
GDQALALWNQALAACRTAQIVTQYCVGANYTWLLVEGVYLHSLLV
LVGGSEEGHFRYYLLLGWGAPALFVIPWVIVRYLYENTQCWERNE
VKAIWWIIRTPILMTILINFLIFIRILGILLSKLRTRQMRCRDYR
LRLARSTLTLVPLLGVHEVVFAPVTEEQARGALRFAKLGFEIFLS
SFQGFLVSVLYCFINKEVQSEIRRGWHHCRLRRSLGEEQRQLPER
AFRALPSGSGPGEVPTSRGLSSGTLPGPGNEASRELESYC
human proglucagon
SEQ ID NO: 70
MKSIYFVAGLFVMLVQGSWQRSLQDTEEKSRSFSASQADPLSDPD
QMNEDKRHSQGTFTSDYSKYLDSRRAQDFVQWLMNTKRNRNNIAK
RHDEFERHAEGTFTSDVSSYLEGQAAKEFIAWLVKGRGRRDFPEE
VAIVEELGRRHADGSFSDEMNTILDNLAARDFINWLIQTKITDRK
human GLP-1
SEQ ID NO: 71
HDEFERHAEGTFTSDVSSYLEGQAAKEFIAWLVKGRG
human GLP-1₇₋₃₇
SEQ ID NO: 72
HAEGTFTSDVSSYLEGQAAKEFIAWLVKGRG
human GLP-1₇₋₃₆
SEQ ID NO: 73
HAEGTFTSDVSSYLEGQAAKEFIAWLVKGR
human GLP-1 receptor
SEQ ID NO: 74
RPQGATVSLWETVQKWREYRRQCQRSLTEDPPPATDLFCNRTFDE
YACWPDGEPGSFVNVSCPWYLPWASSVPQGHVYRFCTAEGLWLQK
DNSSLPWRDLSECEESKRGERSSPEEQLLFLYIIYTVGYALSFSA
LVIASAILLGFRHLHCTRNYIHLNLFASFILRALSVFIKDAALKW
MYSTAAQQHQWDGLLSYQDSLSCRLVFLLMQYCVAANYYWLLVEG
VYLYTLLAFSVLSEQWIFRLYVSIGWGVPLLFVVPWGIVKYLYED
EGCWTRNSNMNYWLIIRLPILFAIGVNFLIFVRVICIVVSKLKAN
LMCKTDIKCRLAKSTLTLIPLLGTHEVIFAFVMDEHARGTLRFIK
LFTELSFTSFQGLMVAILYCFVNNEVQLEFRKSWERWRLEHLHIQ
RDSSMKPLKCPTSSLSSGATAGSSMYTATCQASCS
human proGDF15
SEQ ID NO: 75
MPGQELRTVNGSQMLLVLLVLSWLPHGGALSLAEASRASFPGPSE
LHSEDSRFRELRKRYEDLLTRLRANQSWEDSNTDLVPAPAVRILT
PEVRLGSGGHLHLRISRAALPEGLPEASRLHRALFRLSPTASRSW
DVTRPLRRQLSLARPQAPALHLRLSPPPSQSDQLLAESSSARPQL
ELHLRPQAARGRRRARARNGDHCPLGPGRCCRLHTVRASLEDLGW
ADWVLSPREVQVTMCIGACPSQFRAANMHAQIKTSLHRLKPDTVP
APCCVPASYNPMVLIQKTDTGVSLQTYDDLLAKDCHCI
human GDF15
SEQ ID NO: 76
ARNGDHCPLGPGRCCRLHTVRASLEDLGWADWVLSPREVQVTMCI
GACPSQFRAANMHAQIKTSLHRLKPDTVPAPCCVPASYNPMVLIQ
KTDTGVSLQTYDDLLAKDCHCI
human GDF15 (GFRAL) receptor
SEQ ID NO: 77
MIVFIFLAMGLSLENEYTSQTNNCTYLREQCLRDANGCKHAWRVM
EDACNDSDPGDPCKMRNSSYCNLSIQYLVESNFQFKECLCTDDFY
CTVNKLLGKKCINKSDNVKEDKFKWNLTTRSHHGFKGMWSCLEVA
EACVGDVVCNAQLASYLKACSANGNPCDLKQCQAAIRFFYQNIPF
NIAQMLAFCDCAQSDIPCQQSKEALHSKTCAVNMVPPPTCLSVIR
SCQNDELCRRHYRTFQSKCWQRVTRKCHEDENCISTLSKQDLTCS
GSDDCKAAYIDILGTVLQVQCTCRTITQSEESLCKIFQHMLHRKS
CFNYPTLSNVKGMALYTRKHANKITLTGFHSPFNGEVIYAAMCMT
VTCGILLLVMVKLRTSRISSKARDPSSIQIPGEL
human proINS
SEQ ID NO: 78
FVNQHLCGSHLVEALYLVCGERGFFYTPKTRREAEDLQVGQVELG
GGPGAGSLQPLALEGSLQKRGIVEQCCTSICSLYQLENYCN
human INS A chain
SEQ ID NO: 79
GIVEQCCTSICSLYQLENYCN
human INS B chain
SEQ ID NO: 80
FVNQHLCGSHLVEALYLVCGERGFFYTPKT
human INS receptor α-subunit
SEQ ID NO: 81
HLYPGEVCPGMDIRNNLTRLHELENCSVIEGHLQILLMFKTRPED
FRDLSFPKLIMITDYLLLFRVYGLESLKDLFPNLTVIRGSRLFFN
YALVIFEMVHLKELGLYNLMNITRGSVRIEKNNELCYLATIDWSR
ILDSVEDNYIVLNKDDNEECGDICPGTAKGKTNCPATVINGQFVE
RCWTHSHCQKVCPTICKSHGCTAEGLCCHSECLGNCSQPDDPTKC
VACRNFYLDGRCVETCPPPYYHFQDWRCVNFSFCQDLHHKCKNSR
RQGCHQYVIHNNKCIPECPSGYTMNSSNLLCTPCLGPCPKVCHLL
EGEKTIDSVTSAQELRGCTVINGSLIINIRGGNNLAAELEANLGL
IEEISGYLKIRRSYALVSLSFFRKLRLIRGETLEIGNYSFYALDN
QNLRQLWDWSKHNLTITQGKLFFHYNPKLCLSEIHKMEEVSGTKG
RQERNDIALKTNGDQASCENELLKFSYIRTSFDKILLRWEPYWPP
DFRDLLGFMLFYKEAPYQNVTEFDGQDACGSNSWTVVDIDPPLRS
NDPKSQNHPGWLMRGLKPWTQYAIFVKTLVTFSDERRTYGAKSDI
IYVQTDATNPSVPLDPISVSNSSSQIILKWKPPSDPNGNITHYLV
FWERQAEDSELFELDYCLKGLKLPSRTWSPPFESEDSQKHNQSEY
EDSAGECCSCPKTDSQILKELEESSFRKTFEDYLHNVVFVPRKTS
SGTGAEDPRPS
human INS receptor β-subunit
SEQ ID NO: 82
SLGDVGNVTVAVPTVAAFPNTSSTSVPTSPEEHRPFEKVVNKESL
VISGLRHFTGYRIELQACNQDTPEERCSVAAYVSARTMPEAKADD
IVGPVTHEIFENNVVHLMWQEPKEPNGLIVLYEVSYRRYGDEELH
LCVSRKHFALERGCRLRGLSPGNYSVRIRATSLAGNGSWTEPTYF
YVTDYLDVPSNIAKIIIGPLIFVFLFSVVIGSIYLFLRKRQPDGP
LGPLYASSNPEYLSASDVFPCSVYVPDEWEVSREKITLLRELGQG
SFGMVYEGNARDIIKGEAETRVAVKTVNESASLRERIEFLNEASV
MKGFTCHHVVRLLGVVSKGQPTLVVMELMAHGDLKSYLRSLRPEA
ENNPGRPPPTLQEMIQMAAEIADGMAYLNAKKFVHRDLAARNCMV
AHDFTVKIGDFGMTRDIYETDYYRKGGKGLLPVRWMAPESLKDGV
FTTSSDMWSFGVVLWEITSLAEQPYQGLSNEQVLKFVMDGGYLDQ
PDNCPERVTDLMRMCWQFNPKMRPTFLEIVNLLKDDLHPSFPEVS
FFHSEENKAPESEELEMEFEDMENVPLDRSSHCQREEAGGRDGGS
SLGFKRSYEEHIPYTHMNGGKKNGRILTLPRSNPS
human IL-2
SEQ ID NO: 83
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
human IL-2 receptor α-subunit
SEQ ID NO: 84
ELCDDDPPEIPHATFKAMAYKEGTMLNCECKRGFRRIKSGSLYML
CTGNSSHSSWDNQCQCTSSATRNTTKQVTPQPEEQKERKTTEMQS
PMQPVDQASLPGHCREPPPWENEATERIYHFVVGQMVYYQCVQGY
RALHRGPAESVCKMTHGKTRWTQPQLICTGEMETSQFPGEEKPQA
SPEGRPESETSCLVTTTDFQIQTEMAATMETSIFTTEYQVAVAGC
VFLLISVLLLSGLTWQRRQRKSRRTI
human IL-2 receptor β-subunit
SEQ ID NO: 85
AVNGTSQFTCFYNSRANISCVWSQDGALQDTSCQVHAWPDRRRWN
QTCELLPVSQASWACNLILGAPDSQKLTTVDIVTLRVLCREGVRW
RVMAIQDFKPFENLRLMAPISLQVVHVETHRCNISWEISQASHYF
ERHLEFEARTLSPGHTWEEAPLLTLKQKQEWICLETLTPDTQYEF
QVRVKPLQGEFTTWSPWSQPLAFRTKPAALGKDTIPWLGHLLVGL
SGAFGFIILVYLLINCRNTGPWLKKVLKCNTPDPSKFFSQLSSEH
GGDVQKWLSSPFPSSSFSPGGLAPEISPLEVLERDKVTQLLLQQD
KVPEPASLSSNHSLTSCFTNQGYFFFHLPDALEIEACQVYFTYDP
YSEEDPDEGVAGAPTGSSPQPLQPLSGEDDAYCTFPSRDDLLLFS
PSLLGGPSPPSTAPGGSGAGEERMPPSLQERVPRDWDPQPLGPPT
PGVPDLVDFQPPPELVLREAGEEVPDAGPREGVSFPWSRPPGQGE
FRALNARLPLNTDAYLSLQELQGQDPTHLV
human IL-2 receptor γ-subunit
SEQ ID NO: 86
LNTTILTPNGNEDTTADFFLTTMPTDSLSVSTLPLPEVQCFVFNV
EYMNCTWNSSSEPQPTNLTLHYWYKNSDNDKVQKCSHYLFSEEIT
SGCQLQKKEIHLYQTFVVQLQDPREPRRQATQMLKLQNLVIPWAP
ENLTLHKLSESQLELNWNNRFLNHCLEHLVQYRTDWDHSWTEQSV
DYRHKFSLPSVDGQKRYTFRVRSRFNPLCGSAQHWSEWSHPIHWG
SNTSKENPFLFALEAVVISVGSMGLIISLLCVYFWLERTMPRIPT
LKNLEDLVTEYHGNFSAWSGVSKGLAESLQPDYSERLCLVSEIPP
KGGALGEGPGASPCNQHSPYWAPPCYTLKPET
human proNRG1
SEQ ID NO: 87
MSERKEGRGKGKGKKKERGSGKKPESAAGSQSPALPPRLKEMKSQ
ESAAGSKLVLRCETSSEYSSLRFKWFKNGNELNRKNKPQNIKIQK
KPGKSELRINKASLADSGEYMCKVISKLGNDSASANITIVESNEI
ITGMPASTEGAYVSSESPIRISVSTEGANTSSSTSTSTTGTSHLV
KCAEKEKTFCVNGGECFMVKDLSNPSRYLCKCQPGFTGARCTENV
PMKVQNQEKAEELYQKRVLTITGICIALLVVGIMCVVAYCKTKKQ
RKKLHDRLRQSLRSERNNMMNIANGPHHPNPPPENVQLVNQYVSK
NVISSEHIVEREAETSFSTSHYTSTAHHSTTVTQTPSHSWSNGHT
ESILSESHSVIVMSSVENSRHSSPTGGPRGRLNGTGGPRECNSFL
RHARETPDSYRDSPHSERYVSAMTTPARMSPVDFHTPSSPKSPPS
EMSPPVSSMTVSMPSMAVSPFMEEERPLLLVTPPRLREKKFDHHP
QQFSSFHHNPAHDSNSLPASPLRIVEDEEYETTQEYEPAQEPVKK
LANSRRAKRTKPNGHIANRLEVDSNTSSQSSNSESETEDERVGED
TPFLGIQNPLAASLEATPAFRLADSRTNPAGRFSTQEEIQARLSS
VIANQDPIAV
human NRG1
SEQ ID NO: 88
SGKKPESAAGSQSPALPPRLKEMKSQESAAGSKLVLRCETSSEYS
SLRFKWFKNGNELNRKNKPQNIKIQKKPGKSELRINKASLADSGE
YMCKVISKLGNDSASANITIVESNEIITGMPASTEGAYVSSESPI
RISVSTEGANTSSSTSTSTTGTSHLVKCAEKEKTFCVNGGECFMV
KDLSNPSRYLCKCQPGFTGARCTENVPMKVQNQEKAEELYQK
human NRG1 receptor ErbB3
SEQ ID NO: 89
MRANDALQVLGLLFSLARGSEVGNSQAVCPGTLNGLSVTGDAENQ
YQTLYKLYERCEVVMGNLEIVLTGHNADLSFLQWIREVTGYVLVA
MNEFSTLPLPNLRVVRGTQVYDGKFAIFVMLNYNTNSSHALRQLR
LTQLTEILSGGVYIEKNDKLCHMDTIDWRDIVRDRDAEIVVKDNG
RSCPPCHEVCKGRCWGPGSEDCQTLTKTICAPQCNGHCFGPNPNQ
CCHDECAGGCSGPQDTDCFACRHFNDSGACVPRCPQPLVYNKLTF
QLEPNPHTKYQYGGVCVASCPHNFVVDQTSCVRACPPDKMEVDKN
GLKMCEPCGGLCPKACEGTGSGSRFQTVDSSNIDGFVNCTKILGN
LDFLITGLNGDPWHKIPALDPEKLNVFRTVREITGYLNIQSWPPH
MHNFSVFSNLTTIGGRSLYNRGFSLLIMKNLNVTSLGFRSLKEIS
AGRIYISANRQLCYHHSLNWTKVLRGPTEERLDIKHNRPRRDCVA
EGKVCDPLCSSGGCWGPGPGQCLSCRNYSRGGVCVTHCNFLNGEP
REFAHEAECFSCHPECQPMEGTATCNGSGSDTCAQCAHFRDGPHC
VSSCPHGVLGAKGPIYKYPDVQNECRPCHENCTQGCKGPELQDCL
GQTLVLIGKTHLTMALTVIAGLVVIFMMLGGTFLYWRGRRIQNKR
AMRRYLERGESIEPLDPSEKANKVLARIFKETELRKLKVLGSGVF
GTVHKGVWIPEGESIKIPVCIKVIEDKSGRQSFQAVTDHMLAIGS
LDHAHIVRLLGLCPGSSLQLVTQYLPLGSLLDHVRQHRGALGPQL
LLNWGVQIAKGMYYLEEHGMVHRNLAARNVLLKSPSQVQVADFGV
ADLLPPDDKQLLYSEAKTPIKWMALESIHFGKYTHQSDVWSYGVT
VWELMTFGAEPYAGLRLAEVPDLLEKGERLAQPQICTIDVYMVMV
KCWMIDENIRPTFKELANEFTRMARDPPRYLVIKRESGPGIAPGP
EPHGLTNKKLEEVELEPELDLDLDLEAEEDNLATTTLGSALSLPV
GTLNRPRGSQSLLSPSSGYMPMNQGNLGESCQESAVSGSSERCPR
PVSLHPMPRGCLASESSEGHVTGSEAELQEKVSMCRSRSRSRSPR
PRGDSAYHSQRHSLLTPVTPLSPPGLEEEDVNGYVMPDTHLKGTP
SSREGTLSSVGLSSVLGTEEEDEDEEYEYMNRRRRHSPPHPPRPS
SLEELGYEYMDVGSDLSASLGSTQSCPLHPVPIMPTAGTTPDEDY
EYMNRQRDGGGPGGDYAAMGACPASEQGYEEMRAFQGPGHQAPHV
HYARLKTLRSLEATDSAFDNPDYWHSRLFPKANAQRT
human NRG1 receptor ErbB4
SEQ ID NO: 90
MKPATGLWVWVSLLVAAGTVQPSDSQSVCAGTENKLSSLSDLEQQ
YRALRKYYENCEVVMGNLEITSIEHNRDLSFLRSVREVTGYVLVA
LNQFRYLPLENLRIIRGTKLYEDRYALAIFLNYRKDGNFGLQELG
LKNLTEILNGGVYVDQNKFLCYADTIHWQDIVRNPWPSNLTLVST
NGSSGCGRCHKSCTGRCWGPTENHCQTLTRTVCAEQCDGRCYGPY
VSDCCHRECAGGCSGPKDTDCFACMNFNDSGACVTQCPQTFVYNP
TTFQLEHNFNAKYTYGAFCVKKCPHNFVVDSSSCVRACPSSKMEV
EENGIKMCKPCTDICPKACDGIGTGSLMSAQTVDSSNIDKFINCT
KINGNLIFLVTGIHGDPYNAIEAIDPEKLNVFRTVREITGFLNIQ
SWPPNMTDFSVFSNLVTIGGRVLYSGLSLLILKQQGITSLQFQSL
KEISAGNIYITDNSNLCYYHTINWTTLFSTINQRIVIRDNRKAEN
CTAEGMVCNHLCSSDGCWGPGPDQCLSCRRFSRGRICIESCNLYD
GEFREFENGSICVECDPQCEKMEDGLLTCHGPGPDNCTKCSHFKD
GPNCVEKCPDGLQGANSFIFKYADPDRECHPCHPNCTQGCNGPTS
HDCIYYPWTGHSTLPQHARTPLIAAGVIGGLFILVIVGLTFAVYV
RRKSIKKKRALRRFLETELVEPLTPSGTAPNQAQLRILKETELKR
VKVLGSGAFGTVYKGIWVPEGETVKIPVAIKILNETTGPKANVEF
MDEALIMASMDHPHLVRLLGVCLSPTIQLVTQLMPHGCLLEYVHE
HKDNIGSQLLLNWCVQIAKGMMYLEERRLVHRDLAARNVLVKSPN
HVKITDFGLARLLEGDEKEYNADGGKMPIKWMALECIHYRKFTHQ
SDVWSYGVTIWELMTFGGKPYDGIPTREIPDLLEKGERLPQPPIC
TIDVYMVMVKCWMIDADSRPKFKELAAEFSRMARDPQRYLVIQGD
DRMKLPSPNDSKFFQNLLDEEDLEDMMDAEEYLVPQAFNIPPPIY
TSRARIDSNRSEIGHSPPPAYTPMSGNQFVYRDGGFAAEQGVSVP
YRAPTSTIPEAPVAQGATAEIFDDSCCNGTLRKPVAPHVQEDSST
QRYSADPTVFAPERSPRGELDEEGYMTPMRDKPKQEYLNPVEENP
FVSRRKNGDLQALDNPEYHNASNGPPKAEDEYVNEPLYLNTFANT
LGKAEYLKNNILSMPEKAKKAFDNPDYWNHSLPPRSTLQHPDYLQ
EYSTKYFYKQNGRIRPIVAENPEYLSEFSLKPGTVLPPPPYRHRN
TVV
human proRLN2
SEQ ID NO: 91
MPRLFFFHLLGVCLLLNQFSRAVADSWMEEVIKLCGRELVRAQIA
ICGMSTWSKRSLSQEDAPQTPRPVAEIVPSFINKDTETINMMSEF
VANLPQELKLTLSEMQPALPQLQQHVPVLKDSSLLFEEFKKLIRN
RQSEAADSSPSELKYLGLDTHSRKKRQLYSALANKCCHVGCTKRS
LARFC
human RLN2 A chain
SEQ ID NO: 92
QLYSALANKCCHVGCTKRSLARFC
human RLN2 B chain
SEQ ID NO: 93
DSWMEEVIKLCGRELVRAQIAICGMSTWS
human RXFP1 receptor
SEQ ID NO: 94
MTSGSVFFYILIFGKYFSHGGGQDVKCSLGYFPCGNITKCLPQLL
HCNGVDDCGNQADEDNCGDNNGWSLQFDKYFASYYKMTSQYPFEA
ETPECLVGSVPVQCLCQGLELDCDETNLRAVPSVSSNVTAMSLQW
NLIRKLPPDCFKNYHDLQKLYLQNNKITSISIYAFRGLNSLTKLY
LSHNRITFLKPGVFEDLHRLEWLIIEDNHLSRISPPTFYGLNSLI
LLVLMNNVLTRLPDKPLCQHMPRLHWLDLEGNHIHNLRNLTFISC
SNLTVLVMRKNKINHLNENTFAPLQKLDELDLGSNKIENLPPLIF
KDLKELSQLNLSYNPIQKIQANQFDYLVKLKSLSLEGIEISNIQQ
RMFRPLMNLSHIYFKKFQYCGYAPHVRSCKPNTDGISSLENLLAS
IIQRVFVWVVSAVTCFGNIFVICMRPYIRSENKLYAMSIISLCCA
DCLMGIYLFVIGGFDLKFRGEYNKHAQLWMESTHCQLVGSLAILS
TEVSVLLLTFLTLEKYICIVYPFRCVRPGKCRTITVLILIWITGF
IVAFIPLSNKEFFKNYYGTNGVCFPLHSEDTESIGAQIYSVAIFL
GINLAAFIIIVFSYGSMFYSVHQSAITATEIRNQVKKEMILAKRF
FFIVFTDALCWIPIFVVKFLSLLQVEIPGTITSWVVIFILPINSA
LNPILYTLTTRPFKEMIHRFWYNYRQRKSMDSKGQKTYAPSFIWV
EMWPLQEMPPELMKPDLFTYPCEMSLISQSTRLNSYS
human ACTH
SEQ ID NO: 95
SYSMEHFRWGKPVGKKRRPVKVYPNGAEDESAEAFPLEF
human ACTH receptor (ACTHR, MC2R)
SEQ ID NO: 96
MKHIINSYENINNTARNNSDCPRVVLPEEIFFTISIVGVLENLIV
LLAVFKNKNLQAPMYFFICSLAISDMLGSLYKILENILIILRNMG
YLKPRGSFETTADDIIDSLFVLSLLGSIFSLSVIAADRYITIFHA
LRYHSIVTMRRTVVVLTVIWTFCTGTGITMVIFSHHVPTVITFTS
LFPLMLVFILCLYVHMFLLARSHTRKISTLPRANMKGAITLTILL
GVFIFCWAPFVLHVLLMTFCPSNPYCACYMSLFQVNGMLIMCNAV
IDPFIYAFRSPELRDAFKKMIFCSRYW
human CNTF
SEQ ID NO: 97
MAFTEHSPLTPHRRDLCSRSIWLARKIRSDLTALTESYVKHQGLN
KNINLDSADGMPVASTDQWSELTEAERLQENLQAYRTFHVLLARL
LEDQQVHFTPTEGDFHQAIHTLLLQVAAFAYQIEELMILLEYKIP
RNEADGMPINVGDGGLFEKKLWGLKVLQELSQWTVRSIHDLRFIS
SHQTGIPARGSHYIANNKKM
human CNTF receptor alpha
SEQ ID NO: 98
QRHSPQEAPHVQYERLGSDVTLPCGTANWDAAVTWRVNGTDLAPD
LLNGSQLVLHGLELGHSGLYACFHRDSWHLRHQVLLHVGLPPREP
VLSCRSNTYPKGFYCSWHLPTPTYIPNTFNVTVLHGSKIMVCEKD
PALKNRCHIRYMHLFSTIKYKVSISVSNALGHNATAITFDEFTIV
KPDPPENVVARPVPSNPRRLEVTWQTPSTWPDPESFPLKFFLRYR
PLILDQWQHVELSDGTAHTITDAYAGKEYIIQVAAKDNEIGTWSD
WSVAAHATPWTEEPRHLTTEAQAAETTTSTTSSLAPPPTTKICDP
GELGS
GLP1-standard
SEQ ID NO: 99
HGEGTFTSDVSSYLEEQAAKEFIAWLVKGRGGGGGSGGGGSGGGG
SESKYGPPCPPCPAPEAAGGPSVFLFPPKPKDTLMISRTPEVTCV
VVDVSQEDPEVQFNWYVDGVEVHNAKTKPREEQFNSTYRVVSVLT
VLHQDWLNGKEYKCKVSNKGLPSSIEKTISKAKGQPREPQVYTLP
PSQEEMTKNQVSLTCLVKGFYPSDIAVEWESNGQPENNYKTTPPV
LDSDGSFFLYSRLTVDKSRWQEGNVFSCSVMHEALHNHYTQKSLS
LSLG
VHH fusion 1 (CNTF-(G₄Q)₅-MC6.1C22.43)
SEQ ID NO: 100
MAFTEHSPLTPHRRDLASRSIWLARKIRSDLTALTESYVKHQGLN
KNINLDSADGMPVASTDRWSELTEAERLQENLQAYRTFHVLLARL
LEDQQVHFTPTEGDFHQAIHTLLLQVAAFAYQIEELMILLEYKIP
RNEADGMPINVGDGGLFEKKLWGLKVLQELSQWTVRSIHDLRFIS
SHQTGGGGGQGGGGQGGGGQGGGGQGGGGQEVQLLESGGGLVQPG
GSLRLSCAASGRYIDETAVAWFRQAPGKEREFVAGIGGGVDITYY
ADSVKGRFTISRDNSKNTLYLQMNSLRPEDTAVYYCAARPGRPLI
TSKVADLYPYWGQGTLVTVSSPP
VHH fusion 2 (MC6.1C22-(G₄Q)₅-CNTF)
SEQ ID NO: 101
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQMAFTEHSPLTPHRRDLASRSIWLARKIRS
DLTALTESYVKHQGLNKNINLDSADGMPVASTDRWSELTEAERLQ
ENLQAYRTFHVLLARLLEDQQVHFTPTEGDFHQAIHTLLLQVAAF
AYQIEELMILLEYKIPRNEADGMPINVGDGGLFEKKLWGLKVLQE
LSQWTVRSIHDLRFISSHQTG
VHH fusion 3 (NRG1-(G₄Q)₅-MC6.1C22.43)
SEQ ID NO: 102
SHLVKCAEKEKTFCVNGGECFMVKDLSNPSRYLCKCPNEFTGDRC
QNYVMASFYKHLGIEFMEAEELYQGGGGQGGGGQGGGGQGGGGQG
GGGQEVQLLESGGGLVQPGGSLRLSCAASGRYIDETAVAWFRQAP
GKEREFVAGIGGGVDITYYADSVKGRFTISRDNSKNTLYLQMNSL
RPEDTAVYYCAARPGRPLITSKVADLYPYWGQGTLVTVSSPP
VHH fusion 4 (MC6.1C22-(G₄Q)₅-NRG1)
SEQ ID NO: 103
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSHLVKCAEKEKTFCVNGGECFMVKDLSNP
SRYLCKCPNEFTGDRCQNYVMASFYKHLGIEFMEAEELYQ
VHH fusion 5 (MC6.1C22-(G₄Q)₅-GDF15)
SEQ ID NO: 104
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQGDHCPLGPGRCCRLHTVRASLEDLGWADW
VLSPREVQVTMCIGACPSQFRAANMHAQIKTSLHRLKPDTVPAPC
CVPASYNPMVLIQKTDTGVSLQTYDDLLAKDCHCI
VHH fusion 6 (IL2-(G₄Q)₅-MC6.1C22.43)
SEQ ID NO: 105
APQSSSTQQTQLQLEHLLLDLQMILNGINNYKNPKLTRMLTFKFY
MPKKATELKHLQCLEEELKPLEEVLNLAQSKNFHLRPRDLISRIN
VIVLELKGSETTFMCEYADETATIVEFLNRWITFAQSIISTLTGG
GGQGGGGQGGGGQGGGGQGGGGQEVQLLESGGGLVQPGGSLRLSC
AASGRYIDETAVAWFRQAPGKEREFVAGIGGGVDITYYADSVKGR
FTISRDNSKNTLYLQMNSLRPEDTAVYYCAARPGRPLITSKVADL
YPYWGQGTLVTVSSPP
VHH fusion 7 (MC6.1C22-(G₄Q)₅-IL2)
SEQ ID NO: 106
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQAPQSSSTQQTQLQLEHLLLDLQMILNGIN
NYKNPKLTRMLTFKFYMPKKATELKHLQCLEEELKPLEEVLNLAQ
SKNFHLRPRDLISRINVIVLELKGSETTFMCEYADETATIVEFLN
RWITFAQSIISTLT
VHH fusion 8 (GLP1-(G₄Q)₅-MC6.1C22.43)
SEQ ID NO: 107
HGEGTFTSDVSSYLEEQAAKEFIAWLVKGGGGGGGQGGGGQGGGG
QGGGGQGGGGQEVQLLESGGGLVQPGGSLRLSCAASGRYIDETAV
AWFRQAPGKEREFVAGIGGGVDITYYADSVKGRFTISRDNSKNTL
YLQMNSLRPEDTAVYYCAARPGRPLITSKVADLYPYWGQGTLVTV
SSPP
HC-VHH (AdaFab VHCH₁-(G₄Q)₅-MC6.1C22.43)
SEQ ID NO: 108
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTGGGGQGGGGQGGGGQGGGGQGGGGQEVQLLESGGGLVQPGG
SLRLSCAASGRYIDETAVAWFRQAPGKEREFVAGIGGGVDITYYA
DSVKGRFTISRDNSKNTLYLQMNSLRPEDTAVYYCAARPGRPLIT
SKVADLYPYWGQGTLVTVSSPP
LC (AdaFab VLCL)
SEQ ID NO: 109
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
HC (AdaFab VHCH₁)
SEQ ID NO: 110
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
LC-VHH (AdaFab VLCL-(G₄Q)₅-MC6.1C22.43)
SEQ ID NO: 111
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGQGGGGQG
GGGQGGGGQGGGGQEVQLLESGGGLVQPGGSLRLSCAASGRYIDE
TAVAWFRQAPGKEREFVAGIGGGVDITYYADSVKGRFTISRDNSK
NTLYLQMNSLRPEDTAVYYCAARPGRPLITSKVADLYPYWGQGTL
VTVSSPP
VHH-HC (MC6.1C22-(G₄Q)₅-AdaFab VHCH₁)
SEQ ID NO: 112
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQEVQLVESGGGLVQPGRSLRLSCAASGFTF
DDYAMHWVRQAPGKGLEWVSAITWNSGHIDYADSVEGRFTISRDN
AKNSLYLQMNSLRAEDTAVYYCAKVSYLSTASSLDYWGQGTLVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCD
LC (AdaFab VLCL)
SEQ ID NO: 113
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
HC (AdaFab VHCH₁)
SEQ ID NO: 114
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
VHH-LC (MC6.1C22-(G₄Q)₅-AdaFab VLCL)
SEQ ID NO: 115
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQDIQMTQSPSSLSASVGDRVTITCRASQGI
RNYLAWYQQKPGKAPKLLIYAASTLQSGVPSRFSGSGSGTDFTLT
ISSLQPEDVATYYCQRYNRAPYTFGQGTKVEIKRTVAAPSVFIFP
PSDEQLKSGTASVVCLLNNFYPREAKVQWKVDNALQSGNSQESVT
EQDSKDSTYSLSSTLTLSKADYEKHKVYACEVTHQGLSSPVTKSF
NRGEC
VHH Fusion 13 (GLP1-MC6.1C80Cys)
SEQ ID NO: 116
HGEGTFTSDVSSYLEEQAAKEFIAWLVKGGGGGGGQGGGGQGGGG
QGGGGQGGGGQEVQLLESGGGLVQPGGSLRLSCAASGRYIDETAV
AWFRQAPGKGREFVAGIGGGVDITYYADSVKGRFTISRDNSKNTL
YLQMNSLRPEDTAVYYCAARPGRPLITSKVADLYPYWGQGTLVTV
SSC
VHH fusion conjugate 1
(GLP1-C80Cys-ACTH conjugate)
SEQ ID NO: 117
HGEGTFTSDVSSYLEEQAAKEFIAWLVKGGGGGGGQGGGGQGGGG
QGGGGQGGGGQEVQLLESGGGLVQPGGSLRLSCAASGRYIDETAV
AWFRQAPGKGREFVAGIGGGVDITYYADSVKGRFTISRDNSKNTL
YLQMNSLRPEDTAVYYCAARPGRPLITSKVADLYPYWGQGTLVTV
SSC-MALEIMIDE-(PEG)₁₂-ACTH
(in a C-terminal to N-terminal orientation)

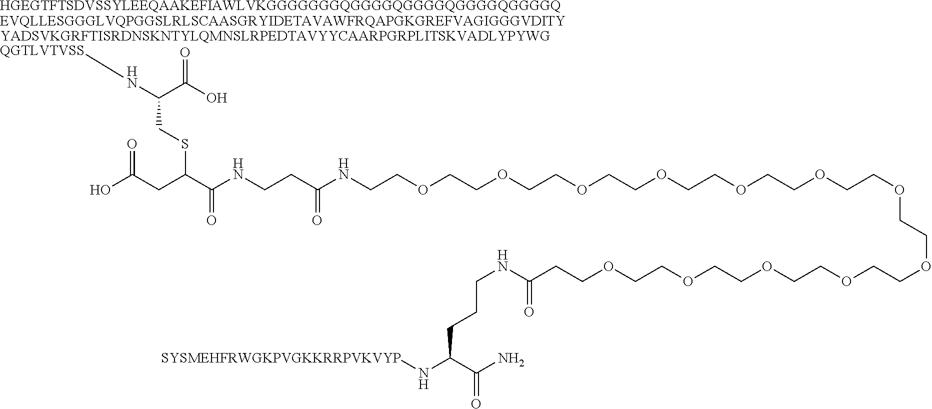

VHH fusion conjugate 2 (MC6.1C80Cys-
ACTH conjugate)
SEQ ID NO: 118
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSC-
maleimide-(PEG)₁₂-ACTH
(in a C-terminal to N-terminal
orientation)

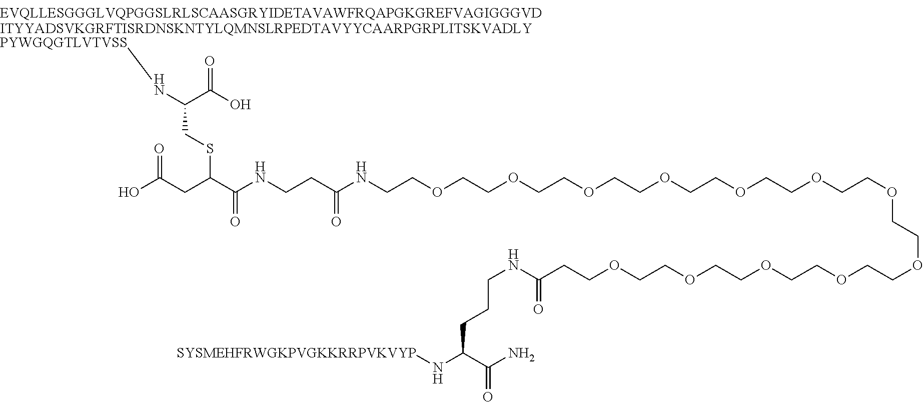

Intermediate 1
(ACTH-(PEG)₁₂-maleimide conjugate)
SEQ ID NO: 119
SYSMEHFRWGKPVGKKRRPVKVYPK-(PEG)₁₂-
maleimide-NH₂

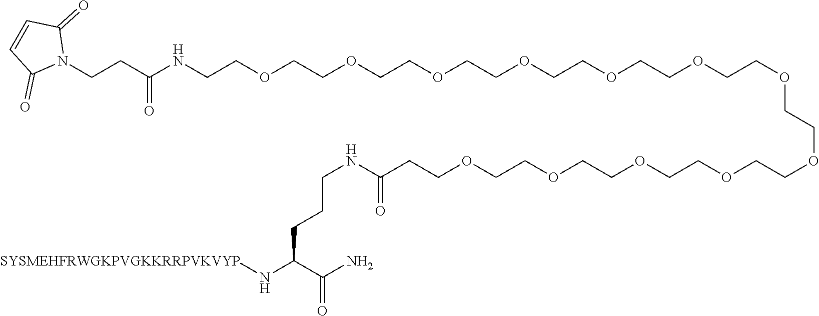

Fab HC (AdaFab VHCH₁)
SEQ ID NO: 120
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
Fab LC (AdaFab VLCL)
SEQ ID NO: 121
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Human GITR full-length (w/o signal peptide)
SEQ ID NO: 122
QRPTGGPGCGPGRLLLGTGTDARCCRVHTTRCCRDYPGEECCSEW
DCMCVQPEFHCGDPCCTTCRHHPCPPGQGVQSQGKFSFGFQCIDC
ASGTFSGGHEGHCKPWTDCTQFGFLTVFPGNKTHNAVCVPGSPPA
EPLGWLTVVLLAVAACVLLLTSAQLGLHIWQLRSQCMWPRETQLL
LEVPPSTEDARSCQFPEEERGERSAEEKGRLGDLWV
Human GITR ECD (w/o signal peptide)
SEQ ID NO: 123
QRPTGGPGCGPGRLLLGTGTDARCCRVHTTRCCRDYPGEECCSEW
DCMCVQPEFHCGDPCCTTCRHHPCPPGQGVQSQGKFSFGFQCIDC
ASGTFSGGHEGHCKPWTDCTQFGFLTVFPGNKTHNAVCVPGSPPA
E
VHH moiety 38 (MC6.1C90Cys)
SEQ ID NO: 124
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCGARPGRPLITSKVADLYPYWGQGTLVTVSSC
VHH moiety 39 (MC6.1C90.43)
SEQ ID NO: 125
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCGARPGRPLITSKVADLYPYWGQGTLVTVSSPP
VHH moiety 40 (MC6.1C95.43)
SEQ ID NO: 126
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCGTRPGRPLITSKVADLYPYWGQGTLVTVSSPP

The invention claimed is:

1. A compound comprising an amino acid sequence selected from the group consisting of SEQ ID NOS:1-37 and 124-126, or at least about 90% sequence similarity thereto.

2-41. (canceled)

42. A compound comprising a structure of:
M-X₁,
X₁-M,
M-L₁-X₁,
X₁-L₁-M,
X₁-M-X₂,
X₂-M-X₁,
X₁-L₁-M-X₂,
X₂-L₁-M-X₁,
X₁-M-L₁-X₂,
X₂-M-L₁-X₁,
X₁-L₁-M-L₂-X₂,
X₂-L₁-M-L₂-X₁,
M-L₁-X₁-L₂-X₂,
M-L₁-X₂-L₂-X₁,
X₁-L₂-X₂-L₁-M, or
X₂-L₂-X₁-L₁-M,
wherein M is an amino acid sequence selected from the group consisting of SEQ ID NOS:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 124, 125 and 126, or at least about 90% sequence similarity thereto,
wherein X₁ is a first biotherapeutic,
wherein X₂ (if present) is a second biotherapeutic,
wherein L₁ (if present) is a first linker having an amino acid sequence selected from the group consisting of (GGGGQ)ₙ (SEQ ID NO:38), (GGGQ)ₙ (SEQ ID NO:39), (GGGGS)ₙ (SEQ ID NO:40), (PGPQ)ₙ (SEQ ID NO:41), (PGPA)ₙ (SEQ ID NO:42), (GGGG(AP)ₙGGGG) (SEQ ID NO:43), (GGE)ₙ (SEQ ID NO:44), (GGGGGE)ₙ (SEQ ID NO:45), (GGK)ₙ (SEQ ID NO:46), (GGGGK)ₙ (SEQ ID NO:47), (GGGG(EP)ₙGGGG) (SEQ ID NO:48), (GGGG(KP)ₙGGGG) (SEQ ID NO:49), (PGPE)ₙ (SEQ ID NO:50), and (PGPK)ₙ (SEQ ID NO:51), wherein n can be from 1 to 10, and
wherein L₂ (if present) is a second linker having an amino acid sequence selected from the group consisting of SEQ ID NOS:64 and 65, or at least about 90% sequence similarity thereto.

43. The compound of claim 42, wherein the first biotherapeutic is a peptide, protein or oligomer.

44. The compound of claim 42, wherein the second biotherapeutic is a peptide, protein or oligomer.

45. The compound of claim 42, wherein the first biotherapeutic and the second biotherapeutic are distinct from one another.

46. The compound of claim 42, wherein L₁ has an amino acid sequence selected from the group consisting of SEQ ID NOS:52 to 63, or at least about 90% sequence similarity thereto.

47. The compound of claim 42, wherein the first biotherapeutic and/or the second biotherapeutic is a peptide or protein such as an antibody (Ab) or fragment thereof, adrenocorticotropic hormone (ACTH), growth/differentiation factor 15 (GDF15), incretin (INC), insulin (INS), interleukin (IL), ciliary neurotrophic factor (CNTF), neuregulin (NRG) or relaxin (RLN).

48. The compound of claim 47, wherein the peptide or protein is an INC.

49. The compound of claim 48, wherein the INC is selected from the group consisting of glucose-dependent insulinotropic peptide (GIP), glucagon-like peptide-1 (GLP-1) and GIP/GLP-1.

50. The compound of claim 47, wherein the peptide or protein is IL, and wherein the IL is IL-2.

51. The compound of claim 47, wherein the peptide or protein is NRG, and wherein the NRG is neuregulin 1 (NRG1) or neuregulin 4 (NRG4).

52. The compound of claim 47, wherein the peptide or protein is RLN, and wherein the RLN is relaxin 2 (RLN2).

53. A pharmaceutical composition comprising a compound of claim 42 and a pharmaceutically acceptable buffer.

54. The pharmaceutical composition of claim 53 further comprising an additional therapeutic agent.

55. A method of treating an individual, the method comprising the step of:
administering to the individual an effective amount of a compound of claim 42.

56. A compound of claim 1 for use in extending the t½ of a biotherapeutic.

57-59. (canceled)

60. A compound comprising any one of SEQ ID NOS: 100, 101, 102, 103, 104, 105, 106, 107, 108, 109, 110, 111, 112, 113, 114, 115, 116, 117 or 118, or at least about 90% sequence similarity thereto.

61. A pharmaceutical composition comprising a compound of claim 60 and a pharmaceutically acceptable buffer.

62. The pharmaceutical composition of claim 61 further comprising an additional therapeutic agent.

63. A method of treating an individual, the method comprising the step of:
administering to the individual an effective amount of a compound of claim 60.

64-66. (canceled)
